(19) United States
(12) Patent Application Publication
(10) Pub. No.: US 2024/0131160 A1
(43) Pub. Date: Apr. 25, 2024

(54) TARGETING T REGULATORY CELLS TO ISLET CELLS TO STALL OR REVERSE TYPE 1 DIABETES

(71) Applicant: THE TRUSTEES OF THE UNIVERSITY OF PENNSYLVANIA, Philadelphia, PA (US)

(72) Inventors: James L. Riley, Downingtown, PA (US); Gavin Ellis, Philadelphia, PA (US)

(21) Appl. No.: 18/548,481

(22) PCT Filed: Mar. 1, 2022
(86) PCT No.: PCT/US2022/018248

§ 371 Date: Aug. 29, 2023

Related U.S. Application Data
(60) Provisional application No. 63/200,343, filed on Mar. 2, 2021.

(51) Int. Cl. A61K 39/00; C07K 14/705; C07K 14/725; C07K 16/40

(52) U.S. Cl. CPC A61K 39/46433; A61K 39/4611; A61K 39/4631; C07K 14/7051; C07K 14/70517; C07K 14/70521; C07K 16/40; C07K 2317/22; C07K 2317/569; C07K 2319/02; C07K 2319/03; C07K 2319/30; C07K 2319/33

(57) ABSTRACT

The present invention includes compositions and methods for an DPP6 specific chimeric antigen receptor (CAR). In certain embodiments the DPP6 specific CAR is expressed on a T regulatory cell. In certain embodiments, the DPP6 specific CAR is used to treat type 1 diabetes.

### CROSS-REFERENCE TO RELATED APPLICATION

The current application claims priority under 35 U.S.C. § 119(e) to U.S. Provisional Patent Application No. 63/200,343, filed Mar. 2, 2021, which is hereby incorporated by reference in its entirety herein.

### BACKGROUND OF THE INVENTION

Type 1 diabetes, also known as juvenile diabetes, insulin-dependent diabetes, and type 1 diabetes mellitus (T1DM) is characterized by insufficient production of insulin by the pancreas and resulting hyperglycemia. The primary cause of type 1 diabetes is an autoimmune reaction directed against β-islet cells in the pancreas which produce insulin. Beginning with an autoantibody reaction often early in life, type 1 diabetes progresses to a T cell-mediated condition that gradually decreases the number of β-islet cells, or β-cell mass in a patient until all cells are essentially lost. If left untreated, the type-1 diabetes related hyperglycemia leads to diabetic ketoacidosis, coma, and death. Treatment of type 1 diabetes has typically been managed through diet restrictions and the active monitoring of blood glucose levels and administration of exogenous insulin via injection. While these clinical strategies greatly extend normal functioning, they do not treat the underlying cause of the disease and require constant, lifelong, and painful blood sampling and injections, both of which involve costly materials constant attention.

Thus a need exists for treatments of type 1 diabetes that would reduce or reverse the autoimmune responses that cause the disease in a way that leads to long-term reduction or reversal of β-cell loss after single or few treatments. The current invention addresses this need.

### SUMMARY OF THE DISCLOSURE

The present invention includes compositions and methods for treatment of type 1 diabetes (T1D) comprising modified regulatory T cells comprising a chimeric antigen receptor (CAR) construct specific for dipeptidyl peptidase like protein 6 (DPP6).

As such, in one aspect the invention includes a modified immune cell or precursor cell thereof, comprising a chimeric antigen receptor (CAR) having affinity for DPP6, wherein the CAR comprises a DPP6 binding domain, a transmembrane domain, and an intracellular domain.

In certain embodiments, the DPP6 binding domain is a nanobody.

In certain embodiments, the DPP6 binding domain comprises a heavy chain variable region comprising a first CDR region comprising an amino acid sequence selected from the group set forth in SEQ ID NOs: 3, 19, 28, and 35; a second CDR region comprising an amino acid sequence selected from the group set forth in SEQ ID NOs: 4, 20, 29, and 36; and a third CDR region comprising an amino acid sequence selected from the group consisting of AT and set forth in SEQ ID NOs: 21, 30, and 37.

In certain preferred embodiments, the DPP6 binding domain comprises a heavy chain variable region comprising the amino acid sequence set forth in SEQ ID NOs: 1, 17, 26, and 33.

In certain embodiments, the CAR further comprises a hinge domain.

In certain embodiments, the hinge domain comprises a CD8 hinge.

In certain preferred embodiments, the CD8 hinge comprises the amino acid sequence set forth in SEQ ID NO: 22. In certain embodiments, the CAR further comprises a spacer domain.

In certain embodiments, the spacer domain is a human IgG4 spacer domain.

In certain preferred embodiments, the human IgG4 spacer domain comprises the amino acid sequence set forth in SEQ ID NO: 7.

In certain embodiments, the transmembrane domain comprises a CD28 transmembrane domain.

In certain preferred embodiments, the transmembrane domain comprises the amino acid sequence set forth in SEQ ID NO: 10.

In certain embodiments, the intracellular domain comprises a CD28 costimulatory domain. In certain preferred embodiments, the CD28 costimulatory domain comprises the amino acid sequence set forth in SEQ ID NO: 11.

In certain embodiments, the intracellular domain comprises a CD3 domain.

In certain preferred embodiments, the CD3 domain comprises the amino acid sequence set forth in SEQ ID NO: 13. In certain embodiments, the intracellular domain comprises a CD28 costimulatory domain and a CD3 domain.

In certain embodiments, the CAR further comprises a CD8 signal peptide.

In certain preferred embodiments, the signal peptide comprises the amino acid sequence set forth in SEQ ID NO: 5.

In another aspect, the invention includes a modified immune cell or precursor cell thereof, comprising a chimeric antigen receptor (CAR) having affinity for DPP6, wherein the CAR comprises an DPP6 binding domain, a CD8 hinge domain, a CD28 transmembrane domain, a CD28 costimulatory domain, and a CD3 intracellular domain.

In another aspect, the invention provides a modified immune cell or precursor cell thereof, comprising a chimeric antigen receptor (CAR) having affinity for DPP6, wherein the CAR comprises an DPP6 binding domain, a human IgG4 hinge domain, a CD28 transmembrane domain, a CD28 costimulatory domain, and a CD3 intracellular domain.

In certain preferred embodiments, the CAR comprises the amino acid sequence set forth in SEQ ID NOs: 15, 24, 31, and 38.

In certain embodiments of the above aspects or any aspect or embodiment disclosed herein, the modified cell is a regulatory T cell.

In certain embodiments of the above aspects or any aspect or embodiment disclosed herein, the modified cell is an autologous cell.

In certain embodiments of the above aspects or any aspect or embodiment disclosed herein, the modified cell is derived from a human.

In another aspect, the invention includes an isolated nucleic acid, comprising a nucleic acid sequence encoding a chimeric antigen receptor (CAR) having affinity for DPP6, wherein the CAR comprises a DPP6 binding domain, a transmembrane domain, and an intracellular domain.

In certain embodiments, the DPP6 binding domain comprises a nanobody.

In certain embodiments, the DPP6 binding domain comprises a heavy chain variable region comprising a first CDR region comprising an amino acid sequence selected from the group set forth in SEQ ID NOs: 3, 19, 28, and 35; a second CDR region comprising an amino acid sequence selected from the group set forth in SEQ ID NOs: 4, 20, 29, and 36; and a third CDR region comprising an amino acid sequence selected from the group set forth in SEQ ID NOs: 5, 21, 30, and 37.

In certain preferred embodiments, the DPP6 binding domain comprises a heavy chain variable region comprising a nucleic acid sequence set forth in SEQ ID NOs: 2, 18, 27, and 34.

In certain embodiments, the CAR comprises a CD28 transmembrane domain.

In certain preferred embodiments, the CD28 transmembrane domain comprises a nucleic acid sequence set forth in SEQ ID NO: 9.

In certain embodiments, the intracellular domain comprises a CD28 costimulatory domain.

In certain preferred embodiments, the CD28 costimulatory domain comprises a nucleic acid sequence set forth in SEQ ID NO: 12.

In certain embodiments, the intracellular domain comprises a CD3 domain.

In certain preferred embodiments, the CD3 domain comprises a nucleic acid sequence set forth in SEQ ID NO: 14.

In certain preferred embodiments, the nucleic acid comprises a nucleic acid sequence selected from the group set forth in SEQ ID NOs: 16, 25, 32, and 39.

In another aspect, the invention includes an expression construct comprising the isolated nucleic acid of any of the above aspects or any aspect or embodiment disclosed herein.

In another aspect, the invention includes a method for generating the modified immune cell or precursor cell thereof of the above aspects or any aspect or embodiment disclosed herein, comprising introducing into the immune cell the nucleic acid of any one the above aspects or any aspect or embodiment disclosed herein, or the expression construct of the above aspects or any aspect or embodiment disclosed herein. In another aspect, the invention includes a method of treating an autoimmune disease in a subject in need thereof, comprising administering to the subject an effective amount of the modified immune cell or precursor cell thereof of any one of the above aspects or any aspect or embodiment disclosed herein.

In certain embodiments, the autoimmune disease is type 1 diabetes.

In another aspect, the invention includes a method of treating type 1 diabetes in a subject in need thereof, comprising administering to the subject a modified regulatory T cell comprising a chimeric antigen receptor (CAR) having affinity for DPP6, wherein the CAR comprises an DPP6 binding domain, a CD8 hinge domain, a CD28 transmembrane domain, a CD28 costimulatory domain, and a CD3 intracellular domain.

In another aspect, the invention includes a method of treating type 1 diabetes in a subject in need thereof, comprising administering to the subject a modified regulatory T cell comprising a chimeric antigen receptor (CAR) having affinity for DPP6, wherein the CAR comprises an DPP6 binding domain, a human IgG4 hinge domain, a CD28 transmembrane domain, a CD28 costimulatory domain, and a CD3 intracellular domain.

In certain embodiments, the modified cell is a modified regulatory T cell.

In certain embodiments, the modified cell is an autologous cell.

In certain embodiments of the above aspects or any aspect or embodiment disclosed herein the modified cell is derived from a human.

In another aspect, the invention includes a non-human primate model of type 1 diabetes, comprising administering to a non-human primate subject an effective amount of a modified T cell comprising a chimeric antigen receptor (CAR) having an affinity for a islet cell antigen.

In certain embodiments, the CAR has an affinity for DPP6.

In certain embodiments, the CAR has an affinity for fibroblast activation protein (FAP).

In another aspect, the invention includes a non-human primate model of type 1 diabetes, comprising administering to a non-human primate subject an effective amount of a first modified T cell comprising a CAR having an affinity for a islet cell antigen and a second modified T cell comprising a CAR having an affinity for a different islet cell antigen.

In certain embodiments, the first islet cell antigen is DPP6 and the second islet cell antigen is FAP.

In certain embodiments, the modified T cells are administered intravenously.

In certain embodiments, the modified T cells are administered via the splenic artery.

In certain embodiments, the non-human primate model further comprises the administration of streptozotocin, wherein the amount of streptozotocin is sufficient to induce islet cell injury but not depletion.

In certain embodiments, the non-human primate model further comprises the administration of an effective amount of an immune-modulating agent.

In certain embodiments, the immune-modulating agent is a CRISPR-based system.

In certain embodiments, the CRISPR-based system disrupts the expression of an immune checkpoint protein.

In certain embodiments, the immune checkpoint protein is selected from the group consisting of PD-1, CTLA-4, TIM3, GITR, BTLA, LAG3, and any combination thereof.

In certain embodiments, the subject is selected from the group consisting of a rhesus macaque, a cynomolgus macaque, a chimpanzee, and a baboon.

In another aspect, the invention includes a non-human primate animal model of diabetes made by the method of any of the above aspects or any aspect or embodiment disclosed herein.

In another aspect, the invention includes a method of treating type 1 diabetes in a subject in need thereof, comprising administering to the subject a modified regulatory T cell comprising a chimeric antigen receptor (CAR) having affinity for FAP, wherein the CAR comprises an FAP binding domain, a CD28 transmembrane domain, a CD28 costimulatory domain, and a CD3ζ intracellular domain.

In certain embodiments, the modified cell is an autologous cell.

In certain embodiment, the modified cell is derived from a human.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1 shows a diagram of the anti-DPP6, VHH-based CARs of the invention.

FIG. 2 is a table illustrating the binding affinities of the various anti-DPP6 nanobodies used in the creating of the CARs of the invention. Data in this table are taken from Balhuizen et al. (2017) Scientific Reports 7: 15130.

FIG. 3 describes the workflow of an in vitro study demonstrating the antigen-recognition ability of anti-DPP6 CARs.

FIG. 4 is a series of dot plots demonstrating the expression and transduction efficiency of two anti-DPP6 CARs in normal human donor T cells as measured by flow cytometry using an anti-camel primary antibody.

FIG. 5 is a series of dot plots demonstrating the expression and transduction efficiency of a mouse-derived anti-CD19 CAR construct in normal human donor T cells which is used as a control in the present studies.

FIG. 6 is a series of dot plots demonstrating the expression and transduction efficiency of the 4hD29-28z CAR using an antibody specific for the human IgG spacer.

FIG. 7 shows that the anti-DPP6 CARs are able to activate CD4 T cells expressing them when exposed to DPP6-expressing Kelly neuroblastoma cells. PMA/Ionomycin is used as a positive control for activation. Cells expressing the anti-CD19-28z cells were used as a control.

FIG. 8 shows that the anti-DPP6 CARs are able to activate CD8 T cells expressing them when exposed to DPP6-expressing Kelly neuroblastoma cells. PMA/Ionomycin is used as a positive control for activation. Cells expressing the anti-CD19-28z cells were used as a control.

FIG. 9 shows a diagram of the setup of an in vitro cytotoxicity assay.

FIG. 10 is a graph showing that T cells expressing each of the four anti-DPP6 CARs are able to suppress the growth of DPP6-expressing target cells at an effector to target ratio of 10:1.

FIG. 11 is a graph showing that T cells expressing each of the four anti-DPP6 CARs are able to suppress the growth of DPP6-expressing target cells at an effector to target ratio of 1:1.

FIG. 12 is a graph showing that T cells expressing each of the four anti-DPP6 CARs are able to suppress the growth of DPP6-expressing target cells at an effector to target ratio of 1:10.

FIG. 13 is a diagram of the experimental setup for an in vivo assay to demonstrate the function of anti-DPP6 CAR expressing T cells. In these experiments, effector T cells are expressing the anti-DPP6 CARs so β-cell toxicity and hyperglycemia were readouts of CAR function.

FIG. 14 is a pair of micrographs showing that 4hD29-28z CAR expressing T cells are able to infiltrate the kidney capsule into regions containing β-cells.

FIG. 15 is a micrograph from the same mouse as FIG. 14 showing destruction of insulin-producing β-cells.

FIG. 16 is a pair of micrographs from another mouse receiving 4hD29-28z CAR expressing T cells showing infiltration of T cells into the kidney capsule.

FIG. 17. is a micrograph from the mouse in FIG. 16 showing depletion of insulin-producing cells in regions containing T cell infiltration.

FIG. 18 is a pair of micrographs from a mouse receiving 2hD6-28z CAR expressing T cells showing T cell infiltration into the kidney capsule.

FIG. 19 is a micrograph from the same mouse as FIG. 18 showing depletion of insulin-producing cells in regions of the kidney capsule containing infiltrations of T cells.

FIG. 20 is a micrograph from a control mouse which did not receive any CAR expressing T cells.

FIG. 21 is another micrograph from the same mouse as FIG. 20 showing an absence of Treg and CD8+ T cell markers in the kidney capsule tissue.

FIG. 22 is a micrograph from the same mouse as FIG. 20 showing an example of normal distribution of insulin-producing cells in these animals.

FIG. 23 illustrates the expression of the 2hD38-28z CAR on expanded human CD4+ Tregs. Tregs were sorted, stimulated and transduced with 2hD38-28z virus. On day 9, the cells were restimulated by plating in flask containing irradiated Kelly cells. On day 13, they were stained for human CAR (left) or VHH (right).

FIG. 24 illustrates the activation of expanded 2hD38-28z CAR expressing CD4+ Treg cells. Treg cells were transduced and expanded for 16 days before overnight activation with αCD3/αCD28 microbeads or DPP6-expressing Kelly neuroblastoma cells. Two K562 cell lines were used as negative controls. GARP expression was used as a marker of activation.

FIG. 25 illustrates an in vitro suppression assay using expanded 2hD38-28z CAR expressing CD4+ Tregs in either uncoated wells (K562.A2.SL9.19) or wells coated with irradiated Kelly cells (K562.A2.SL9.19+Kelly). After 4 days, CFSE dilution of bystander CD8 T cells was measured by flow cytometry and percent suppression was calculated.

FIG. 26 shows anti-DPP6 CAR expressing T cells demonstrating reactivity against stem-cell derived beta cells. Three of the DPP6 CARs T cells tested reacted with the stem cell derived beta cells and secreted cytokines. HLA-A2 CAR T cells reactivity was expected, as the beta cells were derived from an HLA-A2+ donor.

FIG. 27 shows two CARs bearing antigen-binding scFv domains specific for SCA-B1 and SCA-B5 β-islet cell markers being stimulated with either human islet cells or non-specifically with PMA/ionomycin. CAR-expressing T cells show no recognition of (3-islet cells.

FIG. 28 illustrates that DPP6- and FAP-specific CARs recognize non-human primate (NHP) islet cells. Fresh islets were left intact (whole) or mechanically dissociated and mixed with the indicated CART cells for 4 hours. Intracellular MIP-1β was measured by flow cytometry. HLA-A2 CAR T cells were used as a negative control.

FIG. 29 illustrates the progression of blood glucose levels in STZ-treated mice transplanted with NHP islet cells. Ten STZ treated mice were confirmed to be diabetic and then transplanted with NHP islets. After 7 days of normoglycemia, 2 mice were left alone; 4 mice received control CAR T cells (A2) and 5 mice received DPP6-specific CAR T cells.

FIG. 30 is a pair of micrographs showing that FAP CAR expressing T cells are able to infiltrate the kidney capsule into regions containing β-cells.

FIG. 31 is a micrograph from the same mouse as FIG. 30 showing an example of normal distribution of insulin-producing cells in these animals.

FIG. 32 illustrates human Tregs transduced with FAP-28z CAR or irrelevant CAR virus and stained for CAR construct expression via flow cytometry.

FIG. 33 illustrates an in vitro suppression assay in which human Tregs transduced to express the FAP-28z CAR or and irrelevant CAR were co-cultured with CellTrace Violet responder cells and aAPCs expressing FAP.

FIG. 34 is a graph illustrating the calculated percent suppression for the in vitro suppression assay in FIG. 33. The numbers are the percent suppression calculated by: (1−(#of cell divisions in that well/#of cell divisions per cell in no tregs well))*100. Similar to the experiment illustrated in FIG. 25.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. “About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division. As used herein, to “alleviate” a disease means reducing the severity of one or more symptoms of the disease.

“Allogeneic” refers to a graft derived from a different animal of the same species. “Alloantigen” refers to an antigen present only in some individuals of a species and capable of inducing the production of an alloantibody by individuals which lack it. The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. α and β light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual. “Allogeneic” refers to any material derived from a different animal of the same species.

The tem “chimeric antigen receptor” or “CAR.” as used herein, refers to an artificial cell receptor that is engineered to be expressed on an immune effector cell and specifically bind an antigen. CARs may be used as a therapy with adoptive cell transfer. T cells are removed from a patient and modified so that they express the receptors specific to a particular form of antigen. In some embodiments, the CAR has specificity to a selected target, for example a human leukocyte antigen MLA). CARs may also comprise an intracellular activation domain, a transmembrane domain and an extracellular domain comprising an antigen binding region, in some aspects, CARs comprise an extracellular domain comprising an anti-HLA binding domain fused to CD8 hinge domain, a CD28 transmembrane and intracellular domain, and a CD3-zeta domain.

The term “cleavage” refers to the breakage of covalent bonds, such as in the backbone of a nucleic acid molecule or the hydrolysis of peptide bonds. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides may be used for targeting cleaved double-stranded DNA.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

“Co-stimulatory ligand,” as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-IBB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Donor antigen” refers to an antigen expressed by the donor tissue to be transplanted into the recipient.

“Recipient antigen” refers to a target for the immune response to the donor antigen.

The term “downregulation” as used herein refers to the decrease or elimination of gene expression of one or more genes. “Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“HLA-A2” refers to a human leukocyte antigen within the HLA-A serotype group. HLA-A is one of the three major types of MHC class I cell surface receptors. The other two types are HLA-B and HLA-C. The HLA complex helps the immune system distinguish between the body's own proteins and foreign proteins, e.g., those that come from an organ transplantation. HLA is the human version of the major histocompatibility complex (MHC), a gene family that is present in many species. MHC genes are separated into three groups: class I, class II, and class III. MHC class I molecules are one of two (the other being MHC class II) primary classes of major histocompatibility complex (MHC) molecules that are found on the cell surface of cells. The function of MHC class I molecules is to display peptide fragments of non-self proteins from within the cell to immune cells (e.g., cytotoxic T cells), resulting in the trigger of an immediate response from the immune system against the particular non-self-antigen that is displayed.

“HLA-A28” refers to a human leukocyte antigen within the HLA-A serotype group. “HLA-A68” refers to a human leukocyte antigen within the HLA-A serotype group. The alpha “A” chain is encoded by the HLA-A*68 allele group and the β-chain is encoded by the 13-2 microglobulin (B2M) locus.

“DPP6” refers to a human leukocyte antigen within the HLA-B serotype group.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody, where the whole molecule is of human origin or consists of an amino acid sequence identical to a human form of the antibody.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “immune response” as used herein is defined as a cellular response to an antigen that occurs when lymphocytes identify antigenic molecules as foreign and induce the formation of antibodies and/or activate lymphocytes to remove the antigen.

The term “immunostimulatory” is used herein to refer to increasing overall immune response.

The term “immunosuppressive” is used herein to refer to reducing overall immune response.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockout” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The term “limited toxicity” as used herein, refers to the peptides, polynucleotides, cells and/or antibodies of the invention manifesting a lack of substantially negative biological effects, anti-tumor effects, or substantially negative physiological symptoms toward a healthy cell, non-tumor cell, non-diseased cell, non-target cell or population of such cells either in vitro or in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “self-antigen” as used herein is defined as an antigen that is expressed by a host cell or tissue. Self-antigens may be tumor antigens, but in certain embodiments, are expressed in both normal and tumor cells. A skilled artisan would readily understand that a self-antigen may be overexpressed in a cell.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals.

Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (a) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic I cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

“Transplant” refers to a biocompatible lattice or a donor tissue, organ or cell, to be transplanted. An example of a transplant may include but is not limited to skin cells or tissue, bone marrow, and solid organs such as heart, pancreas, kidney, lung and liver. A transplant can also refer to any material that is to be administered to a host. For example, a transplant can refer to a nucleic acid or a protein.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject. A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like. “Xenogeneic” refers to any material derived from an animal of a different species.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Peripheral blood contains a small population of T cell lymphocytes that express the T regulatory phenotype (“Treg”), i.e., are positive for both CD4 and CD25 antigens. There are several subsets of Treg cells. One subset of regulatory cells develops in the thymus. Thymic derived Treg cells function by a cytokine-independent mechanism, which involves cell to cell contact. They are essential for the induction and maintenance of self-tolerance and for the prevention of autoimmunity. These regulatory cells prevent the activation and proliferation of autoreactive T cells that have escaped thymic deletion or recognize extrathymic antigens, thus they are critical for homeostasis and immune regulation, as well as for protecting the host against the development of autoimmunity. Thus, immune regulatory CD4⁺CD25⁺ T cells are often referred to as “professional suppressor cells.”

DPP6 or dipeptidyl aminopeptidase-like protein 6 (also known as DPPX) is a single-pass type II membrane glycoprotein that is a member of the S9B family of serine proteases, however DPP6 lacks any detectable protease activity due to mutations in the catalytic domain. DPP6 protein forms multimeric complexes with the KV4.2 potassium ion channel proteins, and regulates the voltage-dependent gating properties and expression of KV4.2 in the central nervous system. Recent RNA sequencing studies have identified DPP6 as being highly expressed in human pancreatic islets as compared to surrounding tissue, and this overexpression is not affected by proinflammatory cytokine signaling. In this way, DPP6 has been identified as biomarker of islet cells.

FAP or fibroblast activation protein is a cell-surface serine protease which modulates the bioavailability and, by extension, the function of a number of peptides with biological activity including hormones and extracellular matrix molecules. FAP has been found to be highly expressed by alpha cells in human pancreatic islets.

The present invention includes compositions and methods for utilizing DPP6- and FAP-specific CAR constructs to protect pancreatic islet cells from T cell responses associated with autoimmune diseases including type I diabetes. The present invention is based on the finding that regulatory T cells comprising an DPP6 or FAP specific CAR are capable of suppressing autoimmune responses in an antigen-specific manner.

The DPP6 specific CAR comprises an antigen binding domain that binds to DPP6. When expressed on human CD4+ T regulatory cells (Tregs), the DPP6 specific CAR mediates antigen specific suppression. The DPP6 specific CAR is able to redirect T regulatory cells to DPP6 expressing tissue and suppress inflammatory T cell responses, including autoimmune T cell responses.

The FAP specific CAR comprises and antigen binding domain that binds to FAP. When expressed on human CD4+ T regulatory cells (Tregs), the FAP specific CAR mediates antigen specific suppression. The FAP specific CAR is able to redirect T regulatory cells to FAP expressing tissue and suppress inflammatory T cell responses, including autoimmune T cell responses.

In certain embodiments, the DPP6 CAR comprises a DPP6 epitope-binding nanobody with intracellular T cell activation and costimulatory domains. Transduced T cells expressing the DPP6 CAR become activated in response to binding the DPP6 antigen found on α- and β-islet cells from humans and other mammals.

In certain embodiments, the FAP CAR comprises a FAP epitope-binding scFv with intracellular T cell activation and costimulatory domains. Transduced T cells expressing the FAP CAR become activated in response to binding the FAP antigen found on α-islet cells from humans and other mammals.

One application of this method is as part of regulatory T cell adoptive therapy designed to treat type I diabetes. Sorted regulatory T cells from an individual are grown and induced to express the transgenic DPP6 or FAP CAR molecule in vitro. The cells are transferred to a subject suffering from type I diabetes. The recipient may or may not be the original source of the Tregs. DPP6 or FAP CAR Tregs may improve upon current type I diabetes treatment regimens by obviating the need to maintain daily blood monitoring and insulin injections.

### Chimeric Antigen Receptor (CAR)

The present invention provides compositions and methods for modified immune cells or precursor cells thereof, e.g., modified regulatory T cells, comprising a chimeric antigen receptor (CAR) having affinity for DPP6 or FAP. A subject CAR of the invention comprises an antigen binding domain (e.g., DPP6 or FAP binding domain), a transmembrane domain, and an intracellular domain. A subject CAR of the invention may comprise a hinge domain or a spacer domain, and/or a signal peptide. As known in the art, when the subject CAR is translated, it contains the signal peptide to direct the molecule to the cell surface. This signal peptide is then cleaved off. While the signal peptide is not part of the antigen-recognizing CAR at the cell surface of the modified immune cell (e.g., DPP6 or FAP specific CAR), it is useful for the CAR's function. In some embodiments, the signal peptide is a CD8 signal peptide. Accordingly, a subject CAR of the invention comprises an antigen binding domain (e.g., DPP6 or FAP binding domain), a hinge domain or spacer domain, a transmembrane domain, and an intracellular domain. In some embodiments, a subject CAR of the invention comprises a signal peptide, an antigen binding domain (e.g., DPP6 or FAP binding domain), a hinge domain or spacer domain, a transmembrane domain, and an intracellular domain. In some embodiments, each of the domains of a subject CAR is separated by a linker.

The antigen binding domain may be operably linked to another domain of the CAR, such as the transmembrane domain or the intracellular domain, both described elsewhere herein, for expression in the cell. In one embodiment, a first nucleic acid sequence encoding the antigen binding domain is operably linked to a second nucleic acid encoding a transmembrane domain, and further operably linked to a third a nucleic acid sequence encoding an intracellular domain.

The antigen binding domains described herein can be combined with any of the transmembrane domains described herein, any of the intracellular domains or cytoplasmic domains described herein, or any of the other domains described herein that may be included in a CAR of the present invention.

In one aspect, the invention includes an isolated DPP6 specific chimeric antigen receptor (CAR) comprising a CD8 signal peptide, an DPP6 VHH domain, a CD8 hinge region or a human IgG4 spacer region, a CD28 transmembrane domain, a CD28 costimulatory domain, and a CD3-zeta intracellular domain. In another aspect, the invention includes an isolated nucleic acid encoding an DPP6 specific CAR, wherein the CAR comprises an DPP6 VHH domain, a CD8 hinge region or a human IgG4 spacer region, a CD28 transmembrane domain, a CD28 costimulatory domain, and a CD3-zeta intracellular domain. Another aspect of the invention includes an isolated polypeptide comprising an DPP6 VHH domain, a CD8 hinge region or a human IgG4 spacer region, a CD28 transmembrane domain, a CD28 costimulatory domain, and a CD3-zeta intracellular domain.

Another aspect of the invention includes a genetically modified T cell (e.g., regulatory T cell) comprising an isolated nucleic acid encoding an DPP6 specific CAR, wherein the CAR comprises an DPP6 VHH domain, a CD8 hinge region or a human IgG4 spacer region, a CD28 transmembrane domain, a CD28 costimulatory domain, and a CD3-zeta intracellular domain. In some embodiments, a genetically modified immune cell (e.g., T cell, regulatory T cell) of the present invention comprises an DPP6 CAR, wherein the CAR comprises an DPP6 VHH domain, a CD8 hinge region or a human IgG4 spacer region, a CD28 transmembrane domain, a CD28 costimulatory domain, and a CD3-zeta intracellular domain. In some embodiments, a genetically modified immune cell (e.g., T cell, regulatory T cell) or precursor cell thereof of the present invention comprises a chimeric antigen receptor (CAR) having affinity for DPP6. The CAR comprises an DPP6 binding domain, a CD8 hinge domain or a human IgG4 spacer domain, a CD8 signal peptide, a CD28 transmembrane domain, a CD28 costimulatory domain, and a CD3ζ intracellular domain.

Another aspect of the invention includes a genetically modified T cell (e.g., regulatory T cell) comprising an isolated nucleic acid encoding a FAP specific CAR, wherein the CAR comprises an FAP-binding domain, a CD8 hinge region or a human IgG4 spacer region, a CD8 transmembrane domain, a CD28 costimulatory domain, and a CD3-zeta intracellular domain. In some embodiments, a genetically modified immune cell (e.g., T cell, regulatory T cell) of the present invention comprises a FAP CAR, wherein the CAR comprises a FAP binding domain, a CD28 transmembrane domain, a CD28 costimulatory domain, and a CD3-zeta intracellular domain. In some embodiments, a genetically modified immune cell (e.g., T cell, regulatory T cell) or precursor cell thereof of the present invention comprises a chimeric antigen receptor (CAR) having affinity for FAP. The CAR comprises a FAP binding domain, a CD8 signal peptide, a CD28 transmembrane domain, a CD28 costimulatory domain, and a CD3ζ intracellular domain. In certain embodiments of the invention, the CAR is encoded by the nucleic acid sequences of SEQ ID NOs: 16, 25, 32,39, and 63. In other embodiments, the CAR comprises the amino acid sequences of SEQ ID NOs: 15, 24, 31, and 38.

In certain embodiments, the genetically modified T cell is a T regulatory (Treg) cell.

Sequences of individual domains and a subject CARs of the present invention are found in Table 1.

TABLE 1

| SEQ | | |
|---|---|---|
| ID | | |
| NO: | Description | Sequence |
| 1. | 4hD29 nanobody | QVQLQESGGGLVQPGGSLRLSCAASGFTFSSNYMTW |
| | amino acid | VRQAPGKGPEWVSGINPDGSSTYYADSVKGRFTISR |
| | sequence | DNAKNTLYLQMNSLKSEDTALYKCATGAAPRIPTTL |
| | | RGQGTQVTVSS |
| 2. | 4hD29 nanobody | caagtgcaacttcaagagtccggcggtggacttgtccagccaggagggagcctgc |
| | nucleic acid | gactttcctgcgccgccagtgggttcacattttccagtaattatatgacttgggtacga |
| | sequence | caggcacctggcaaaggccctgagtgggtctcaggaattaacccggatggttcca |
| | | gcacatactacgcggattctgttaagggccgctttaccatctcaagggataacgcta |
| | | agaataccctttatctccagatgaacagtctcaaaagcgaggacaccgcattgtata |
| | | aatgtgcaacaggggctgctcctaggattccaaccacattgagaggtcaaggcact |
| | | caggtgactgtctcctca |
| 3. | 4hD29 CDR1 | GFTFSSNY |
| | amino acid | |
| | sequence | |
| 4. | 4hD29 CDR2 | INPDGSST |
| | amino acid | |
| | sequence | |
| | 4hD29 CDR3 | AT |
| | amino acid | |
| | sequence | |
| 5. | CD8 signal | MALPVTALLLPLALLLHAARP |
| | peptide amino | |
| | acid sequence | |
| 6. | CD8 signal | atggccttaccagtgaccgccttgctcctgccgctggccttgctgctccacgccgcc |
| | peptide nucleic | aggccg |
| | acid sequence | |
| 7. | IgG4 hinge | ESKYGPPCPSCPAPEFEGGPSVFLFPPKPKDTLMISRT |
| | amino acid | PEVTCVVVDVSQEDPEVQFNWYVDGVEVHNAKTKP |
| | sequence | REEQFQSTYRVVSVLTVLHQDWLNGKEYKCKVSNK |
| | | GLPSSIEKTISKAKGQPREPQVYTLPPSQEEMTKNQVS |
| | | LTCLVKGFYPSDIAVEWESNGQPENNYKTTPPVLDS |
| | | DGSFFLYSRLTVDKSRWQEGNVFSCSVMHEALHNH |
| | | YTQKSLSLSLGK |
| 8. | IgG4 hinge | gagagcaagtacggccccccttgccccagctgtccggcaccagagttcgaaggg |
| | nucleic acid | ggaccctccgtctttctcttcccccctaagcccaaggataccctgatgatttcacgca |
| | sequence | ctcctgaggtaacttgcgtggtcgtagatgttagccaggaagatccagaagttcagtt |
| | | taattggtacgttgatggggtggaggtgcacaatgccaaaactaaacccagagagg |
| | | aacagttccagtctacttatcgagtggttagcgttctgaccgtcctgcaccaggactg |
| | | gctgaacggaaaggaatataaatgcaaggtcagtaacaaggggctccctagctcc |
| | | atcgagaaaacgatatcaaaagcaaagggacagccaagagagcctcaggtctac |
| | | actcttcctccaagccaggaagaaatgaccaaaaaccaggtttcccttacctgtctg |
| | | gttaagggcttttacccatccgatattgcggtggaatgggaatccaacggtcaaccg |
| | | gaaaacaattacaagaccacccctccggtcctggacagcgatggaagtttttttttgt |
| | | attccaggctcaccgtcgataaaagtaggtggcaggaaggaaacgtcttttcctgca |
| | | gtgtgatgcatgaagcgctgcataatcattatacacagaagtccttgagtcttagcctc |
| | | gggaaa |
| 9 | CD28 | ttttgggtgctggtggtggttggtggagtcctggcttgctatagcttgctagtaacagt |
| | transmembrane | ggcctttattattttctgggtg |
| | domain nucleic | |
| | acid sequence | |
| 10. | CD28 | FWVLVVVGGVLACYSLLVTVAFIIFWV |
| | transmembrane | |
| | domain amino | |
| | acid sequence | |
| 11. | CD28 | RSKRSRLLHSDYMNMTPRRPGPTRKHYQPYAPPRDF |
| | intracellular | AAYRS |
| | domain amino | |
| | acid sequence | |
| 12. | CD28 | aggagtaagaggagcaggctcctgcacagtgactacatgaacatgactccccgcc |
| | intracellular | gccccgggcccacccgcaagcattaccagccctatgccccaccacgcgacttcgc |
| | domain nucleic | agcctatcgctcc |
| | acid sequence | |
| 13. | CD3 zeta | RVKFSRSADAPAYQQGQNQLYNELNLGRREEYDVL |
| | domain amino | DKRRGRDPEMGGKPRRKNPQEGLYNELQKDKMAE |
| | acid sequence | AYSEIGMKGERRRGKGHDGLYQGLSTATKDTYDAL |
| | | HMQALPPR |
| 14. | CD3 zeta | agagtgaagttcagcaggagcgcagacgcccccgcgtaccagcagggccagaa |
| | domain nucleic | ccagctctataacgagctcaatctaggacgaagagaggagtacgatgttttggaca |
| | acid sequence | agagacgtggccgggaccctgagatggggggaaagccgagaaggaagaaccct |
| | | caggaaggcctgtacaatgaactgcagaaagataagatggcggaggcctacagtg |
| | | agattgggatgaaaggcgagcgccggaggggcaaggggcacgatggcctttacc |
| | | agggtctcagtacagccaccaaggacacctacgacgcccttcacatgcaggccct |
| | | gccccctcgc |
| 15. | 4hD29-28z CAR | MALPVTALLLPLALLLHAARPGSQVQLQESGGGLVQ |
| | amino acid | PGGSLRLSCAASGFTFSSNYMTWVRQAPGKGPEWVS |
| | sequence | GINPDGSSTYYADSVKGRFTISRDNAKNTLYLQMNS |
| | | LKSEDTALYKCATGAAPRIPTTLRGQGTQVTVSSESK |
| | | YGPPCPSCPAPEFEGGPSVFLFPPKPKDTLMISRTPEV |
| | | TCVVVDVSQEDPEVQFNWYVDGVEVHNAKTKPREE |
| | | QFQSTYRVVSVLTVLHQDWLNGKEYKCKVSNKGLP |
| | | SSIEKTISKAKGQPREPQVYTLPPSQEEMTKNQVSLTC |
| | | LVKGFYPSDIAVEWESNGQPENNYKTTPPVLDSDGSF |
| | | FLYSRLTVDKSRWQEGNVFSCSVMHEALHNHYTQK |
| | | SLSLSLGKFWVLVVVGGVLACYSLLVTVAFIIFWVRS |
| | | KRSRLLHSDYMNMTPRRPGPTRKHYQPYAPPRDFAA |
| | | YRSIDRVKFSRSADAPAYQQGQNQLYNELNLGRREE |
| | | YDVLDKRRGRDPEMGGKPRRKNPQEGLYNELQKDK |
| | | MAEAYSEIGMKGERRRGKGHDGLYQGLSTATKDTY |
| | | DALHMQALPPR |
| 16. | 4hD29-28z CAR | atggccttaccagtgaccgccttgctcctgccgctggccttgctgctccacgccgcc |
| | nucleic acid | aggccgggatcccaagtgcaacttcaagagtccggcggtggacttgtccagccag |
| | sequence | gagggagcctgcgactttcctgcgccgccagtgggttcacattttccagtaattatat |
| | | gacttgggtacgacaggcacctggcaaaggccctgagtgggtctcaggaattaac |
| | | ccggatggttccagcacatactacgcggattctgttaagggccgctttaccatctcaa |
| | | gggataacgctaagaataccctttatctccagatgaacagtctcaaaagcgaggac |
| | | accgcattgtataaatgtgcaacaggggctgctcctaggattccaaccacattgaga |
| | | ggtcaaggcactcaggtgactgtctcctcagagagcaagtacggccccccttgccc |
| | | cagctgtccggcaccagagttcgaagggggaccctccgtctttctcttcccccctaa |
| | | gcccaaggataccctgatgatttcacgcactcctgaggtaacttgcgtggtcgtagat |
| | | gttagccaggaagatccagaagttcagtttaattggtacgttgatggggtggaggtg |
| | | cacaatgccaaaactaaacccagagaggaacagttccagtctacttatcgagtggtt |
| | | agcgttctgaccgtcctgcaccaggactggctgaacggaaaggaatataaatgcaa |
| | | ggtcagtaacaaggggctccctagctccatcgagaaaacgatatcaaaagcaaag |
| | | ggacagccaagagagcctcaggtctacactcttcctccaagccaggaagaaatga |
| | | ccaaaaaccaggtttcccttacctgtctggttaagggcttttacccatccgatattgcg |
| | | gtggaatgggaatccaacggtcaaccggaaaacaattacaagaccacccctccgg |
| | | tcctggacagcgatggaagtttttttttgtattccaggctcaccgtcgataaaagtagg |
| | | tggcaggaaggaaacgtcttttcctgcagtgtgatgcatgaagcgctgcataatcatt |
| | | atacacagaagtccttgagtcttagcctcgggaaattttgggtgctggtggtggttgg |
| | | tggagtcctggcttgctatagcttgctagtaacagtggcctttattattttctgggtgag |
| | | gagtaagaggagcaggctcctgcacagtgactacatgaacatgactccccgccgc |
| | | cccgggcccacccgcaagcattaccagccctatgccccaccacgcgacttcgcag |
| | | cctatcgctccatcgatagagtgaagttcagcaggagcgcagacgcccccgcgta |
| | | ccagcagggccagaaccagctctataacgagctcaatctaggacgaagagagga |
| | | gtacgatgttttggacaagagacgtggccgggaccctgagatggggggaaagcc |
| | | gagaaggaagaaccctcaggaaggcctgtacaatgaactgcagaaagataagat |
| | | ggcggaggcctacagtgagattgggatgaaaggcgagcgccggaggggcaag |
| | | gggcacgatggcctttaccagggtctcagtacagccaccaaggacacctacgacg |
| | | cccttcacatgcaggccctgccccctcgctaa |
| 17. | 2hD1 nanobody | QVQLQESGGGLVQPGGSLRLSCAASGYPYGYTFSSY |
| | amino acid | CMRWFRQAPGKDREGVARFERNGLTTYYDDSVKGR |
| | sequence | FTISQDNVKNTVYLQMNSLKPEDTATYYCAAAPKQL |
| | | RTCGDYNYWGQGTQVTVSS |
| 18. | 2hD1 nanobody | caggttcaattgcaagagtccggcggcgggcttgtgcaaccaggcggcagcttgc |
| | nucleic acid | gcctctcatgtgctgcatccggttacccatatggctacacgtttagttcttactgtatgc |
| | sequence | gatggttccggcaggcgcctgggaaggatcgggaaggggttgctaggtttgaaag |
| | | aaacggacttaccacttactatgacgactccgttaaaggtagatttactatatcccagg |
| | | acaatgtgaaaaataccgtgtacttgcaaatgaatagcttgaaaccagaggacactg |
| | | caacatactattgcgctgctgccccaaaacaactgaggacgtgtggggattacaact |
| | | attgggggcaaggcactcaagtcactgtcagttct |
| 19. | 2hD1 CDR1 | GYTFSSYC |
| | amino acid | |
| | sequence | |
| 20. | 2hD1 CDR2 | FERNGLTT |
| | amino acid | |
| | sequence | |
| 21. | 2hD1 CDR3 | AAAPKQLRTCGDYNY |
| | amino acid | |
| | sequence | |
| 22. | CD8 hinge | TTTPAPRPPTPAPTIASQPLSLRPEACRPAAGGAVHTR |
| | amino acid | GLDFACD |
| | sequence | |
| 23. | CD8 hinge | accacgacgccagcgccgcgaccaccaacaccggcgcccaccatcgcgtcgca |
| | nucleotide | gcccctgtccctgcgcccagaggcgtgccggccagcggggggggcgcagtgc |
| | sequence | acacgagggggctggacttcgcctgtgat |
| 24. | 2hD1-28z CAR | MALPVTALLLPLALLLHAARPGSQVQLQESGGGLVQ |
| | amino acid | PGGSLRLSCAASGYPYGYTFSSYCMRWFRQAPGKDR |
| | sequence | EGVARFERNGLTTYYDDSVKGRFTISQDNVKNTVYL |
| | | QMNSLKPEDTATYYCAAAPKQLRTCGDYNYWGQG |
| | | TQVTVSSSGTTTPAPRPPTPAPTIASQPLSLRPEACRPA |
| | | AGGAVHTRGLDFACDFWVLVVVGGVLACYSLLVTV |
| | | AFIIFWVRSKRSRLLHSDYMNMTPRRPGPTRKHYQP |
| | | YAPPRDFAAYRSIDRVKFSRSADAPAYQQGQNQLYN |
| | | ELNLGRREEYDVLDKRRGRDPEMGGKPRRKNPQEG |
| | | LYNELQKDKMAEAYSEIGMKGERRRGKGHDGLYQG |
| | | LSTATKDTYDALHMQALPPR |
| 25. | 2hD1-28z CAR | atggccttaccagtgaccgccttgctcctgccgctggccttgctgctccacgccgcc |
| | nucleic acid | aggccgggatcccaggttcaattgcaagagtccggcggcgggcttgtgcaaccag |
| | sequence | gcggcagcttgcgcctctcatgtgctgcatccggttacccatatggctacacgtttag |
| | | ttcttactgtatgcgatggttccggcaggcgcctgggaaggatcgggaaggggttg |
| | | ctaggtttgaaagaaacggacttaccacttactatgacgactccgttaaaggtagattt |
| | | actatatcccaggacaatgtgaaaaataccgtgtacttgcaaatgaatagcttgaaac |
| | | cagaggacactgcaacatactattgcgctgctgccccaaaacaactgaggacgtgt |
| | | ggggattacaactattgggggcaaggcactcaagtcactgtcagttcttccggaacc |
| | | acgacgccagcgccgcgaccaccaacaccggcgcccaccatcgcgtcgcagcc |
| | | cctgtccctgcgcccagaggcgtgccggccagcggcggggggcgcagtgcaca |
| | | cgagggggctggacttcgcctgtgatttttgggtgctggtggtggttggtggagtcct |
| | | ggcttgctatagcttgctagtaacagtggcctttattattttctgggtgaggagtaaga |
| | | ggagcaggctcctgcacagtgactacatgaacatgactccccgccgccccgggcc |
| | | cacccgcaagcattaccagccctatgccccaccacgcgacttcgcagcctatcgct |
| | | ccatcgatagagtgaagttcagcaggagcgcagacgcccccgcgtaccagcagg |
| | | gccagaaccagctctataacgagctcaatctaggacgaagagaggagtacgatgtt |
| | | ttggacaagagacgtggccgggaccctgagatggggggaaagccgagaaggaa |
| | | gaaccctcaggaaggcctgtacaatgaactgcagaaagataagatggcggaggc |
| | | ctacagtgagattgggatgaaaggcgagcgccggaggggcaaggggcacgatg |
| | | gcctttaccagggtctcagtacagccaccaaggacacctacgacgcccttcacatg |
| | | caggccctgccccctcgctaa |
| 26 | 2hD6 nanobody | QVQLQESGGGSVQAGGSLRLSCAASSYTYSYSCMA |
| | amino acid | WFRQAPGKERERVASIHTGTGTANYADSAKGRFTIS |
| | sequence | QDIAANTVYLQMNSLKLEDTAMYYCAARPGSAALR |
| | | CTTDYSKPHDFTYWGQGTQVTVSS |
| 27. | 2hD6 nanobody | caggtccaactccaggaatccggtggtgggtctgtgcaggcagggggcagtctga |
| | nucleic acid | ggttgtcatgtgccgctagttcatacacatactcttactcttgtatggcctggtttcgac |
| | sequence | aggcacccggcaaagagcgggagagagttgccagcattcacacaggaactgga |
| | | accgccaattatgcagattctgctaagggtcggtttacaataagccaggatattgcag |
| | | cgaataccgtatatttgcaaatgaacagccttaagctcgaagatactgcaatgtattac |
| | | tgcgctgcacgaccagggtctgctgcccttaggtgtacgactgattatagcaagccg |
| | | catgattttacttactgggggcaggggacacaggttactgtatcctct |
| 28. | 2hD6 CDR1 | SYTYSYSC |
| | amino acid | |
| | sequence | |
| 29. | 2hD6 CDR2 | IHTGTGTA |
| | amino acid | |
| | sequence | |
| 30. | 2hD6 CDR3 | AARPGSAALRCTTDYSKPHDFTY |
| | amino acid | |
| | sequence | |
| 31. | 2hD6-28z CAR | MALPVTALLLPLALLLHAARPGSQVQLQESGGGSVQ |
| | amino acid | AGGSLRLSCAASSYTYSYSCMAWFRQAPGKERERV |
| | sequence | ASIHTGTGTANYADSAKGRFTISQDIAANTVYLQMN |
| | | SLKLEDTAMYYCAARPGSAALRCTTDYSKPHDFTY |
| | | WGQGTQVTVSSSGTTTPAPRPPTPAPTIASQPLSLRPE |
| | | ACRPAAGGAVHTRGLDFACDFWVLVVVGGVLACYS |
| | | LLVTVAFIIFWVRSKRSRLLHSDYMNMTPRRPGPTRK |
| | | HYQPYAPPRDFAAYRSIDRVKFSRSADAPAYQQGQN |
| | | QLYNELNLGRREEYDVLDKRRGRDPEMGGKPRRKN |
| | | PQEGLYNELQKDKMAEAYSEIGMKGERRRGKGHDG |
| | | LYQGLSTATKDTYDALHMQALPPR |
| 32. | 2hD6-28z CAR | atggccttaccagtgaccgccttgctcctgccgctggccttgctgctccacgccgcc |
| | nucleic acid | aggccgggatcccaggtccaactccaggaatccggtggtgggtctgtgcaggcag |
| | sequence | ggggcagtctgaggttgtcatgtgccgctagttcatacacatactcttactcttgtatg |
| | | gcctggtttcgacaggcacccggcaaagagcgggagagagttgccagcattcaca |
| | | caggaactggaaccgccaattatgcagattctgctaagggtcggtttacaataagcc |
| | | aggatattgcagcgaataccgtatatttgcaaatgaacagccttaagctcgaagatac |
| | | tgcaatgtattactgcgctgcacgaccagggtctgctgcccttaggtgtacgactgat |
| | | tatagcaagccgcatgattttacttactgggggcaggggacacaggttactgtatcct |
| | | cttccggaaccacgacgccagcgccgcgaccaccaacaccggcgcccaccatc |
| | | gcgtcgcagcccctgtccctgcgcccagaggcgtgccggccagcgggggggg |
| | | cgcagtgcacacgagggggctggacttcgcctgtgatttttgggtgctggtggtggt |
| | | tggtggagtcctggcttgctatagcttgctagtaacagtggcctttattattttctgggtg |
| | | aggagtaagaggagcaggctcctgcacagtgactacatgaacatgactccccgcc |
| | | gccccgggcccacccgcaagcattaccagccctatgccccaccacgcgacttcgc |
| | | agcctatcgctccatcgatagagtgaagttcagcaggagcgcagacgcccccgcg |
| | | taccagcagggccagaaccagctctataacgagctcaatctaggacgaagagagg |
| | | agtacgatgttttggacaagagacgtggccgggaccctgagatggggggaaagc |
| | | cgagaaggaagaaccctcaggaaggcctgtacaatgaactgcagaaagataagat |
| | | ggcggaggcctacagtgagattgggatgaaaggcgagcgccggaggggcaag |
| | | gggcacgatggcctttaccagggtctcagtacagccaccaaggacacctacgacg |
| | | cccttcacatgcaggccctgccccctcgctaa |
| 33. | 2hD38 nanobody | QVQLQESGGGSVQAGGSLRLSCAASENTQGNYCLA |
| | amino acid | WFRQAPGKEREGVASISSGGIKTYYADTVKGRFTISR |
| | sequence | DNAENTVYLQMNSLKPEDTAIYYCAARTSVTCFASS |
| | | WARLNAYAYWGQGTQVTVSS |
| 34. | 2hD38 nanobody | caggtgcaacttcaagaatcaggagggggttccgttcaagccggtgggtcactga |
| | nucleic acid | ggctttcttgcgctgcttcagaaaacactcagggcaactactgcctcgcgtggtttcg |
| | sequence | acaagcaccagggaaggaacgggaaggagtggcttccataagcagcggaggaa |
| | | tcaaaacatactacgctgacaccgttaagggccggttcaccatcagccgcgataat |
| | | gccgaaaatactgtgtacttgcagatgaattccctcaaaccagaggatacggctattt |
| | | actactgtgcagcacggaccagcgtgacctgcttcgcctcctcatgggctaggctc |
| | | aacgcttacgcctattggggacagggaactcaggtcacagtaagcagc |
| 35. | 2hD38 CDR1 | ENTQGNYC |
| | amino acid | |
| | sequence | |
| 36. | 2hD38 CDR2 | ISSGGIKT |
| | amino acid | |
| | sequence | |
| 37. | 2hD38 CDR3 | AARTSVTCFASSWARLNAYAY |
| | amino acid | |
| | sequence | |
| 38. | 2hD38-28z CAR | MALPVTALLLPLALLLHAARPGSQVQLQESGGGSVQ |
| | amino acid | AGGSLRLSCAASENTQGNYCLAWFRQAPGKEREGV |
| | sequence | ASISSGGIKTYYADTVKGRFTISRDNAENTVYLQMNS |
| | | LKPEDTAIYYCAARTSVTCFASSWARLNAYAYWGQ |
| | | GTQVTVSSSGTTTPAPRPPTPAPTIASQPLSLRPEACRP |
| | | AAGGAVHTRGLDFACDFWVLVVVGGVLACYSLLVT |
| | | VAFIIFWVRSKRSRLLHSDYMNMTPRRPGPTRKHYQ |
| | | PYAPPRDFAAYRSIDRVKFSRSADAPAYQQGQNQLY |
| | | NELNLGRREEYDVLDKRRGRDPEMGGKPRRKNPQE |
| | | GLYNELQKDKMAEAYSEIGMKGERRRGKGHDGLYQ |
| | | GLSTATKDTYDALHMQALPPR |
| 39. | 2hD38-28z CAR | atggccttaccagtgaccgccttgctcctgccgctggccttgctgctccacgccgcc |
| | nucleic acid | aggccgggatcccaggtgcaacttcaagaatcaggagggggttccgttcaagccg |
| | sequence | gtgggtcactgaggctttcttgcgctgcttcagaaaacactcagggcaactactgcct |
| | | cgcgtggtttcgacaagcaccagggaaggaacgggaaggagtggcttccataag |
| | | cagcggaggaatcaaaacatactacgctgacaccgttaagggccggttcaccatca |
| | | gccgcgataatgccgaaaatactgtgtacttgcagatgaattccctcaaaccagagg |
| | | atacggctatttactactgtgcagcacggaccagcgtgacctgcttcgcctcctcatg |
| | | ggctaggctcaacgcttacgcctattggggacagggaactcaggtcacagtaagc |
| | | agctccggaaccacgacgccagcgccgcgaccaccaacaccggcgcccaccat |
| | | cgcgtcgcagcccctgtccctgcgcccagaggcgtgccggccagcggcggggg |
| | | gcgcagtgcacacgagggggctggacttcgcctgtgatttttgggtgctggtggtg |
| | | gttggtggagtcctggcttgctatagcttgctagtaacagtggcctttattattttctggg |
| | | tgaggagtaagaggagcaggctcctgcacagtgactacatgaacatgactccccg |
| | | ccgccccgggcccacccgcaagcattaccagccctatgccccaccacgcgacttc |
| | | gcagcctatcgctccatcgatagagtgaagttcagcaggagcgcagacgcccccg |
| | | cgtaccagcagggccagaaccagctctataacgagctcaatctaggacgaagaga |
| | | ggagtacgatgttttggacaagagacgtggccgggaccctgagatggggggaaa |
| | | gccgagaaggaagaaccctcaggaaggcctgtacaatgaactgcagaaagataa |
| | | gatggcggaggcctacagtgagattgggatgaaaggcgagcgccggaggggca |
| | | aggggcacgatggcctttaccagggtctcagtacagccaccaaggacacctacga |
| | | cgcccttcacatgcaggccctgccccctcgctaa |
| 63. | huFAP-CAR | atggccttaccagtgaccgccttgctcctgccgctggccttgctgctccacgccgccaggcc |
| | | gggcagcgacatcgtgatgagccagagccccagcagcctggccgtgtccgtgggcgaga |
| | | aagtgaccatgagctgcaagagcagccagagcctgctgtacagccggaaccagaagaac |
| | | tacctggcctggttccagcagaagcccggccagtcccccaagctgctgatcttctgggcca |
| | | gcacccgcgagagcggcgtgccagatagattcaccggcagcggcttcggcaccgacttc |
| | | aacctgaccatcagcagcgtgcaggccgaggatctggccgtgtacgactgccagcagtac |
| | | ttcagctaccccctgaccttcggagccggcaccaagctggaactgaagggcggaggcgg |
| | | ctctggcggcggaggaagttctggcggaggctctgaggtgcagctgcagcagagcggcc |
| | | ctgagctggtgaaacctggcgccagcgtgaagatgtcctgcaagaccagccggtacacctt |
| | | caccgagtacaccatccactgggtgcgccagtctcacggcaagagcctggaatggatcgg |
| | | cggcatcaaccccaacaacggcatccccaactacaaccagaagttcaagggcagagcca |
| | | ccctgaccgtgggcaagtccagcagcaccgcctacatggaactgcggagcctgaccagc |
| | | gaggacagcgctgtgtacttctgcgccagacggcggatcgcctacggctatgacgagggc |
| | | cacgccatggactactggggacagggcaccagcgtgaccgtgtctagctccggaaccac |
| | | gacgccagcgccgcgaccaccaacaccggcgcccaccatcgcgtcgcagcccctgtccc |
| | | tgcgcccagaggcgtgccggccagcggggggggcgcagtgcacacgagggggctgg |
| | | acttcgcctgtgatttttgggtgctggtggtggttggtggagtcctggcttgctatagcttgcta |
| | | gtaacagtggcctttattattttctgggtgaggagtaagaggagcaggctcctgcacagtgac |
| | | tacatgaacatgactccccgccgccccgggcccacccgcaagcattaccagccctatgcc |
| | | ccaccacgcgacttcgcagcctatcgctccatcgatagagtgaagttcagcaggagcgcag |
| | | acgcccccgcgtaccagcagggccagaaccagctctataacgagctcaatctaggacgaa |
| | | gagaggagtacgatgttttggacaagagacgtggccgggaccctgagatggggggaaag |
| | | ccgagaaggaagaaccctcaggaaggcctgtacaatgaactgcagaaagataagatggc |
| | | ggaggcctacagtgagattgggatgaaaggcgagcgccggaggggcaaggggcacgat |
| | | ggcctttaccagggtctcagtacagccaccaaggacacctacgacgcccttcacatgcagg |
| | | ccctgccccctcgctaa |
| 64 | huFAP-F19-L2H | gacatcgtgatgagccagagccccagcagcctggccgtgtccgtgggcgagaaagtgac |
| | scFv | catgagctgcaagagcagccagagcctgctgtacagccggaaccagaagaactacctgg |
| | | cctggttccagcagaagcccggccagtcccccaagctgctgatcttctgggccagcacccg |
| | | cgagagcggcgtgccagatagattcaccggcagcggcttcggcaccgacttcaacctgac |
| | | catcagcagcgtgcaggccgaggatctggccgtgtacgactgccagcagtacttcagctac |
| | | cccctgaccttcggagccggcaccaagctggaactgaagggcggaggcggctctggcgg |
| | | cggaggaagttctggcggaggctctgaggtgcagctgcagcagagcggccctgagctgg |
| | | tgaaacctggcgccagcgtgaagatgtcctgcaagaccagccggtacaccttcaccgagta |
| | | caccatccactgggtgcgccagtctcacggcaagagcctggaatggatcggcggcatcaa |
| | | ccccaacaacggcatccccaactacaaccagaagttcaagggcagagccaccctgaccgt |
| | | gggcaagtccagcagcaccgcctacatggaactgcggagcctgaccagcgaggacagc |
| | | gctgtgtacttctgcgccagacggcggatcgcctacggctatgacgagggccacgccatg |
| | | gactactggggacagggcaccagcgtgaccgtgtctagc |

Accordingly, a subject CAR may be a CAR having affinity for DPP6, comprising an DPP6 binding domain comprising an amino acid sequence set forth in SEQ ID NOs: 1, 18, 27, and 34. A subject DPP6 CAR may further comprise a hinge domain comprising an amino acid sequence set forth in SEQ ID NO: 22. A subject DPP6 CAR may further comprise a human IgG4 spacer domain comprising an amino acid sequence set forth in SEQ ID NO: 7 A subject DPP6 CAR may further comprise a transmembrane domain comprising an amino acid sequence set forth in SEQ ID NO: 10. A subject DPP6 CAR may further comprise a CD28 intracellular domain comprising an amino acid sequence set forth in SEQ ID NO: 11. A subject DPP6 CAR may comprise a CD3 intracellular domain comprising an amino acid sequence set forth in SEQ ID NO: 13. A subject DPP6 CAR may comprise an amino acid sequence set forth in SEQ ID NOs: 15, 24, 31, and 38.

### Antigen Binding Domain

The antigen binding domain of a CAR is an extracellular region of the CAR for binding to a specific target antigen including proteins, carbohydrates, and glycolipids. In some embodiments, the CAR comprises affinity to a target antigen on a target cell. The target antigen may include any type of protein, or epitope thereof, associated with the target cell. For example, the CAR may comprise affinity to a target antigen on a target cell that indicates a particular status, cell type or tissue location of the target cell.

In one embodiment, the CAR of the invention comprises an antigen binding domain that binds to DPP6 or FAP. In another embodiment, the antigen binding domain of the invention comprises an antibody or fragment thereof, that binds to an DPP6 or FAP molecule. Preferably, the antigen binding domain is a nanobody that binds to an DPP6 molecule/epitope. In other preferred embodiments, the antigen binding domain is a scFv or single-chain variable fragment that binds to an FAP molecule/epitope. The choice of antigen binding domain depends upon the type and number of antigens that are present on the surface of a target cell. For example, the antigen binding domain may be chosen to recognize an antigen that acts as a cell surface marker on a target cell associated with a particular status, function, or identity of the target cell.

In some embodiments, a CAR of the present disclosure may have affinity for one or more target antigens on one or more target cells. In some embodiments, a CAR may have affinity for one or more target antigens on a target cell. In such embodiments, the CAR is a bispecific CAR, or a multispecific CAR. In some embodiments, the CAR comprises one or more target-specific binding domains that confer affinity for one or more target antigens. In some embodiments, the CAR comprises one or more target-specific binding domains that confer affinity for the same target antigen. For example, a CAR comprising one or more target-specific binding domains having affinity for the same target antigen could bind distinct epitopes of the target antigen. When a plurality of target-specific binding domains is present in a CAR, the binding domains may be arranged in tandem and may be separated by linker peptides. For example, in a CAR comprising two target-specific binding domains, the binding domains are connected to each other covalently on a single polypeptide chain, through an oligo- or polypeptide linker, an Fc hinge region, or a membrane hinge region.

The antigen binding domain can include any domain that binds to the antigen and may include, but is not limited to, a monoclonal antibody, a polyclonal antibody, a synthetic antibody, a nanobody, and any fragment thereof. Thus, in one embodiment, the antigen binding domain portion comprises a single-domain nanobody or a fragment thereof. In another embodiment, the antigen binding domain of the CAR is selected from the group consisting of an anti-DPP6 antibody or a fragment thereof. In some embodiments, a DPP6 binding domain of the present invention is selected from the group consisting of a DPP6-specific antibody, a DPP6-specific Fab, a DPP6-specific nanobody, and a DPP6-specific scFv. In one embodiment, a DPP6 binding domain is a DPP6-specific nanobody. In another embodiment, the antigen binding domain of the CAR is selected from the group consisting of an anti-FAP antibody or a fragment thereof. In some embodiments, a FAP binding domain of the present invention is selected from the group consisting of a FAP-specific antibody, a FAP-specific Fab, a FAP-specific nanobody, and a FAP-specific scFv. In one embodiment, a FAP binding domain is a FAP-specific scFv.

A “nanobody”, as used herein, refers to the smallest antigen binding fragment or single variable domain (“VHH”) derived from naturally occurring heavy chain antibody.

Nanobodies are derived from heavy chain only antibodies, produced by camelids (Hamers-Casterman et al. 1993; Desmyter et al. 1996). “Camelids” comprise old world camelids (Camelus bactrianus and Camelus dromedarius) and new world camelids (for example Lama paccos, Lama glama, Lama guanicoe and Lama vicugna). Single variable domain heavy chain antibody is herein designated as a nanobody or a VHH antibody Nanobody™, Nanobodies™ and Nanoclone™ are trademarks of AblynxNV (Belgium). The small size and unique biophysical properties of nanobodies are often superior to conventional heavy chain/light chain antibody fragments for the recognition of uncommon or obstructed epitopes and for binding into cavities or active sites of protein targets. Further, nanobodies can be designed as bispecific and bivalent antibodies or attached to other molecules including polypeptides, reporter molecules (Conrath et al. 2001), or drug conjugates. In one embodiment, the DPP6-specific antigen binding domains of the CARs of the current invention are derived from nanobodies of camelid origin.

As used herein, the term “single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker or spacer, which connects the N-terminus of the VH with the C-terminus of the VL, or the C-terminus of the VH with the N-terminus of the VL. Those of skill in the art would be able to select the appropriate configuration for use. In one embodiment, the FAP-specific antigen binding domains of the CARs of the current invention are derived from scFvs.

The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers such as (GS)ₙ, (GSGGS)ₙ (SEQ ID NO: 40), (GGGS)ₙ (SEQ ID NO: 41), and (GGGGS)ₙ(SEQ ID NO: 42), where n represents an integer of at least 1. Exemplary linker sequences can comprise amino acid sequences including, without limitation, GGSG (SEQ ID NO: 43), GGSGG (SEQ ID NO: 44), GSGSG (SEQ ID NO: 45), GSGGG (SEQ ID NO: 46), GGGSG (SEQ ID NO: 47), GSSSG (SEQ ID NO: 48), GGGGS (SEQ ID NO: 49), GGGGSGGGGSGGGGS (SEQ ID NO: 50) and the like.

Those of skill in the art would be able to select the appropriate linker sequence for use in the present invention. In one embodiment, an antigen binding domain (e.g., DPP6 binding domain) of the present invention comprises a heavy chain variable region (VH) and a light chain variable region (VL), wherein the VH and VL is separated by the linker sequence having the amino acid sequence GGGGSGGGGSGGGGS (SEQ ID NO: 50), which may be encoded by the nucleic acid sequence ggtggcggtggctcgggcggtggtgggtcgggt ggcggcggatct (SEQ ID NO: 51).

Despite removal of the constant regions and the introduction of a linker, scFv proteins retain the specificity of the original immunoglobulin. Single chain FAT polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 2008 27(6):455-51; Peter et al., J Cachexia Sarcopenia Muscle 2012 August 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In an exemplary embodiment, an DPP6 CAR of the present invention comprises an DPP6 binding domain, e.g., an DPP6-specific nanobody. In one embodiment, the DPP6 binding domain comprises the amino acid sequence set forth in SEQ ID NOs: 1, 17, 26, and 33.

The DPP6 binding domain comprises three complementarity-determining regions (CDRs). As used herein, a “complementarity-determining region” or “CDR” refers to a region of the variable chain of an antigen binding molecule that binds to a specific antigen. Accordingly, an DPP6 binding domain may comprise a CDR1 comprising an amino acid sequence set forth in SEQ ID NO: 3; a CDR2 comprising an amino acid sequence set forth in SEQ ID NO: 4; and a CDR3 comprising the amino acid sequence AT.

Tolerable variations of the DPP6 binding domain will be known to those of skill in the art, while maintaining specific binding to DPP6. For example, in some embodiments the DPP6 binding domain comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NOs: 1, 17, 26, and 33. For example, in some embodiments the DPP6 binding domain is encoded by a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the nucleic acid sequences set forth in 2, 18, 27, and 34.

The antigen binding domain may be operably linked to another domain of the CAR, such as the transmembrane domain or the intracellular domain, both described elsewhere herein. In one embodiment, a nucleic acid encoding the antigen binding domain is operably linked to a nucleic acid encoding a transmembrane domain and a nucleic acid encoding one or more intracellular domains.

The antigen binding domains described herein, such as the DPP6 binding domain, can be combined with any of the transmembrane domains described herein, any of the intracellular domains or cytoplasmic domains described herein, or any of the other domains described herein that may be included in the CAR.

### Transmembrane Domain

With respect to the transmembrane domain, the CAR of the present invention (e.g., DPP6 CAR) can be designed to comprise a transmembrane domain that connects the antigen binding domain of the CAR to the intracellular domain. The transmembrane domain of a subject CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen binding domain and the intracellular domain of a CAR.

In one embodiment, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane regions of particular use in this invention include, without limitation, transmembrane domains derived from (i.e. comprise at least the transmembrane region(s) of) the alpha, beta or zeta chain of the T-cell receptor, CD28, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), Toll-like receptor 1 (TLR1), TLR2, TLR3, TLR4, TLR5, TLR6, TLR7, TLR8, and TLR9. In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

The transmembrane domains described herein can be combined with any of the antigen binding domains described herein, any of the intracellular domains described herein, or any of the other domains described herein that may be included in a subject CAR.

In some embodiments, the transmembrane domain further comprises a hinge or spacer region. A subject CAR of the present invention may also include a hinge or spacer region. The hinge or spacer region of the CAR is a hydrophilic region which is located between the antigen binding domain and the transmembrane domain. In some embodiments, this domain facilitates proper protein folding for the CAR. The hinge or spacer region is an optional component for the CAR. The hinge or spacer region may include a domain selected from Fc fragments of antibodies, hinge or spacer regions of antibodies, CH2 regions of antibodies, CH3 regions of antibodies, artificial hinge sequences or combinations thereof. Examples of hinge and spacer regions include, without limitation, a CD8a hinge, artificial hinges made of polypeptides which may be as small as, three glycines (Gly), as well as CH1 and CH3 domains of IgGs (such as human IgG4).

In some embodiments, a subject CAR of the present disclosure includes a hinge or spacer region that connects the antigen binding domain with the transmembrane domain, which, in turn, connects to the intracellular domain. The hinge or spacer region is preferably capable of supporting the antigen binding domain to recognize and bind to the target antigen on the target cells (see, e.g., Hudecek et al., Cancer Immunol. Res. (2015) 3(2): 125-135). In some embodiments, the hinge or spacer region is a flexible domain, thus allowing the antigen binding domain to have a structure to optimally recognize the specific structure and density of the target antigens on a cell such as tumor cell (Hudecek et al., supra). The flexibility of the hinge or spacer region permits the hinge region to adopt many different conformations.

In some embodiments, the hinge or spacer region is an immunoglobulin heavy chain hinge region. In some embodiments, the hinge or spacer region is a hinge region polypeptide derived from a receptor (e.g., a CD8-derived hinge region).

The hinge or spacer region can have a length of from about 4 amino acids to about 300 amino acids. Suitable hinge or spacer regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

For example, hinge regions include glycine polymers (G)ₙ, glycine-serine polymers (including, for example, (GS)ₙ, (GSGGS)ₙ (SEQ ID NO: 40) and (GGGS)ₙ (SEQ ID NO: 41), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). Exemplary hinge regions can comprise amino acid sequences including, but not limited to, GGSG (SEQ ID NO: 43), GGSGG (SEQ ID NO: 44), GSGSG (SEQ ID NO: 45), GSGGG (SEQ ID NO: 46), GGGSG (SEQ ID NO: 47), GSSSG (SEQ ID NO: 48), and the like.

In some embodiments, the hinge region is an immunoglobulin heavy chain hinge region. Immunoglobulin hinge region amino acid sequences are known in the art; see, e.g., Tan et al., Proc. Natl. Acad. Sci. USA (1990) 87(1):162-166; and Huck et al., Nucleic Acids Res. (1986) 14(4): 1779-1789. As non-limiting examples, an immunoglobulin hinge region can include one of the following amino acid sequences: DKTHT (SEQ ID NO: 52); CPPC (SEQ ID NO: 53); CPEPKSCDTPPPCPR (SEQ ID NO: 54) (see, e.g., Glaser et al., J. Biol. Chem. (2005) 280:41494-41503); ELKTPLGDTTHT (SEQ ID NO: 55); KSCDKTHTCP (SEQ ID NO: 56); KCCVDCP (SEQ ID NO: 57); KYGPPCP (SEQ ID NO: 58); EPKSCDKTHTCPPCP (SEQ ID NO: 59) (human IgG1 hinge); ERKCCVECPPCP (SEQ ID NO: 60) (human IgG2 hinge); ELKTPLGDTTHTCPRCP (SEQ ID NO: 61) (human IgG3 hinge); SPNMVPHAHHAQ (SEQ ID NO: 62) (human IgG4 hinge); and the like.

The hinge or spacer region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO: 59); see, e.g., Yan et al., J. Biol. Chem. (2012) 287: 5891-5897. In one embodiment, the hinge region can comprise an amino acid sequence derived from human CD8, or a variant thereof. In one embodiment, the hinge region is derived from CD8 and comprises the amino acid sequence set forth in SEQ ID NO: 22 and is encoded by the nucleic acid sequence set forth in SEQ ID NO: 23. In another embodiment, the hinge or spacer region is derived from human IgG4 and comprises the amino acid sequence set forth in SEQ ID NO: 7 and is encoded by the nucleic acid sequence set forth in SEQ ID NO: 8.

In one embodiment, the transmembrane domain comprises a CD28 transmembrane domain. In another embodiment, the transmembrane domain comprises a CD8 hinge domain and a CD28 transmembrane domain. In some embodiments, a subject CAR comprises a CD8 hinge region having the amino acid sequence set forth in SEQ ID NO: 22, which may be encoded by the nucleic acid sequence set forth in SEQ ID NO: 23. In some embodiments, a subject CAR comprises a IgG4 spacer region having the amino acid sequence set forth in SEQ ID NO: 7, which may be encoded by the nucleic acid sequence set forth in SEQ ID NO: 8. In some embodiments, a subject CAR comprises a CD28 transmembrane domain having the amino acid sequence set forth in SEQ ID NO: 10, which may be encoded by the nucleic acid sequence set forth in SEQ ID NO: 9. In some embodiments, the transmembrane domain comprises a CD8 hinge region and a CD28 transmembrane domain. In some embodiments, the transmembrane domain comprises a IgG4 spacer region and a CD28 transmembrane domain.

Tolerable variations of the transmembrane and/or hinge or spacer domain will be known to those of skill in the art, while maintaining its intended function. For example, in some embodiments the hinge domain and/or transmembrane domain comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NOs: 7, 10, and/or 22. For example, in some embodiments the hinge or spacer domain and/or transmembrane domain is encoded by a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the nucleic acid sequences set forth in SEQ ID NOs: 8, 9, and/or 23.

The transmembrane domain may be combined with any hinge or spacer domain and/or may comprise one or more transmembrane domains described herein.

The transmembrane domains described herein, such as a transmembrane region of alpha, beta or zeta chain of the T-cell receptor, CD28, CD3 epsilon, CD45, CD4, CDS, CD7, CD8, CD9, CD 16, CD22, CD33, CD37, CD64, CD80, CD86, CD134 (OX-40), CD137 (4-1BB), CD154 (CD40L), Toll-like receptor 1 (TLR1), TLR2, TLR3, TLR4, TLR5, TLR6, TLR7, TLR8, and TLR9, can be combined with any of the antigen binding domains described herein, any of the intracellular domains or cytoplasmic domains described herein, or any of the other domains described herein that may be included in the CAR.

In one embodiment, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

As used herein, the term “spacer domain” generally means any oligo- or polypeptide that functions to link the transmembrane domain to, either the extracellular domain or, the intracellular domain in the polypeptide chain. A spacer domain may comprise up to 300 amino acids, e.g., 10 to 100 amino acids, or 25 to 50 amino acids. In some embodiments, the spacer domain may be a short oligo- or polypeptide linker, e.g., between 2 and 10 amino acids in length. For example, glycine-serine doublet provides a particularly suitable linker between the transmembrane domain and the intracellular signaling domain of the subject CAR.

### Intracellular Domain

A subject CAR of the present invention also includes an intracellular signaling domain. The terms “intracellular signaling domain” and “intracellular domain” are used interchangeably herein. The intracellular signaling domain of the CAR is responsible for activation of at least one of the effector functions of the cell in which the CAR is expressed (e.g., immune cell). The intracellular signaling domain transduces the effector function signal and directs the cell (e.g., immune cell) to perform its specialized function, e.g., harming and/or destroying a target cell.

The intracellular domain or otherwise the cytoplasmic domain of the CAR is responsible for activation of the cell in which the CAR is expressed. In one embodiment, the intracellular domain comprises CD3 zeta. In another embodiment, the intracellular domain comprises CD28 and CD3 zeta.

Examples of an intracellular domain for use in the invention include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

Examples of the intracellular signaling domain include, without limitation, the ζ chain of the T cell receptor complex or any of its homologs, e.g., η chain, FcsRIγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain, FcγRIII, FcsRI, cytoplasmic tails of Fc receptors, an immunoreceptor tyrosine-based activation motif (ITAM) bearing cytoplasmic receptors, and combinations thereof.

In one embodiment, the intracellular domain of the CAR includes any portion of one or more co-stimulatory molecules, such as at least one signaling domain from CD3, CD8, CD27, CD28, ICOS, 4-IBB, PD-1, any derivative or variant thereof, any synthetic sequence thereof that has the same functional capability, and any combination thereof.

Other examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fcgamma Rlla, DAP10, DAP 12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD 160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1 Id, ITGAE, CD 103, ITGAL, CD 11 a, LFA-1, ITGAM, CD lib, ITGAX, CD 11c, ITGBl, CD29, ITGB2, CD 18, LFA-1, ITGB7, TNFR2, TRANCE/RANKL, DNAM1 (CD226), SLAMF4 (CD244, 2B4), CD84, CD 96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in a subject CAR of the present invention include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in a subject CAR of the present invention include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of a subject CAR comprises 3 ITAM motifs. In some embodiments, intracellular signaling domains includes the signaling domains of human immunoglobulin receptors that contain immunoreceptor tyrosine based activation motifs (ITAMs) such as, but not limited to, FcgammaRI, FcgammaRllA, FcgammaRllC, FcgammaRIIIA, FcRL5 (see, e.g., Gillis et al., Front. Immunol. (2014) 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcRgamma; fceRl gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In one embodiment, an intracellular signaling domain suitable for use in an FN3 CAR of the present disclosure includes a DAP10/CD28 type signaling chain. In one embodiment, an intracellular signaling domain suitable for use in an FN3 CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CDS, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the antigen binding domains described herein, any of the transmembrane domains described herein, or any of the other domains described herein that may be included in the CAR.

In one embodiment, the intracellular domain of a subject CAR comprises a CD28 intracellular domain comprising the amino acid sequence set forth in SEQ ID NO: 11, which may be encoded by the nucleic acid sequence set forth in SEQ ID NO: 12. In one embodiment, the intracellular domain of a subject CAR comprises a CD3 zeta domain comprising the amino acid sequence set forth in SEQ ID NO: 13, which may be encoded by the nucleic acid sequence set forth in SEQ ID NO: 14. In one exemplary embodiment, the intracellular domain of a subject CAR comprises a CD28 domain and a CD3 zeta domain.

Tolerable variations of the intracellular domain will be known to those of skill in the art, while maintaining specific activity. For example, in some embodiments the intracellular domain comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NOs: 11 and/or 13. For example, in some embodiments the intracellular domain is encoded by a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the nucleic acid sequences set forth in SEQ ID NOs: 12 and/or 14.

### CAR Sequences

A subject CAR of the present invention may be a CAR having affinity for DPP6. In one embodiment, the DPP6 CAR of the present invention comprises the amino acid sequence set forth in SEQ ID NOs: 15, 24, 31, and/or 38, which may be encoded by the nucleic acid sequence set forth in SEQ ID NOs: 16, 25, 32, and/or 39. In another embodiment, the subject CAR of the present invention may be a CAR having affinity for FAP. In one embodiment, the FAP CAR of the present invention is encoded by the nucleic acid sequence set forth in SEQ ID NO: 63.

Tolerable variations of the CAR will be known to those of skill in the art, while maintaining specific activity. For example, in some embodiments the CAR comprises an amino acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to the amino acid sequence set forth in SEQ ID NOs: 15, 24, 31, and/or 39. For example, in some embodiments the CAR is encoded by a nucleic acid sequence that has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to the nucleic acid sequence set forth in SEQ ID NOs: 16, 25, 32, 39, and/or 63. Accordingly, a subject CAR of the present invention comprises an DPP6 binding domain and a transmembrane domain. In one embodiment, the CAR comprises an DPP6 binding domain and a transmembrane domain, wherein the transmembrane domain comprises a CD8 hinge region. In one embodiment, the CAR comprises an DPP6 binding domain and a transmembrane domain, wherein the transmembrane domain comprises a IgG4 spacer domain. In one embodiment, the CAR comprises an DPP6 binding domain and a transmembrane domain, wherein the transmembrane domain comprises a CD28 transmembrane domain. In one embodiment, the CAR comprises an DPP6 binding domain and a transmembrane domain, wherein the transmembrane domain comprises a CD8 hinge region and a CD28 transmembrane domain. In one embodiment, the CAR comprises an DPP6 binding domain and a transmembrane domain, wherein the transmembrane domain comprises a IgG4 spacer domain and a CD28 transmembrane domain.

Accordingly, a subject CAR of the present invention comprises an DPP6 binding domain, a transmembrane domain, and an intracellular domain. In one embodiment, the CAR comprises an DPP6 binding domain, a transmembrane domain, and an intracellular domain, wherein the intracellular domain comprises a CD28 domain. In one embodiment, the CAR comprises an DPP6 binding domain, a transmembrane domain, and an intracellular domain, wherein the intracellular domain comprises a CD3 zeta domain. In one embodiment, the CAR comprises an DPP6 binding domain, a transmembrane domain, and an intracellular domain, wherein the intracellular domain comprises a CD28 domain and a CD3 zeta domain.

Accordingly, a subject CAR of the present invention comprises an DPP6 binding domain, a CD8 hinge region, a CD28 transmembrane domain, a CD28 intracellular domain, and a CD3 zeta intracellular domain.

Accordingly, a subject CAR of the present invention comprises an DPP6 binding domain, a IgG4 spacer region, a CD28 transmembrane domain, a CD28 intracellular domain, and a CD3 zeta intracellular domain.

Accordingly, the present invention provides a modified immune cell or precursor cell thereof, e.g., a modified regulatory T cell, comprising a chimeric antigen receptor (CAR) having affinity for DPP6 as described herein. Accordingly, a subject CAR of the present invention comprises an FAP binding domain, a transmembrane domain, and an intracellular domain. In one embodiment, the CAR comprises an FAP binding domain, a transmembrane domain, and an intracellular domain, wherein the intracellular domain comprises a CD28 domain. In one embodiment, the CAR comprises an FAP binding domain, a transmembrane domain, and an intracellular domain, wherein the intracellular domain comprises a CD3 zeta domain. In one embodiment, the CAR comprises an FAP binding domain, a transmembrane domain, and an intracellular domain, wherein the intracellular domain comprises a CD28 domain and a CD3 zeta domain.

Accordingly, a subject CAR of the present invention comprises an FAP binding domain, a CD8 hinge region, a CD28 transmembrane domain, a CD28 intracellular domain, and a CD3 zeta intracellular domain.

Accordingly, the present invention provides a modified immune cell or precursor cell thereof, e.g., a modified regulatory T cell, comprising a chimeric antigen receptor (CAR) having affinity for FAP as described herein.

### Nucleic Acids and Expression Vectors

The present invention provides a nucleic acid encoding a CAR having affinity for DPP6. As described herein, a subject CAR comprises an antigen binding domain (e.g., DPP6 binding domain), a transmembrane domain, and an intracellular domain. Accordingly, the present invention provides a nucleic acid encoding an antigen binding domain (e.g., DPP6 binding domain), a transmembrane domain, and an intracellular domain of a subject CAR.

In an exemplary embodiment, a nucleic acid encoding an DPP6 CAR of the present invention is encoded by a nucleic acid sequence set forth in SEQ ID NOs: 16, 25, 32, and/or 39.

In some embodiments, a nucleic acid of the present disclosure may be operably linked to a transcriptional control element, e.g., a promoter, and enhancer, etc. Suitable promoter and enhancer elements are known to those of skill in the art.

For expression in a bacterial cell, suitable promoters include, but are not limited to, lad, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (A1cR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some embodiments, the promoter is a CD8 cell-specific promoter, a CD4 cell-specific promoter, a neutrophil-specific promoter, or an NK-specific promoter. For example, a CD4 gene promoter can be used; see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter; see, e.g., Eckelhart et al. Blood (2011) 117:1565.

For expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al., Infect. Immun. (2002) 70:1087-1096); an rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (Lad repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, the EF-1 alpha promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired.

Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the disclosures of which are incorporated herein by reference.

In some embodiments, a nucleic acid of the present disclosure further comprises a nucleic acid sequence encoding a CAR inducible expression cassette. In one embodiment, the CAR inducible expression cassette is for the production of a transgenic polypeptide product that is released upon CAR signaling. See, e.g., Chmielewski and Abken, Expert Opin. Biol. Ther. (2015) 15(8): 1145-1154; and Abken, Immunotherapy (2015) 7(5): 535-544.

A nucleic acid of the present disclosure may be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example, and should not be construed in any way as limiting: Bacterial: pBs, phagescript, PsiX174, pBluescript SK, pBs KS, pNH8a, pNH16a, pNH18a, pNH46a (Stratagene, La Jolla, Calif, USA); pTrc99A, pKK223-3, pKK233-3, pDR540, and pRITS (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35: 2543-2549; Borras et al., Gene Ther. (1999) 6: 515-524; Li and Davidson, Proc. Natl. Acad. Sci. USA (1995) 92: 7700-7704; Sakamoto et al., H. Gene Ther. (1999) 5: 1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9: 81-86, Flannery et al., Proc. Natl. Acad. Sci. USA (1997) 94: 6916-6921; Bennett et al., Invest. Opthalmol. Vis. Sci. (1997) 38: 2857-2863; Jomary et al., Gene Ther. (1997) 4:683 690, Rolling et al., Hum. Gene Ther. (1999) 10: 641-648; Ali et al., Hum. Mol. Genet. (1996) 5: 591-594; Srivastava in WO 93/09239, Samulski et al., J. Vir. (1989) 63: 3822-3828; Mendelson et al., Virol. (1988) 166: 154-165; and Flotte et al., Proc. Natl. Acad. Sci. USA (1993) 90: 10613-10617); SV40; herpes simplex virus; human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector (e.g., a lentiviral vector) may be used to introduce the CAR into an immune cell or precursor thereof (e.g., a T cell). Accordingly, an expression vector (e.g., a lentiviral vector) of the present invention may comprise a nucleic acid encoding a CAR. In some embodiments, the expression vector (e.g., lentiviral vector) will comprise additional elements that will aid in the functional expression of the CAR encoded therein. In some embodiments, an expression vector comprising a nucleic acid encoding a CAR further comprises a mammalian promoter. In one embodiment, the vector further comprises an elongation-factor-1-alpha promoter (EF-1α promoter). Use of an EF-1α promoter may increase the efficiency in expression of downstream transgenes (e.g., a CAR encoding nucleic acid sequence). Physiologic promoters (e.g., an EF-1α promoter) may be less likely to induce integration mediated genotoxicity, and may abrogate the ability of the retroviral vector to transform stem cells. Other physiological promoters suitable for use in a vector (e.g., lentiviral vector) are known to those of skill in the art and may be incorporated into a vector of the present invention. In some embodiments, the vector (e.g., lentiviral vector) further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and may be incorporated into a vector (e.g., lentiviral vector) of the present invention. In some embodiments, the vector further comprises a posttranscriptional regulatory element. Posttranscriptional regulatory elements may improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present invention further comprises a WPRE sequence. Various posttranscriptional regulator elements are known to those of skill in the art and may be incorporated into a vector (e.g., lentiviral vector) of the present invention. A vector of the present invention may further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) of the present invention includes a 3′ U3 deleted LTR. Accordingly, a vector (e.g., lentiviral vector) of the present invention may comprise any combination of the elements described herein to enhance the efficiency of functional expression of transgenes. For example, a vector (e.g., lentiviral vector) of the present invention may comprise a WPRE sequence, cPPT sequence, RRE sequence, 5′LTR, 3′ U3 deleted LTR′ in addition to a nucleic acid encoding for a CAR.

Vectors of the present invention may be self-inactivating vectors. As used herein, the term “self-inactivating vector” refers to vectors in which the 3′ LTR enhancer promoter region (U3 region) has been modified (e.g., by deletion or substitution). A self-inactivating vector may prevent viral transcription beyond the first round of viral replication. Consequently, a self-inactivating vector may be capable of infecting and then integrating into a host genome (e.g., a mammalian genome) only once, and cannot be passed further. Accordingly, self-inactivating vectors may greatly reduce the risk of creating a replication-competent virus.

In some embodiments, a nucleic acid of the present invention may be RNA, e.g., in vitro synthesized RNA. Methods for in vitro synthesis of RNA are known to those of skill in the art; any known method can be used to synthesize RNA comprising a sequence encoding a CAR of the present disclosure. Methods for introducing RNA into a host cell are known in the art. See, e.g., Zhao et al. Cancer Res. (2010) 15: 9053. Introducing RNA comprising a nucleotide sequence encoding a CAR of the present disclosure into a host cell can be carried out in vitro or ex vivo or in vivo. For example, a host cell (e.g., an NK cell, a cytotoxic T lymphocyte, etc.) can be electroporated in vitro or ex vivo with RNA comprising a nucleotide sequence encoding a CAR of the present disclosure.

In order to assess the expression of a polypeptide or portions thereof, the expression vector to be introduced into a cell may also contain either a selectable marker gene or a reporter gene, or both, to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

### Methods of Generating Modified Immune Cells

The present invention provides methods for producing/generating a modified immune cell or precursor cell thereof (e.g., a regulatory T cell). The cells are generally engineered by introducing a nucleic acid encoding a subject CAR (e.g., DPP6 CAR).

Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

In some embodiments, a nucleic acid encoding a subject CAR of the invention is introduced into a cell by an expression vector. Expression vectors comprising a nucleic acid encoding a subject CAR (e.g., DPP6 or FAP CAR) are provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggybak, and Integrases such as Phi31. Some other suitable expression vectors include Herpes simplex virus (HSV) and retrovirus expression vectors. Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the subject CAR in the host cell. In some embodiments, the adenovirus genome is a 36 kb, linear, double stranded DNA, where a foreign DNA sequence (e.g., a nucleic acid encoding a subject CAR) may be inserted to substitute large pieces of adenoviral DNA in order to make the expression vector of the present invention (see, e.g., Danthinne and Imperiale, Gene Therapy (2000) 7(20): 1707-1714).

Another expression vector is based on an adeno associated virus, which takes advantage of the adenovirus coupled systems. This AAV expression vector has a high frequency of integration into the host genome. It can infect non-dividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797,368.

Retrovirus expression vectors are capable of integrating into the host genome, delivering a large amount of foreign genetic material, infecting a broad spectrum of species and cell types and being packaged in special cell lines. The retrovirus vector is constructed by inserting a nucleic acid (e.g., a nucleic acid encoding a subject CAR) into the viral genome at certain locations to produce a virus that is replication defective. Though the retrovirus vectors are able to infect a broad variety of cell types, integration and stable expression of the subject CAR, requires the division of host cells.

Lentivirus vectors are derived from lentiviruses, which are complex retroviruses that, in addition to the common retroviral genes gag, pol, and env, contain other genes with regulatory or structural function (see, e.g., U.S. Pat. Nos. 6,013,516 and 5,994,136). Some examples of lentiviruses include the Human Immunodeficiency Viruses (HIV-1, HIV-2) and the Simian Immunodeficiency Virus (SIV). Lentivirus vectors have been generated by multiply attenuating the HIV virulence genes, for example, the genes env, vif, vpr, vpu and nef are deleted making the vector biologically safe. Lentivirus vectors are capable of infecting non-dividing cells and can be used for both in vivo and ex vivo gene transfer and expression, e.g., of a nucleic acid encoding a subject CAR (see, e.g., U.S. Pat. No. 5,994,136).

Expression vectors including a nucleic acid of the present disclosure can be introduced into a host cell by any means known to persons skilled in the art. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. The host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell is an immune cell or precursor thereof, e.g., a T cell, an NK cell, or an NKT cell.

The present invention also provides genetically engineered cells which include and stably express a subject CAR of the present disclosure. In some embodiments, the genetically engineered cells are genetically engineered T-lymphocytes (T cells), regulatory T cells (Tregs), naive T cells (TN), memory T cells (for example, central memory T cells (TCM), effector memory cells (TEM)), natural killer cells (NK cells), and macrophages capable of giving rise to therapeutically relevant progeny. In one embodiment, the genetically engineered cells are autologous cells.

Modified cells (e.g., comprising a subject CAR) may be produced by stably transfecting host cells with an expression vector including a nucleic acid of the present disclosure. Additional methods to generate a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells expressing a subject CAR of the present disclosure may be expanded ex vivo.

Physical methods for introducing an expression vector into host cells include calcium phosphate precipitation, lipofection, particle bombardment, microinjection, electroporation, and the like. Methods for producing cells including vectors and/or exogenous nucleic acids are well-known in the art. See, e.g., Sambrook et al. (2001), Molecular Cloning: A Laboratory Manual, Cold Spring Harbor Laboratory, New York.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY);

cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the nucleic acids in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Moreover, the nucleic acids may be introduced by any means, such as transducing the expanded T cells, transfecting the expanded T cells, and electroporating the expanded T cells. One nucleic acid may be introduced by one method and another nucleic acid may be introduced into the T cell by a different method.

### RNA

In one embodiment, the nucleic acids introduced into the host cell are RNA. In another embodiment, the RNA is mRNA that comprises in vitro transcribed RNA or synthetic RNA. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA.

PCR can be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary”, as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

In some embodiments, the RNA is electroporated into the cells, such as in vitro transcribed RNA.

The disclosed methods can be applied to the modulation of host cell activity in basic research and therapy, in the fields of cancer, stem cells, acute and chronic infections, and autoimmune diseases, including the assessment of the ability of the genetically modified host cell to kill a target cancer cell.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

One advantage of RNA transfection methods of the invention is that RNA transfection is essentially transient and a vector-free. A RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of host cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

RNA has several advantages over more traditional plasmid or viral approaches. Gene expression from an RNA source does not require transcription and the protein product is produced rapidly after the transfection. Further, since the RNA has to only gain access to the cytoplasm, rather than the nucleus, and therefore typical transfection methods result in an extremely high rate of transfection. In addition, plasmid based approaches require that the promoter driving the expression of the gene of interest be active in the cells under study.

In another aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and

U.S. Pat. No. 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

### Sources of Immune Cells

Prior to expansion, a source of immune cells is obtained from a subject for ex vivo manipulation. Sources of target cells for ex vivo manipulation may also include, e.g., autologous or heterologous donor blood, cord blood, or bone marrow. For example, the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. Preferably, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell, a hematopoietic stem cell, a natural killer cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product.

Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some aspects, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune cells are obtained from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker) or express relatively low levels (marker^{low}) of one or more markers. For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In some examples, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. In some embodiments, combining TCM-enriched CD8+ T cells and CD4+ T cells further enhances efficacy.

In some embodiments, memory T cells are present in both CD62L+ and CD62L-subsets of CD8+ peripheral blood lymphocytes. PBMC can be enriched for or depleted of CD62L-CD8+ and/or CD62L+CD8+ fractions, such as using anti-CD8 and anti-CD62L antibodies. In some embodiments, a CD4+ T cell population and/or a CD8+ T population is enriched for central memory (TCM) cells. In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+ T helper cells are sorted into naive, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO. In one example, to enrich for CD4+ cells by negative selection, a monoclonal antibody cocktail typically includes antibodies to CD14, CD20, CDl lb, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

In certain embodiments, T regulatory cells (Tregs) can be isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. Methods for isolating Tregs are described in U.S. Pat. Nos. 7,754,482, 8,722,400, and 9,555,105, and U.S. patent application Ser. No. 13/639,927, contents of which are incorporated herein in their entirety.

In some embodiments, immune cells or precursors thereof of the present invention include CD4⁺ cells. In some embodiments, immune cells or precursors thereof of the present invention include CD25⁺ cells. In some embodiments, immune cells or precursors thereof of the present invention include CD25^{high} cells. In some embodiments, immune cells or precursors thereof of the present invention include CD127⁻ cells. In some embodiments, immune cells or precursors thereof of the present invention include CD127^{low} cells. In some embodiments, immune cells or precursors thereof of the present invention include CD45RA⁺ cells. In some embodiments, immune cells or precursors thereof of the present invention include CD4⁺, CD25^{high}, CD127^{low}, and/or CD45RA⁺ cells.

### Expansion of Immune Cells

Whether prior to or after modification of cells to express a subject CAR, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005. For example, the immune cells of the invention may be expanded by contact with a surface having attached thereto an agent that stimulates a CD3/TCR complex associated signal and a ligand that stimulates a co-stimulatory molecule on the surface of the immune cells. In particular, immune cell populations may be stimulated by contact with an anti-CD3 antibody, or an antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the immune cells, a ligand that binds the accessory molecule is used. For example, immune cells can be contacted with an anti-CD3 antibody and an anti-CD28 antibody, under conditions appropriate for stimulating proliferation of the immune cells. Examples of an anti-CD28 antibody include 9.3, B-T3, XR-CD28 (Diaclone, Besancon, France) and these can be used in the invention, as can other methods and reagents known in the art (see, e.g., ten Berge et al., Transplant Proc. (1998) 30(8): 3975-3977; Haanen et al., J. Exp. Med. (1999) 190(9): 1319-1328; and Garland et al., J. Immunol. Methods (1999) 227(1-2): 53-63).

Expanding the immune cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the immune cells expand in the range of about 20 fold to about 50 fold.

Following culturing, the immune cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The immune cell medium may be replaced during the culture of the immune cells at any time. Preferably, the immune cell medium is replaced about every 2 to 3 days. The immune cells are then harvested from the culture apparatus whereupon the immune cells can be used immediately or cryopreserved to be stored for use at a later time. In one embodiment, the invention includes cryopreserving the expanded immune cells. The cryopreserved immune cells are thawed prior to introducing nucleic acids into the immune cell.

In another embodiment, the method comprises isolating immune cells and expanding the immune cells. In another embodiment, the invention further comprises cryopreserving the immune cells prior to expansion. In yet another embodiment, the cryopreserved immune cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of immune cells comprises the addition to the cellular growth factors, such as those described in U.S. Pat. No. 5,199,942, or other factors, such as flt3-L, IL-1, IL-3 and c-kit ligand. In one embodiment, expanding the immune cells comprises culturing the immune cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for immune cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α. or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of immune cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

The medium used to culture the immune cells may include an agent that can co-stimulate the immune cells. For example, an agent that can stimulate CD3 is an antibody to CD3, and an agent that can stimulate CD28 is an antibody to CD28. This is because, as demonstrated by the data disclosed herein, a cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the immune cells expand in the range of about 20 fold to about 50 fold, or more by culturing the electroporated population. In one embodiment, human T regulatory cells are expanded via anti-CD3 antibody coated KT64.86 artificial antigen presenting cells (aAPCs). Methods for expanding and activating immune cells can be found in U.S. Pat. Nos. 7,754,482, 8,722,400, and 9,555,105, contents of which are incorporated herein in their entirety.

In one embodiment, the method of expanding the immune cells can further comprise isolating the expanded immune cells for further applications. In another embodiment, the method of expanding can further comprise a subsequent electroporation of the expanded immune cells followed by culturing. The subsequent electroporation may include introducing a nucleic acid encoding an agent, such as a transducing the expanded immune cells, transfecting the expanded immune cells, or electroporating the expanded immune cells with a nucleic acid, into the expanded population of immune cells, wherein the agent further stimulates the immune cell. The agent may stimulate the immune cells, such as by stimulating further expansion, effector function, or another immune cell function.

### Methods of Treatment

The modified immune cells (e.g., regulatory T cells) described herein may be included in a composition for immunotherapy, in particular suppression immunotherapy. The composition may include a pharmaceutical composition and further include a pharmaceutically acceptable carrier. A therapeutically effective amount of the pharmaceutical composition comprising the modified immune cells may be administered.

In one aspect, the invention includes a method for adoptive cell transfer therapy comprising administering to a subject in need thereof a modified immune cell (e.g., regulatory T cell) of the present invention. In another aspect, the invention includes a method of treating a disease or a condition in a subject comprising administering to a subject in need thereof a population of modified immune cells.

Generally, the method of treatment comprises several steps prior to the generation of modified immune cells suitable for therapy. The steps may include: (1) obtaining a blood sample from a subject; (2) leukapheresis of the blood sample to enrich for white blood cells; and (3) FACS-based isolation of immune cells, e.g., based on cell surface markers. Following the isolation of immune cells, viral transduction of the immune cells to express a subject CAR is performed, and expansion of the transduced cells is induced. Methods of expansion are described elsewhere herein, and may include pan-stimulation with artificial antigen-presenting cells, and contacting the transduced immune cells with cytokines (e.g., IL-2). Washing and concentration steps may be performed on the expanded population of CAR-expressing immune cells thereby generating the pharmaceutical composition. The pharmaceutical composition is then administered into a subject in need thereof at a therapeutically effective amount.

In some embodiments, the method of treatment comprises several steps prior to the generation of modified regulatory T cells suitable for therapy. The steps may include: (1) obtaining a blood sample from a subject; (2) leukapheresis of the blood sample to enrich for white blood cells; and (3) FACS-based isolation of regulatory T cells, e.g., based on cell surface markers, e.g., CD4⁺, CD25^{high}, CD127^{low}, and/or CD45RA⁺. Following the isolation of regulatory T cells, viral transduction of the regulatory T cells to express a DPP6 or FAP CAR is performed, and expansion of the transduced cells is induced. Methods of expansion are described elsewhere herein, and may include pan-stimulation with artificial antigen-presenting cells, and contacting the transduced regulatory T cells with cytokines (e.g., IL-2). Washing and concentration steps may be performed on the expanded population of DPP6 or FAP specific CAR-Treg cells thereby generating the pharmaceutical composition. The pharmaceutical composition is then administered into a subject in need thereof at a therapeutically effective amount.

In one embodiment, the method of treating a disease or condition in a subject in need thereof comprises administering to the subject a therapeutically effective amount of a modified cell (e.g. Treg) comprising a subject CAR (e.g., DPP6 or FAP CAR). In one embodiment, the method of treating a disease or condition in a subject in need thereof comprises administering to the subject a therapeutically effect amount of a modified cell (e.g. Treg) comprising a subject CAR (e.g., DPP6 or FAP CAR), wherein the subject CAR comprises an antigen binding domain that can bind to DPP6 or FAP. In one embodiment, the DPP6 specific CAR comprises a CD8 signal peptide, an DPP6 Vim domain, a CD8 hinge or IgG4 spacer region, a CD28 transmembrane domain, a CD28 costimulatory domain, and a CD3ζ intracellular domain. In another embodiment, the FAP specific CAR comprises an FAP binding domain, a CD8 hinge or IgG4 spacer region, a CD8 transmembrane domain, a CD28 costimulatory domain, and a CD3ζ intracellular domain.

The DPP6 or FAP CAR of the invention is able to redirect immune cells (e.g., regulatory T cells) to targets expressing the DPP6 or FAP antigen, especially β-islet cells of the pancreas. As such, the subject CAR of the invention is an antigen-specific CAR. Tregs expressing an DPP6 or FAP CAR of the invention upon activation by DPP6 or FAP binding, induces proliferation of the modified Tregs and enhances the suppressor function of the modified Tregs.

When a modified immune cell comprising a subject CAR of the invention is administered, the β-islet cells of the pancreas are protected from autoimmune destruction. In one embodiment, a modified immune cell comprising a subject CAR of the invention (e.g., a Treg comprising an DPP6 or FAP CAR) can mediate DPP6-specific or FAP-specific immunosuppression. In one embodiment, a modified immune cell comprising a subject CAR of the invention (e.g., a Treg comprising an DPP6 or FAP CAR) can suppress autoimmune T cell proliferation in response to self-tissues (e.g., tissues expressing DPP6 or FAP antigen). In such cases, substantial immune cell infiltration into the affected tissues, and/or organs may occur, resulting in destruction of the cells, tissues, and/or organs. Accordingly, in some embodiments, a modified immune cell comprising a subject CAR of the invention (e.g., a Treg comprising an DPP6 or FAP CAR), is capable of reducing infiltration of autoimmune cells, and thus protecting the normal cells, tissues, and/or organs from destruction. Accordingly, in some embodiments, a modified immune cell comprising a subject CAR of the invention (e.g., a Treg comprising an DPP6 or FAP CAR), is able to reduce autoimmune mediated toxicity and diseases or disorders resulting therefrom.

Accordingly, the present invention provides a method for achieving a preventative therapeutic effect in a subject in need thereof, and/or a method for achieving an immunosuppressive effect in a subject in need thereof e.g. one who is experiencing and/or suffering from an autoimmune response. In some embodiments, a method for achieving a preventative therapeutic effect in a subject in need thereof, and/or a method for achieving an immunosuppressive effect in a subject in need thereof with an autoimmune response, comprises administering to the subject a modified immune cell comprising a subject CAR of the invention. In one embodiment, the present invention provides a method for achieving an immunosuppressive effect in a subject in need thereof with an autoimmune response, comprising administering to the subject a modified regulatory T cell comprising a chimeric antigen receptor (CAR) having affinity for DPP6, wherein the CAR comprises an DPP6 binding domain, a CD8 hinge domain or IgG4 spacer domain, a CD28 transmembrane domain, a CD28 costimulatory domain, and a CD3ζ intracellular domain. In one embodiment, the present invention provides a method for achieving an immunosuppressive effect in a subject in need thereof with an autoimmune response, comprising administering to the subject a modified regulatory T cell comprising a chimeric antigen receptor (CAR) having affinity for FAP, wherein the CAR comprises an FAP binding domain, a CD8 hinge domain or IgG4 spacer domain, a CD28 transmembrane domain, a CD28 costimulatory domain, and a CD3ζ intracellular domain. In one embodiment, the present invention provides a method for achieving a preventative therapeutic effect in a subject in need thereof, comprising administering to the subject a modified regulatory T cell comprising a chimeric antigen receptor (CAR) having affinity for DPP6 or FAP, wherein the CAR comprises an DPP6 or FAP binding domain, a CD8 hinge domain or IgG4 spacer domain, a CD28 transmembrane domain, a CD28 costimulatory domain, and a CD3ζ intracellular domain.

Type 1 diabetes is a T cell-mediated autoimmune disease resulting in islet beta-cell destruction, hypoinsulinemia, and severely altered glucose homeostasis. Failure of regulatory T cells (Tregs) may play a role in the development of type 1 diabetes. During immune homeostasis, Tregs counterbalance the actions of autoreactive effector T cells, thereby participating in peripheral tolerance. Thus, an imbalance between effector T cells and Tregs may contribute to the breakdown of peripheral tolerance, leading to the development of type 1 diabetes. In some embodiments, a modified immune cell comprising a subject CAR of the invention (e.g., a Treg comprising an DPP6 or FAP CAR), is capable of suppressing T cell-mediated autoimmune diseases, such as type 1 diabetes. Accordingly, the present invention provides a method of treating diabetes in a subject in need thereof, comprising administering to the subject a modified immune cell comprising a subject CAR of the invention. In some embodiments, a method of treating diabetes in a subject in need thereof is provided, comprising administering to the subject a modified regulatory T cell comprising a chimeric antigen receptor (CAR) having affinity for DPP6 or FAP, wherein the CAR comprises an DPP6 or FAP binding domain, a CD8 hinge domain or IgG4 spacer domain, a CD28 transmembrane domain, a CD28 costimulatory domain, and a CD3ζ intracellular domain. In some embodiments, the diabetes is type I diabetes.

In certain embodiments, the CAR is encoded by the nucleic acid sequence of SEQ ID Nos: 16, 25, 32, 39, and/or 63. In certain embodiments, the CAR comprises the amino acid sequence of SEQ ID NOs: 15, 24, 31, and/or 38.

In certain embodiments, the modified immune cell is a modified regulatory T cell (Treg). In some embodiments, the modified immune cell is an autologous cell. In some embodiments, the modified immune cell (e.g., modified regulatory T cell) is derived from a human.

The CAR can redirect the T regulatory cell to DPP6 or FAP expressing tissue, thus enhancing protection of the DPP6 or FAP expressing tissue from autoimmune destruction.

### Non-Human Primate Models of Type 1 Diabetes

Spontaneous, immune-mediated type 1 diabetes is relatively rare in non-human primate species as compared to human populations, which has made development of experimental models of T1D in non-human primates challenging. Existing non-human primate models of T1D typically involve the depletion of islet cells through non-immune methods such as surgical resection of the pancreas and/or the use of selectively toxic drugs such as streptozotocin (STZ), which is particularly toxic to insulin-producing cells such as beta islet cells. Often surgical resection is combined with STZ treatment. While these models result in the development of insulin-dependent diabetes mellitus, they do not involve an autoimmune-based mechanism, and thus cannot be used to study potential treatments or biological phenomena related to T cell- or auto-immune-mediated depletion of beta islet cells as seen in human cases of T1D. As such, in another aspect, the invention includes a non-human primate model of type 1 diabetes, comprising administering to a non-human primate subject an effective amount of a modified T cell comprising a chimeric antigen receptor (CAR) having an affinity for a islet cell antigen. In this way, effector CAR T cells are generated which target and kill islet cells, thereby recapitulating both the causal mechanism and clinical outcome of T1D. In certain embodiments, the CAR has an affinity for DPP6. In certain embodiments, the CAR has an affinity for fibroblast activation protein (FAP). It is also contemplated that CAR T cells specific for DPP6 and CAR T cells specific for FAP could be used in combination in order to optimize the desired auto-immune effect.

In certain embodiments, the administration of islet-targeting CAR T cells can be combined with other treatments in order to more closely model biological processes which take place in human T1D patients. As a non-limiting example, in certain embodiments, the non-human primate model further comprises the administration of streptozotocin, wherein the amount of streptozotocin is sufficient to induce islet cell injury but not depletion. The STZ administration can happen either prior to, concurrent with, or following administration of the modified T cells. The skilled artisan would be able to select a timing of modified T cell and STZ administration depending the scientific needs of the study being performed. In certain embodiments, the non-human primate model further comprises the administration of an effective amount of an immune-modulating agent. In certain embodiments, the immune-modulating agent is a CRISPR-based system which modified the expression of one or more immune-associated genes either by mutation of germline DNA or the depletion of mRNA. In certain embodiments, the CRISPR-based system disrupts the expression of an immune checkpoint protein. Several immune checkpoint proteins and genes are known in the art to play key roles in regulating immune responses, including auto-immune responses, including but not limited to PD-1, CTLA-4, TIM3, GITR, BTLA, LAG3, among others.

A number of non-human primate species have been used for diabetes mellitus animal models including, but not limited to cynomolgus macaques, rhesus macaques, baboons, and chimpanzees. Of these species, macaque monkeys have been a common choice for a number of reasons including relative easy handling and upkeep in captivity, commercial availability, and similarities to humans in major histocompatibility complex (MHC) genes polymorphism, immune function, and overall physiology.

Any number of routes of administration can be used to administer the islet targeting modified T cells in the model of the invention. In certain embodiments, the modified T cells are administered via the splenic artery so that they have more direct access to pancreatic tissue. In certain embodiments, the route of administration is one of the methods described in the “Pharmaceutical compositions” section of the present disclosure.

### Pharmaceutical Compositions

Pharmaceutical compositions of the present invention may comprise the modified immune cell as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

The cells of the invention to be administered may be autologous, allogeneic or xenogeneic with respect to the subject undergoing therapy.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

Also provided are populations of immune cells of the invention, compositions containing such cells and/or enriched for such cells, such as in which cells expressing the recombinant receptor make up at least 50%, 60%, 70%, 80%, 90%, 91%, 92%, 93%, 94%, 95%, 96%, 97%, 98%, 99%, or more of the total cells in the composition or cells of a certain type such as regulatory T cells. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980).

Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

It can generally be stated that a pharmaceutical composition comprising the modified immune cells described herein may be administered at a dosage of 10⁴ to 10⁹ cells/kg body weight, in some instances 10⁵ to 10⁶ cells/kg body weight, including all integer values within those ranges. Immune cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

The administration of the modified immune cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Example 1: Construction of an Anti-DPP6 Chimeric Antigen Receptor

DPP6 is a transmembrane protein that associates with Kv4 family voltage gated potassium channels to regulate their function. A recent publication has described the use of RNA sequencing and qRTPCR of human pancreatic islet to identify DPP6 as a preferentially expressed transcript in alpha and beta pancreatic islet cells (Balhuizen, et al. (2017) Science Reports. 7(1):15130). DPP6 was also found to be expressed in the brain, colon, testes, and thyroid. The authors then immunized a dromedary camel with recombinant extracellular DPP6 protein in order to generate camelid nanobody molecules with binding specificity for DPP6. Purification of the resulting nanobodies was then performed via selection with phage-ELISA. The soluble forms of four resulting nanobodies (4hD29, 2hD1, 2hD6, and 2hD38) were found to be able to bind to DPP6+Kelly neuroblastoma cells. While developed for histology and diagnostic applications, these anti-DPP6 nanobodies can also be adapted for use as antigen-binding domains of chimeric antigen receptor (CAR) constructs. Expression of the DPP6 CARs in CD4+ regulatory T cells (Treg) would enable the use of these cells to prevent or ameliorate the autoimmune responses responsible for β-islet cell depletion in type I diabetes or after islet cell transplantation. Previous studies have shown that polyclonal Tregs can delay progression of type I diabetes and that these cells persist for up to a year in transfused patients. Without wishing to be bound by theory, introducing CARs into Treg cells is an attractive way to generate antigen-specific Treg cells. In addition to reducing the number of Treg cells that are required for an effective response, antigen specificity should restrict the trafficking and off-target suppression of injected Treg cells. Further, the expression of DPP6 CARs in cytotoxic T cells, with appropriate safety mechanisms, would have uses in treating insulinomas, pancreatic cancer, or congenital hyperinsulinsim whereby DPP6 CAR Teffs could eliminate pathogenic islet cells.

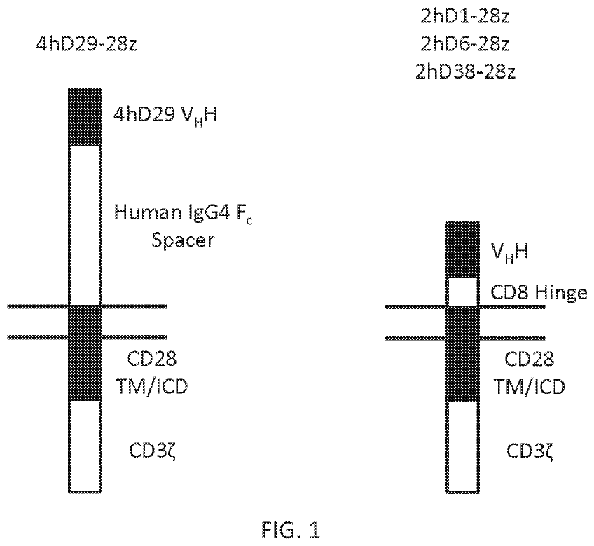

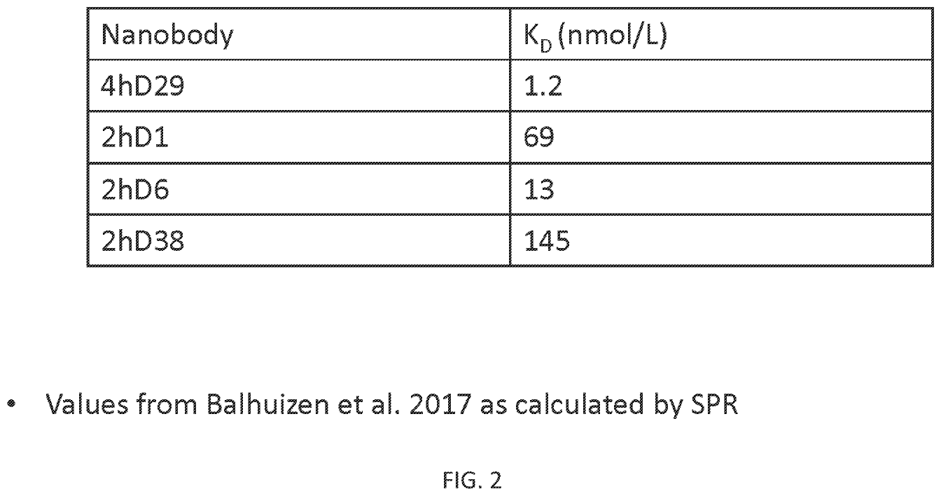

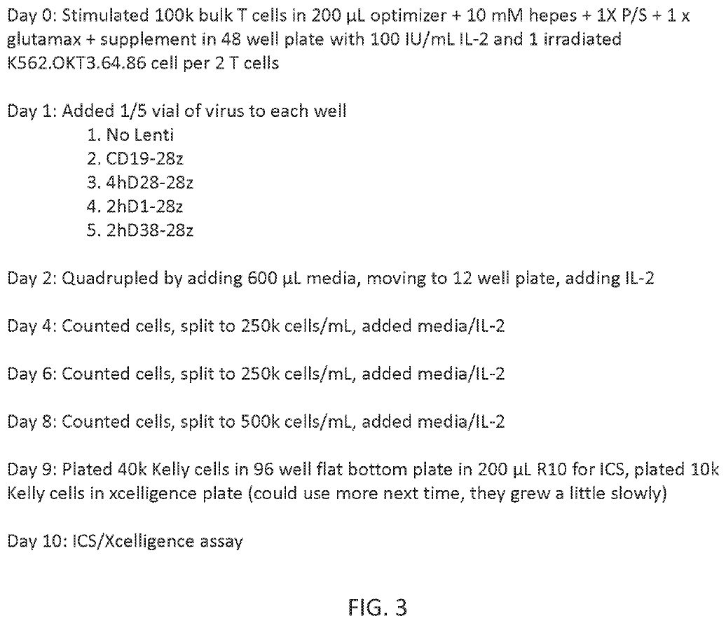

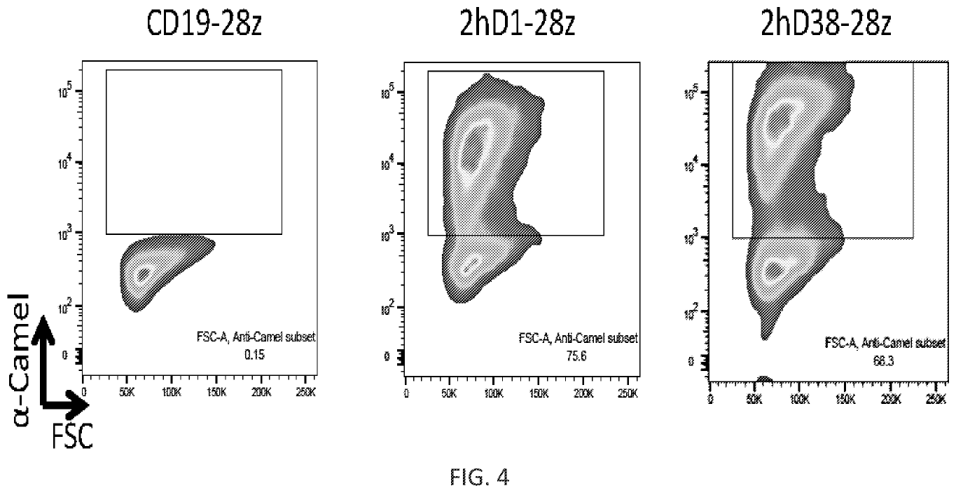

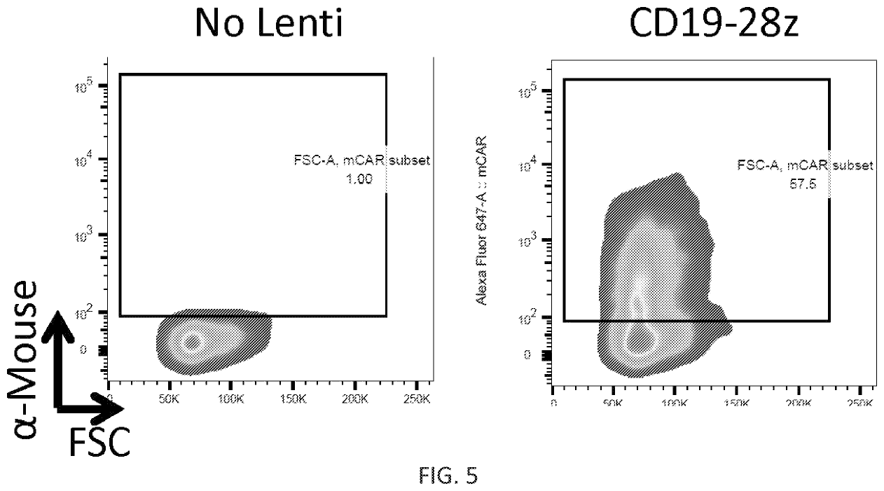

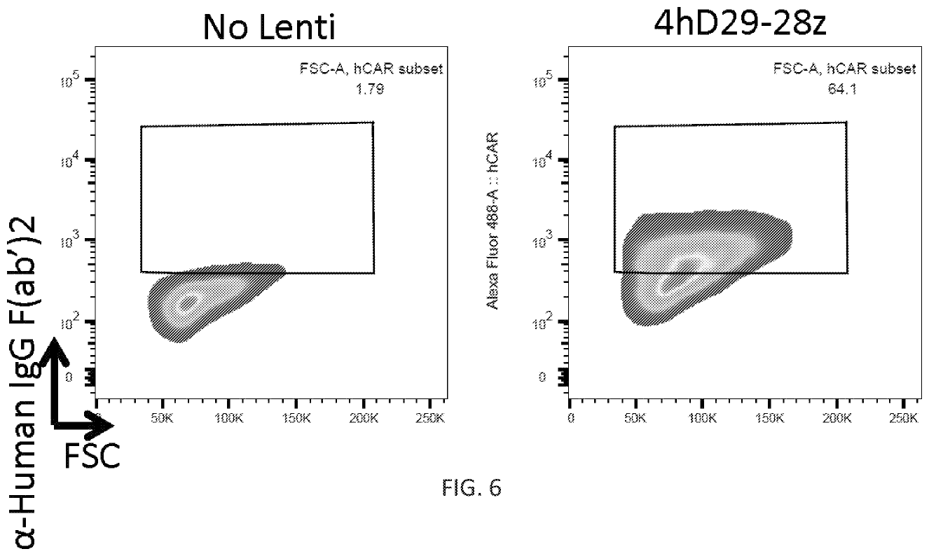

Fusion proteins were created comprising the VHH domains from 4hD29, 2hD1, 2hD6, and 2hD38 nanobodies, either a CD8 hinge or a human IgG4 Fc spacer, CD28 transmembrane and intracellular domains, and a CD3 signaling domain (FIG. 1). The 4hD29-28z construct contained the IgG4 spacer, while the 2hD1-28z, 2hD6-28z, and 2hD38-28z constructs included CD8 hinge domains. FIG. 2 shows the affinities of these four nanobody domains for DPP6 protein, as calculated by surface plasmon resonance, which range from 1.2 nmol/L for 4hD29 to 145 nmol/L for 2hD38. The constructs were then cloned into expression vectors and packaged into lentiviral particles to enable their transfer and expression in human T cells. FIG. 3 describes the workflow for the transduction, expansion, and verification of T cells with the four DPP6 CARs. As controls, one group was transduced with a CD19-28z CAR construct, while another group received no transduction. Flow cytometry staining was used to verify expression of the CAR constructs using either anti-camel or anti-mouse (for the CD19-28z CAR) primary antibodies (FIG. 4 and FIG. 5). An anti-human IgG primary antibody was used to stain T cells expressing the 4hD29-28z construct (FIG. 6). These data demonstrated the successful transduction of T cells and robust expression of the CAR constructs.

### Example 2: Functional Evaluation of DPP6 CARs

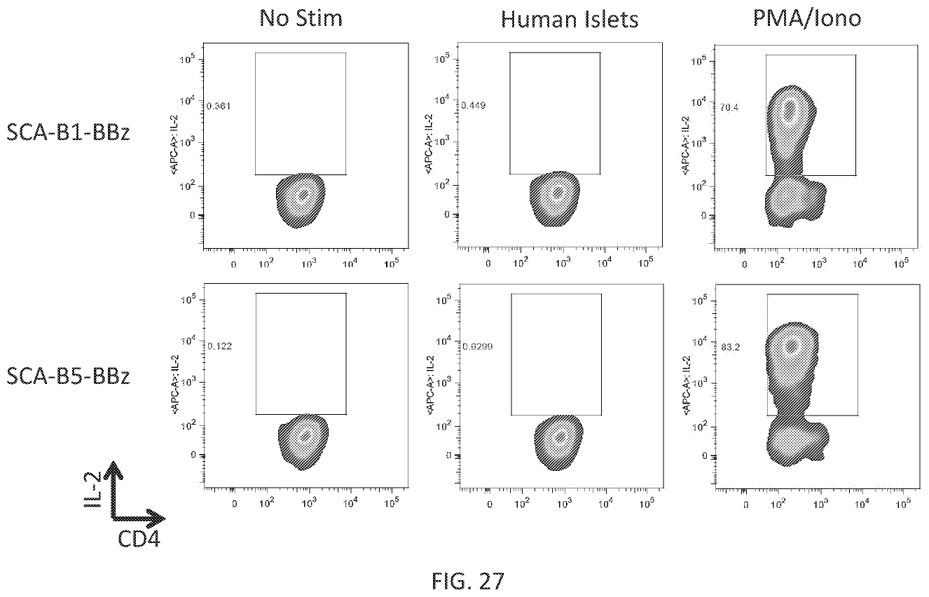

Studies were then conducted to determine whether the DPP6 CAR constructs were capable of activating T cells upon binding of their antigen expressed by target cells. These studies were a key part of development, as not every antibody or nanobody adapted into a CAR molecule results in a functioning construct. FIG. 27 is an example of two CARs created from antibodies specific for two other islet cell proteins: SCA-B1 and SCA-B5. Despite published reports of the ability of anti-SCA-B1 and anti-SCA-BS antibodies to stain human islets in immunofluorescence assays, CARs created from these antibodies did not display antigen-specific cytokine production when incubated with human islet cells. These studies demonstrated that imaging antibodies cannot always be successfully converted for use in CAR constructs, which highlights the novel benefiti of the anti-DPP6 nanobody-based CARs of the invention.

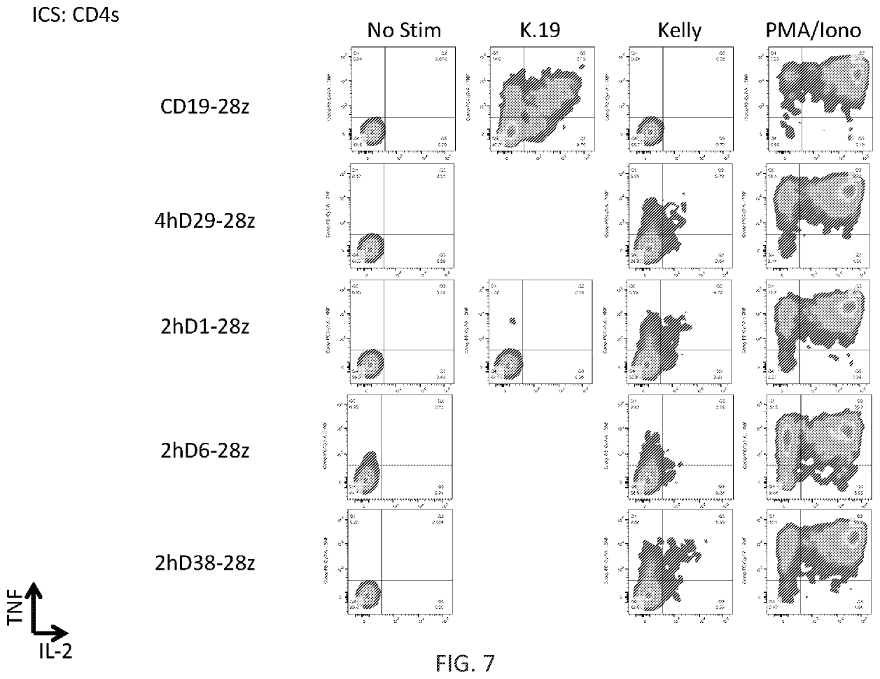

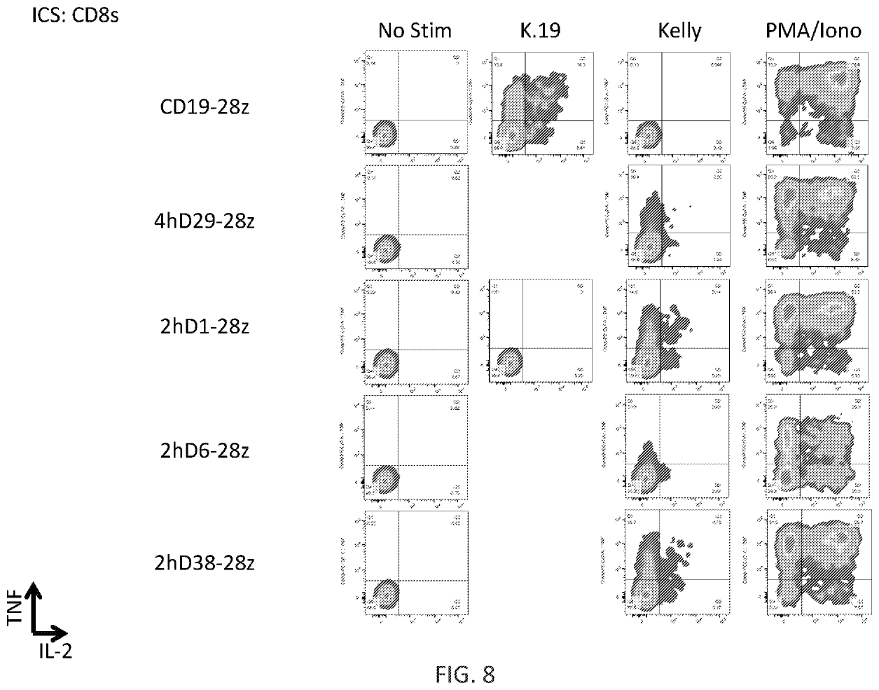

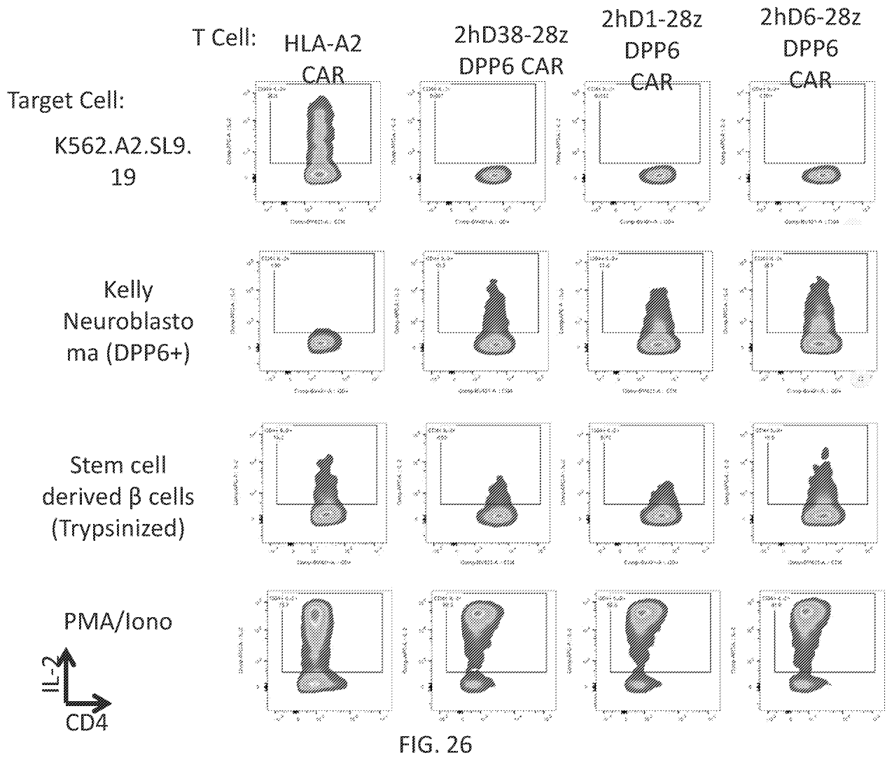

At the end of the 10-day expansion culture, CAR function was verified using intracellular cytokine staining (ICS) or Xcelligence assay using DPP6-expressing Kelly neuroblastoma cells as targets. Stimulation of transduced cells with antigen-expressing target cells (Kelly neuroblastoma or non-DPP6 expressing K.19 cells) or PMA/ionomycin resulted in TNF and IL-2 production by both transduced CD4+ and CD8+ T cells (FIG. 7 and FIG. 8). To setup the stimulation assays, 100 k T cells were plated in 200 μL final volume of R10 media, followed by adding 200 k Kelly or K562.A2.SL9.19 to the wells. Golgiplug was then added and the plate was incubated for 4 hours, followed by permeabilization and staining for cytokines. 2hD6-28z cells used in these studies were previously cryopreserved following expansion. A similar study was also performed using stem cell derived β-cells as targets (FIG. 26).

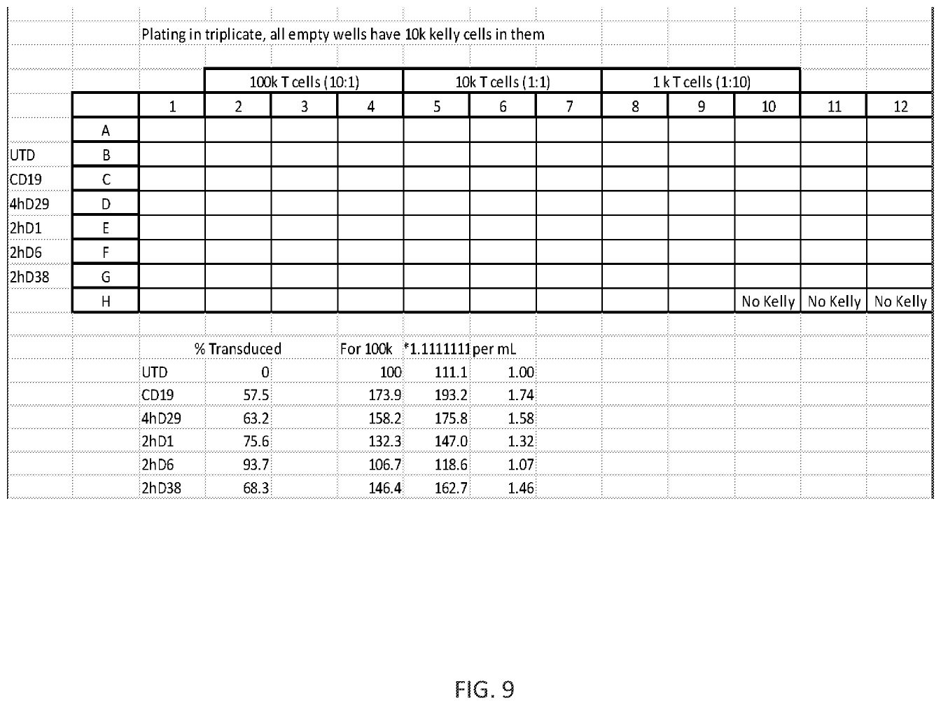

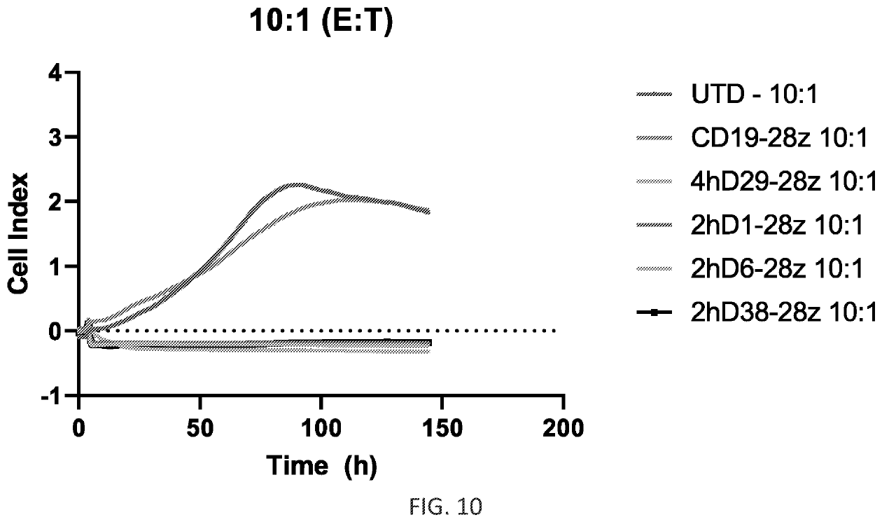

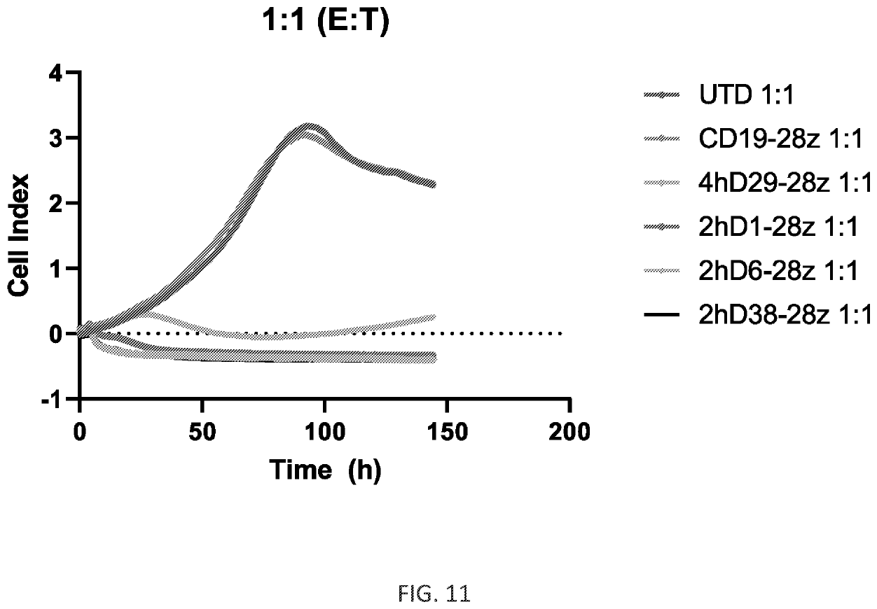

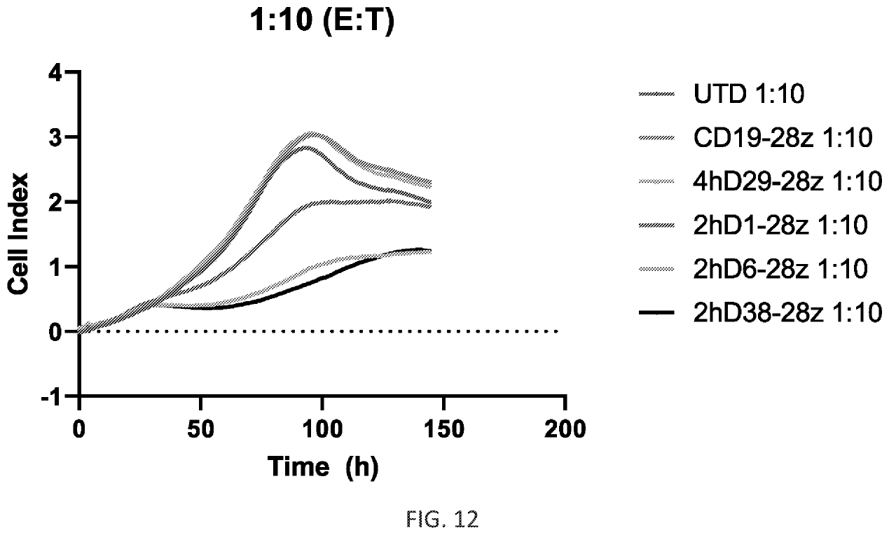

Having demonstrated the ability to produce cytokine after encountering antigen, DPP6 CAR expressing T cells were then evaluated for the ability to kill target cells. In these studies, Xcelligence real-time quantitative cell analysis was used to monitor target cell growth (setup and conditions described in FIG. 9). The ability of CAR-expressing T cells to kill target cells over a ˜150 hour study at various effector to target ratios is depicted in FIGS. 10, 11, and 12. As expected, untransduced T cells and T cells expressing the CD19-28z were unable to control the growth of target cells, while all four DPP6 CARs killed target cells. Of note, the 4hD29-28z and 2hD38-28z CARs were best able to suppress target cells at an effector to target ratio of 1:10.

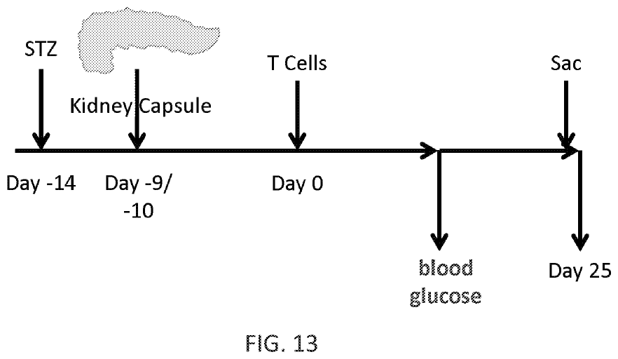

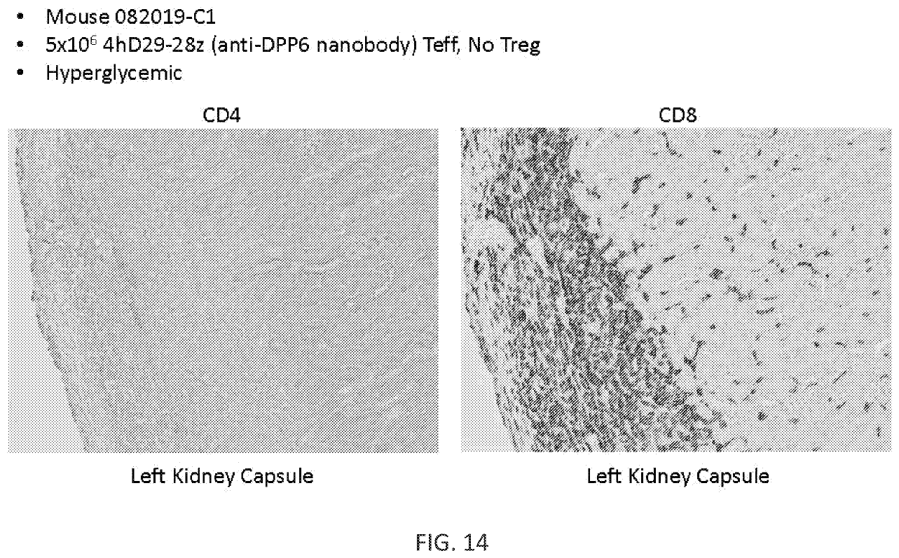

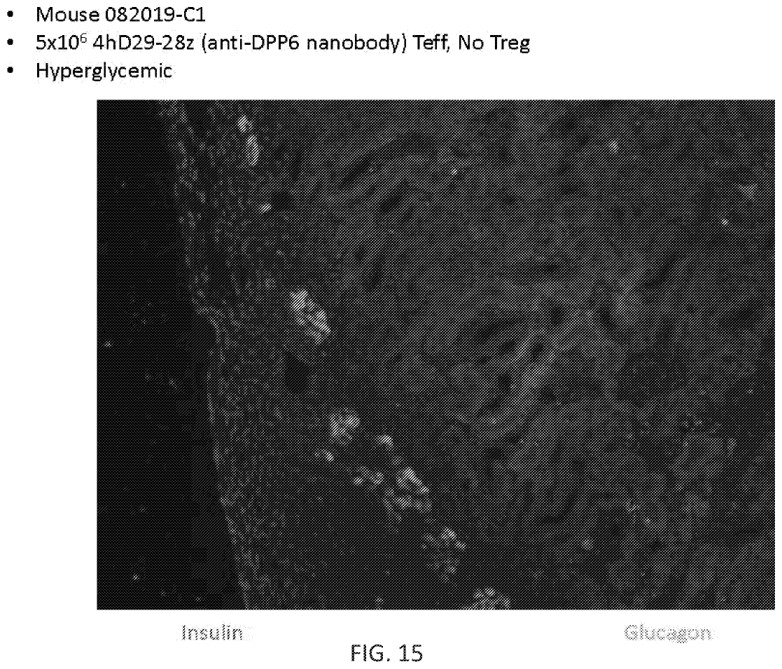

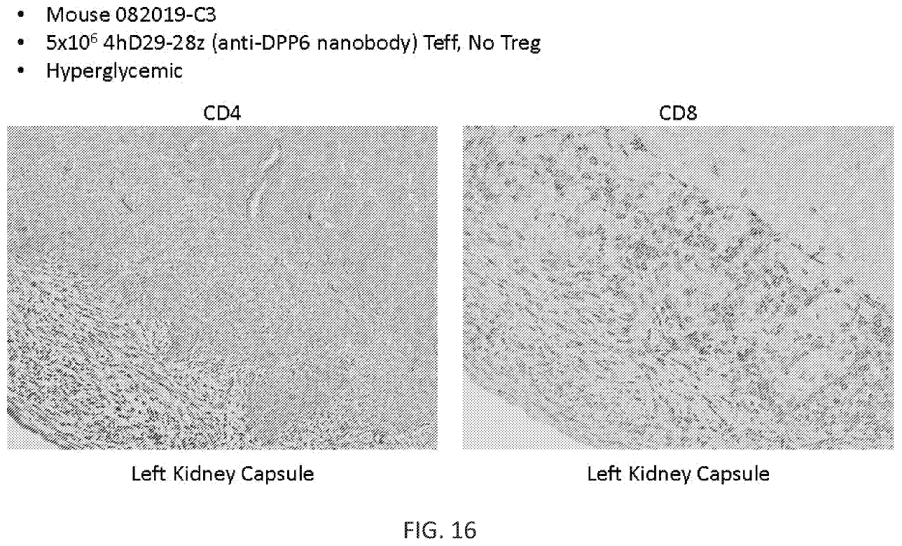

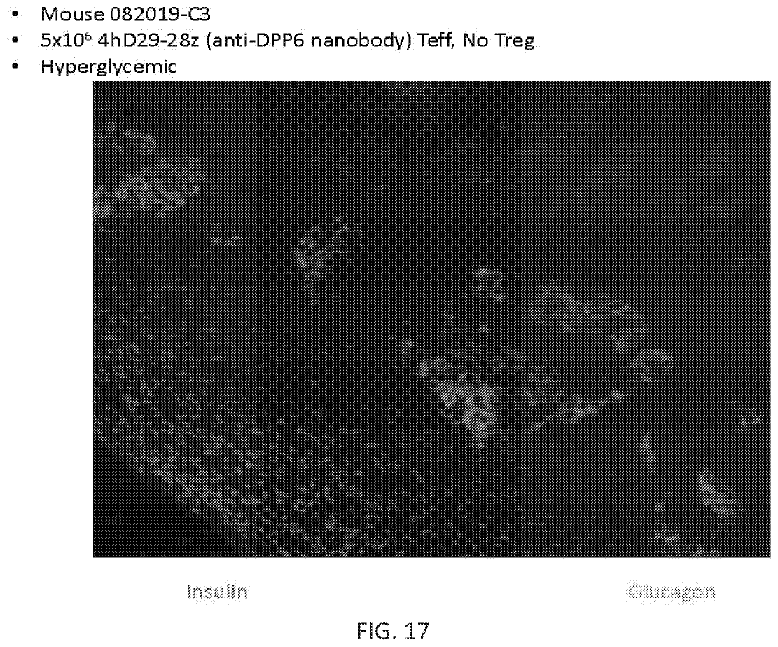

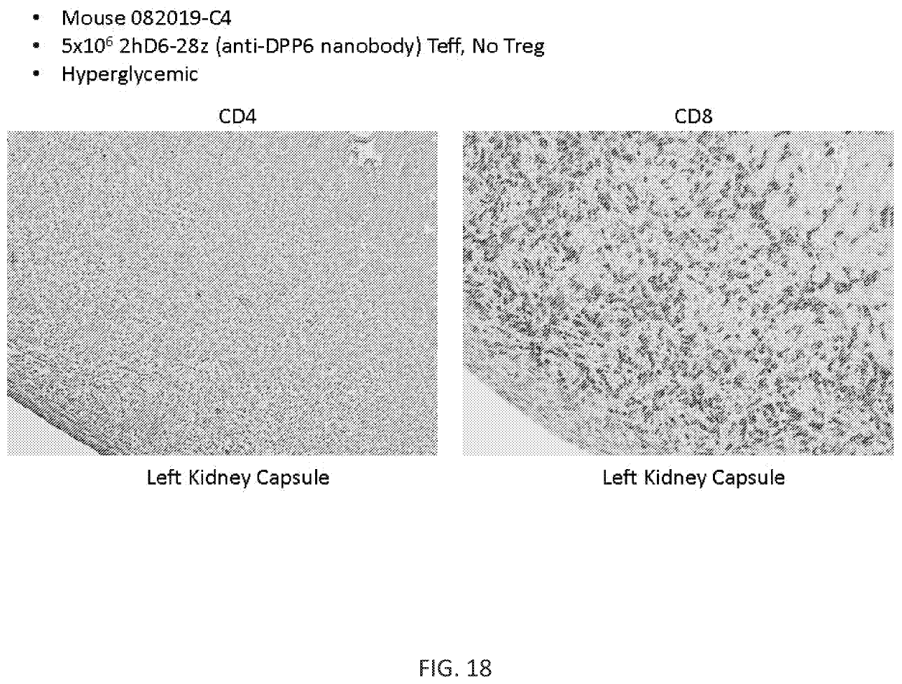

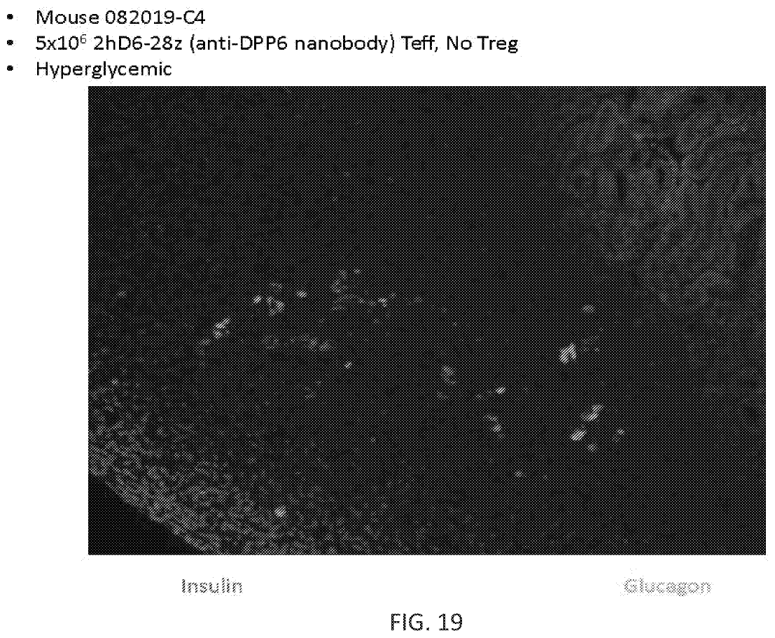

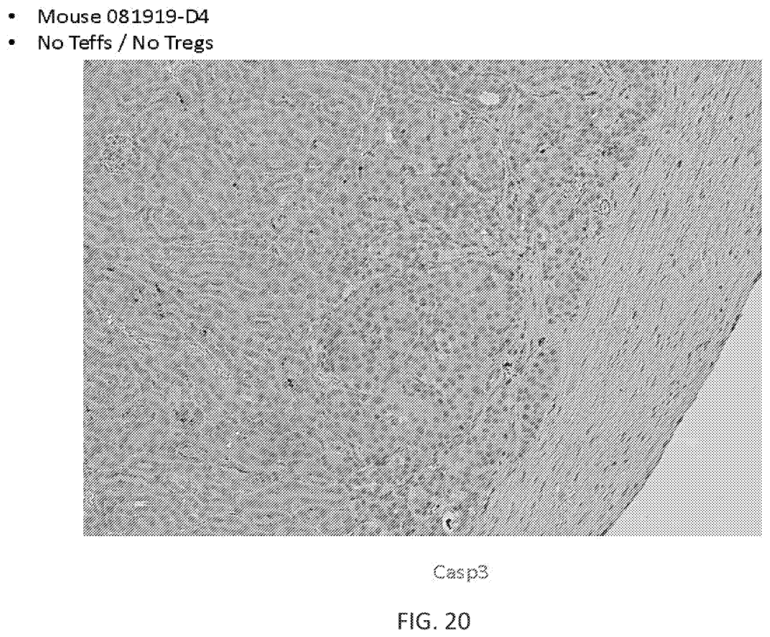

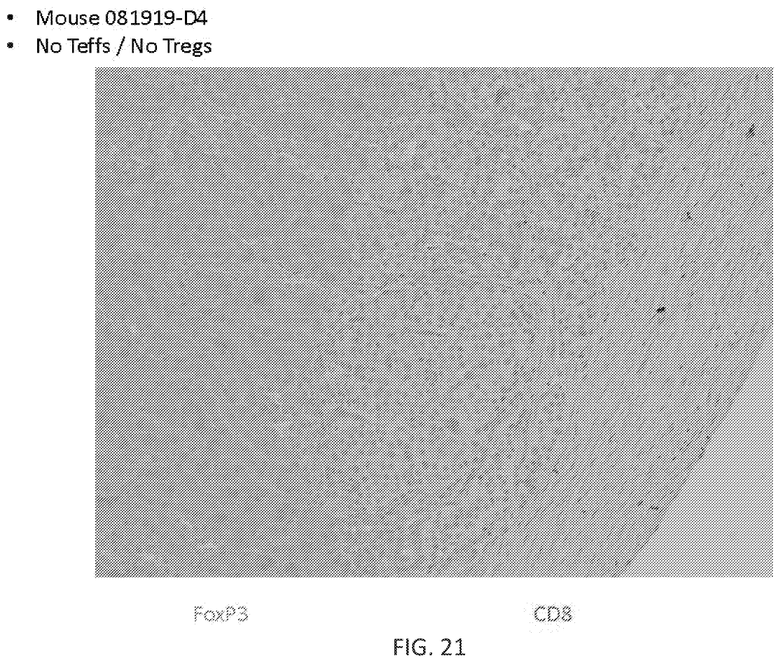

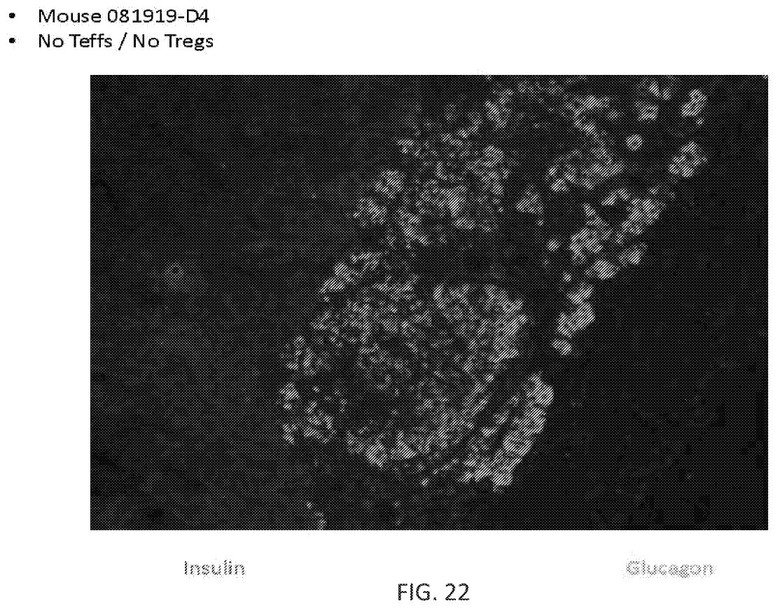

The functional ability of DPP6 CARs in vivo was then evaluated using NSG mice transplanted with human islet cells after reduction of endogenous islet cells by streptozotocin treatment (FIG. 13). Following injection of DPP6 CAR expressing T cells, blood glucose was monitored followed by harvest of kidney tissue at day 25 for hisological analysis (transplanted islet cells were placed under the kidney capsule). Mice injected with 4hD29-28z and 2hD6-28z expressing T cells demonstrated hyperglycemia and histological staining demonstrated both significant T cell infiltrate in the kidney capsule (FIGS. 14, 16, and 18) and loss of insulin-expressing cells (FIGS. 15, 17, and 19). In contrast, mice receiving no T cells demonstrated little to no cell death marker expression (FIG. 20) or insulin-expressing cell loss (FIG. 22), which was not due to the presence of regulatory T cells (FIG. 21). In total, these data indicated the ability of DPP6 CARs to recognize and kill human islet cells in vivo.

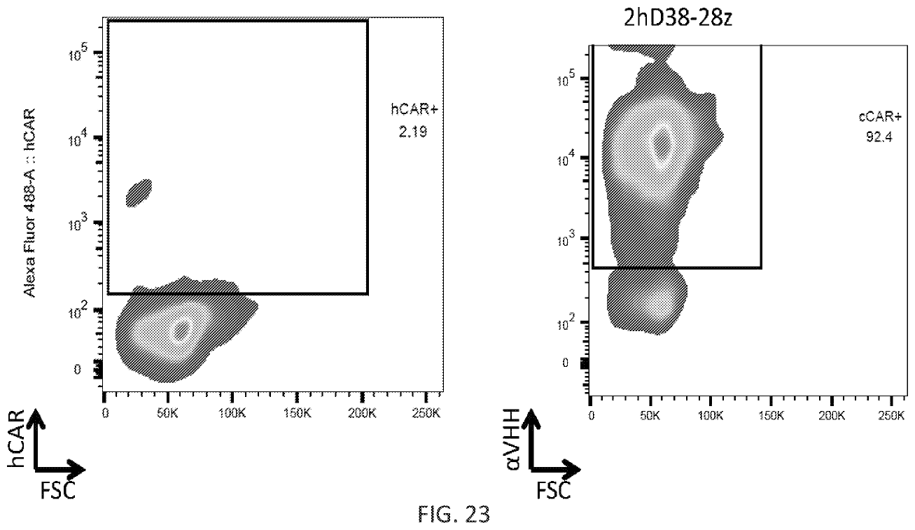

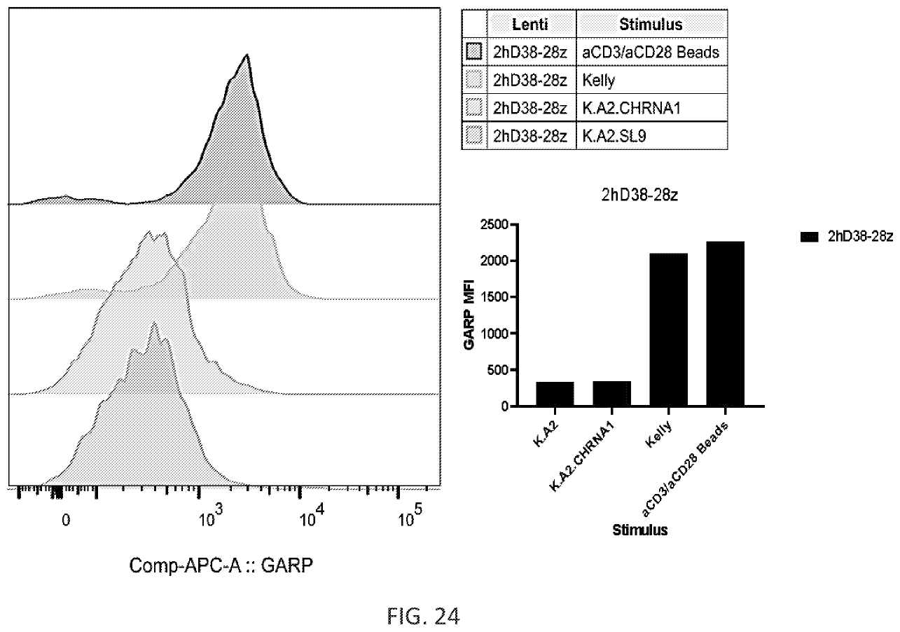

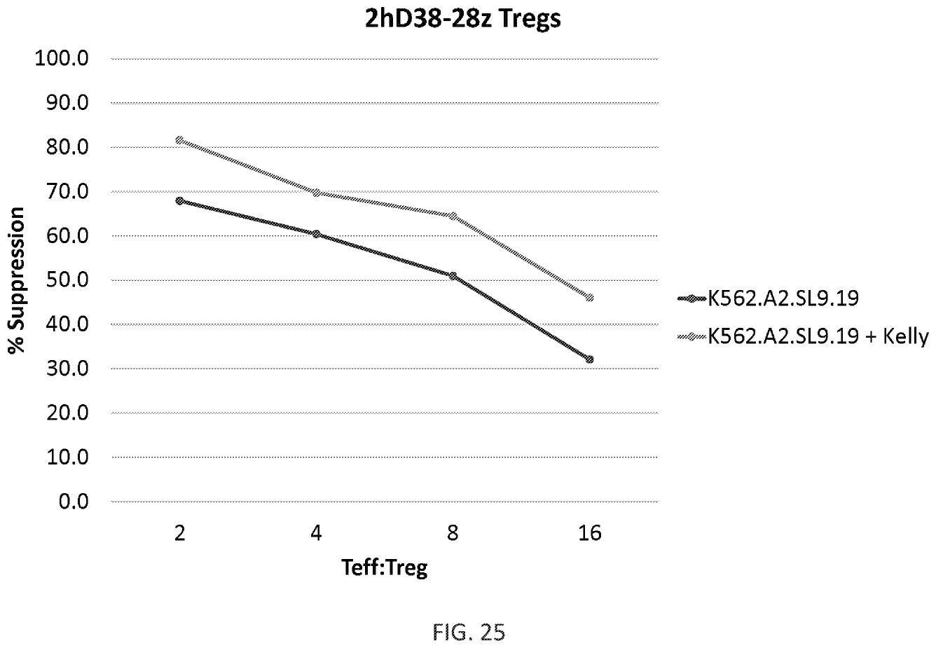

Lastly, the ability of DPP6 CAR constructs to activate CD4+ regulatory T cells (Treg) was then determined. Human Treg were sorted, stimulated, and transduced with 2hD38-28z. Following expansion, expression of the CAR was first verified by flow cytometry (FIG. 23). CAR expressing Tregs were then activated with DPP6+ Kelly neuroblastoma cells or αCD 3/αCD28 followed by extracellular staining for GARP expression, a Treg specific marker of activation (FIG. 24). Lastly, DPP6 CAR expressing Tregs were used in in vitro suppression assays to verify antigen-specific function. Expanded 2hD38-28z Tregs were incubated in either uncoated wells (K562.A2.SL9.19) or wells coated with irradiated Kelly cells (K562.A2.SL9.19+Kelly). After 4 days, CFSE dilution among bystander CD8 T cells was measured. (FIG. 25). Results from this study clearly demonstrated the suppressive ability of CAR-expressing Treg. Without wishing to be bound by theory, these results indicated the potential for DPP6 CAR expressing Tregs to suppress autoimmune β-islet cell specific T cells in a clinical setting in order to manage or reverse type 1 diabetes.

### Example 3: Generation of a Non-Human Primate Model (NHP) of Type I Diabetes (T1D)

Immune-based non-human primate (NHP) models of type I diabetes do not exist, limiting the ability to do careful preclinical studies prior to the initiation of Phase I human clinical trials aimed to test and improve upon promising therapies to prevent, or cure T1D. Non-human primates spontaneously acquire T1D but do so at a substantially lower rate than what is observed in the human population, making natural cohort therapeutic studies near impossible to perform. Similarly, existing NHP models of T1D using rhesus or cynomolgus macaques require the depletion of islet cells through non-immune methods such as surgery or streptozotocin (STZ) treatment. Nonetheless, those primates who do acquire T1D manifest a disease course that is similar to that observed in humans, suggesting that studying T1D in NHP models will provide valuable insights into how to treat T1D in humans.

The studies of the current disclosure focus on generating a chimeric antigen receptor (CAR)-based model of inducing T1D in NHPs that is useful to study T1D disease initiation, pathology and therapeutic approaches to prevent, delay or reverse T1D.

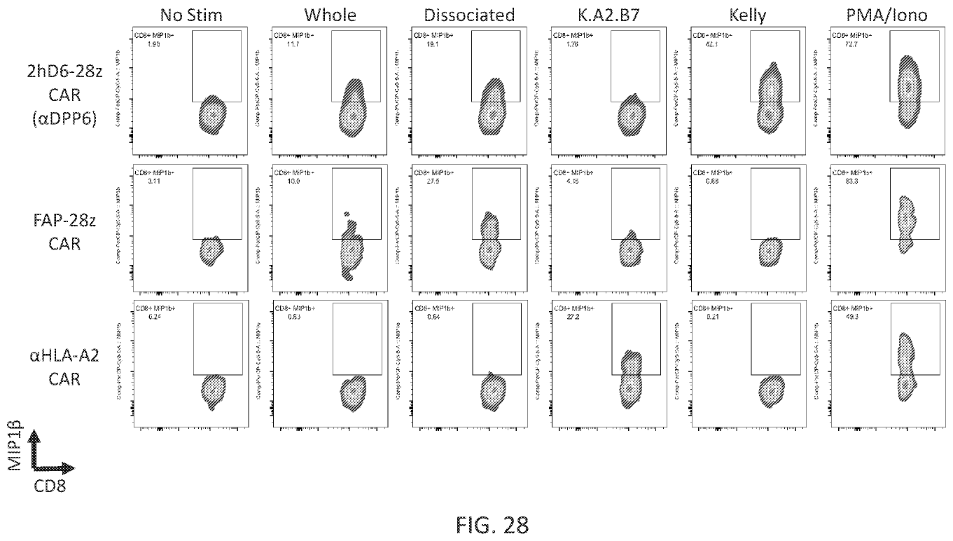

Two ways were developed which redirect T cells to the pancreatic islets in primate subjects: chimeric antigen receptors (CARs) specific for fibroblast activation protein (FAP) which is expressed on a cells and in areas of wound repair and dipeptidyl peptidase like 6 (DPP6), whose expression is limited to the brain and alpha and beta cells within the islet. Initial studies explored whether the human islet-specific CART cells described above would cross-react with non-human primate islet cells. Here, harvested islets from a non-human primate were mixed with control CAR (HLA-A2 specific), DPP6 and FAP-specific T cells. As shown in FIG. 28 both the DPP6 and FAP recognized the NHP islets whereas the control CAR T cells did not.

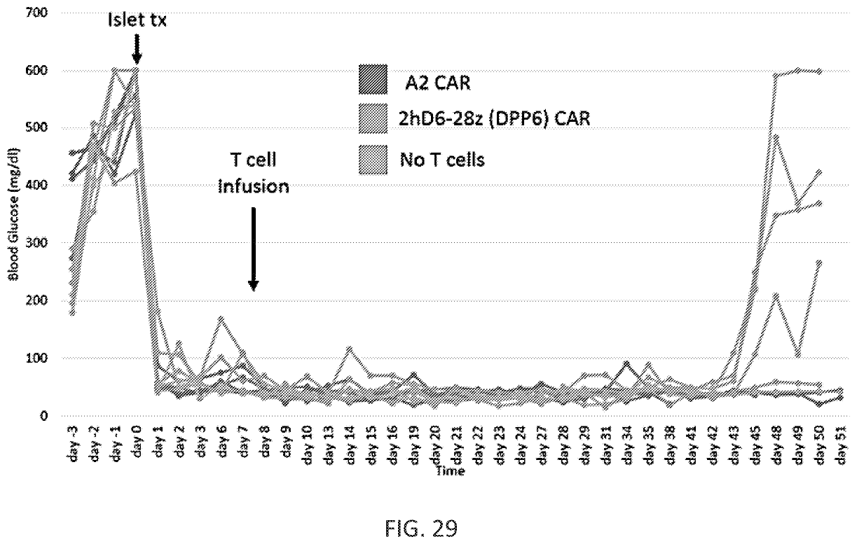

Next, islet cells isolated from non-human primate donors were transplanted into streptozotocin (STZ)-treated mice. After allowing engraftment and establishing that the transplanted cells had restored normal glycemic levels, animals were then infused with either control or DPP6-specific CAR T cells, as described previously in the present disclosure. After about 45 days, 4 out of 5 of the mice treated with DPP6-CAR T cells demonstrated hyperglycemia as measured by sampling peripheral blood, whereas glycemic levels remained unchanged in both non-transferred mice and those who received only control (HLA-A2) CAR T cells (FIG. 29). In total, and without wishing to be bound by theory, these data indicate that the islet-specific CAR T cells of the present invention are able to recognize and deplete non-human primate islet in vivo.

While preliminary studies were performed in mice with NHP islet cells, the CAR-based T1D NHP models of the present invention can also be induced in NHPs directly via the adoptive transfer of CART cells. Here, primate subjects are infused with a mixture of 1×10⁸ FAP and DPP6 CARs followed by observation of T1D induction using c-peptide levels and/or loss of insulin production after arginine infusion after one month. In certain instances, where T1D does not develop, a second infusion of CART cells is administered. Engraftment is assessed by assessing for CAR T cell infiltration into islets, brain and elsewhere in the body in order to determine the extent of CAR T cell localization to the islets or potential on target, off-tissue areas. FAP CAR T cells can be distinguished from the DPP6 cells using number of marker techniques so that the successful in infiltration of one cells over the other can be easily assessed. This model allows for the study of other factors/insults that are required to initiate and maintain islet cell destruction, especially given that recent studies have found that otherwise healthy people can possess populations of islet-specific T and B cells which are otherwise inhibited from autoreactivity. Infusion of islet specific CAR T cells can be accomplished by several routes, including intravenously and by infusion directly into the islets via the splenic artery. Other aspects of this NHP T1D model can be modified to assess T1D risk factors, including the disruption immune-regulating molecules such as PD-1 via CRISPR or antibody-blockade methods, or use suboptimal doses of STZ to induce islet beta cell injury sufficient promote CART cell mediated immune pathology. Overall, this model provides a robust and reproducible method to induce T1D in NHP that is engineered T cell dependent.

### Example 4: Use of Anti-FAP CAR Expressing Treg to Protect Islet Cells

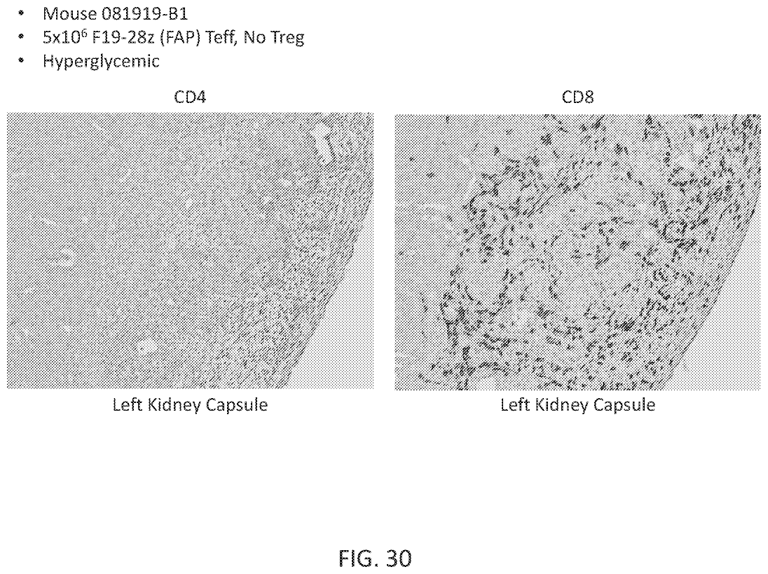

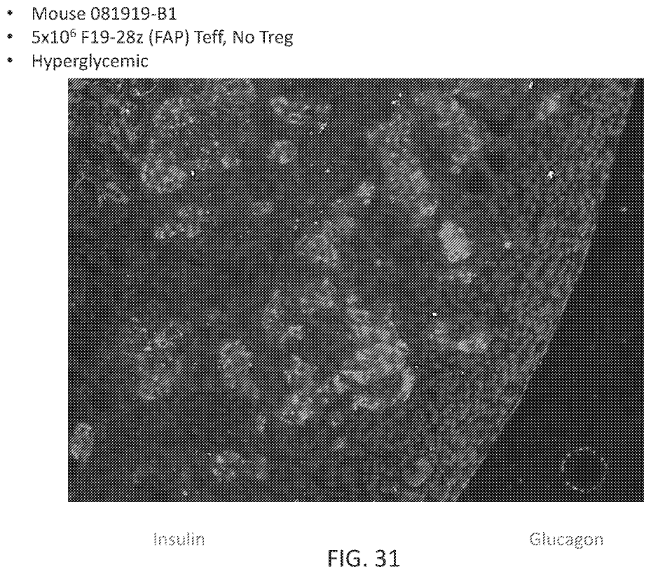

Recent studies have demonstrated that chimeric antigen receptor (CAR) constructs could be produced with antigen-binding domains specific for fibroblast activation protein (FAP), and that these FAP CAR constructs can be expressed in effector T cells and then directed against activated fibroblasts. See U.S. Pat. Nos. 9,365,641, 10,329,355, U.S. patent application Ser. No. 16/417,125, and U.S. patent application Ser. No. 16/651,144, the contents of which are incorporated herein. Still other studies have demonstrated that pancreatic alpha cells in adult humans express abundant amounts of FAP protein. As such, and without wishing to be bound by theory, it was hypothesized that CD4+ regulatory T cells expressing FAP CAR constructs could targeted to pancreatic islet tissue and thereby inhibit autoreactive T cell responses which cause T1D. In order to observe the ability of FAP CAR expressing T cells to traffic to islet cell tissue, mice were transplanted with human islet cells under the kidney capsule similar to studies previously described in the current disclosure which used DPP6 CAR T cells. After allowing for engraftment, mice were injected with FAP CAR expressing T cells followed my imaging. FIG. 30 demonstrates that FAP CAR T cells are able to infiltrate the kidney capsule into regions containing β-cells. For comparison, FIG. 31 is a micrograph from the same mouse showing that FAP CAR infiltration occurs in areas of islet cell engraftment.

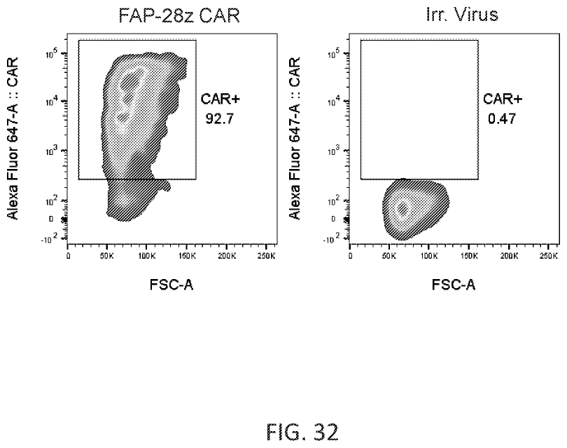

The ability of human CD4+ regulatory T cells to be transduced with FAP CAR constructs was then investigated. FIG. 32 illustrates human Tregs transduced with FAP-28z CAR or irrelevant CAR virus and stained for CAR construct expression via flow cytometry.

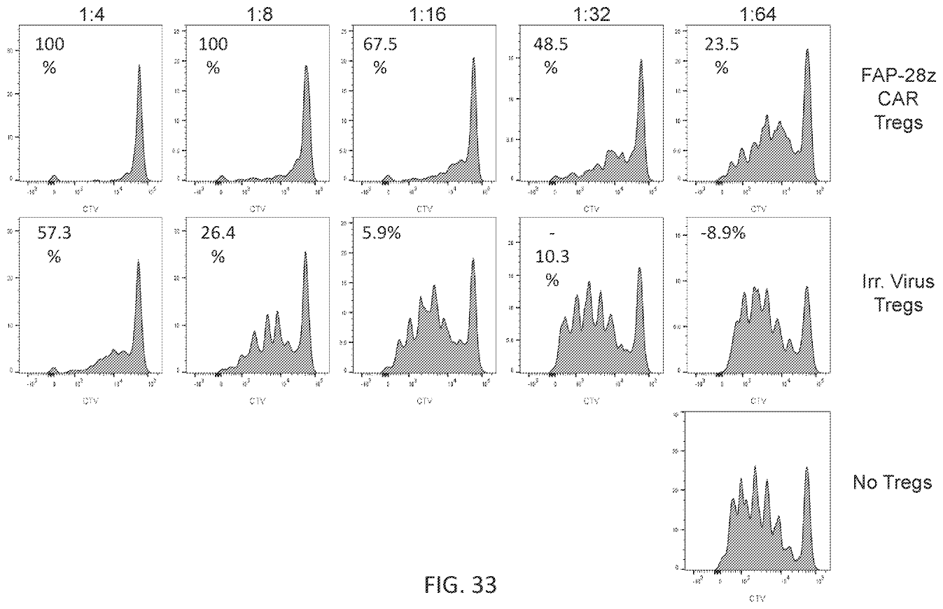

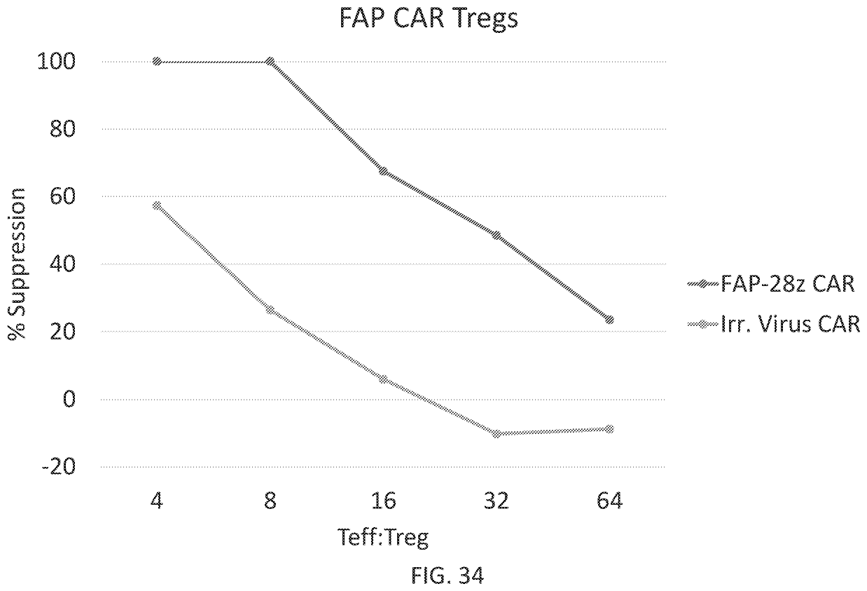

A series of in vitro suppression assays were then performed using FAP CAR expressing Tregs as suppressor cells (FIG. 33). Human Tregs transduced to express the FAP-28z CAR or and irrelevant CAR were co-cultured with CellTrace Violet responder cells and aAPCs expressing FAP. Results demonstrated that FAP CAR expressing T regs were able to suppress responder cells in an antigen-specific manner. FIG. 34 illustrates the calculated percent suppression for these assays, further demonstrating the suppressive ability of FAP CAR Treg cells. In total, and without wishing to be bound by theory, these results demonstrate that FAP CAR expressing Treg cells can traffic to islet cell tissues and exert suppressive function, suggesting their use to treat T1D.

### ENUMERATED EMBODIMENTS

The following enumerated embodiments are provided, the numbering of which is not to be construed as designating levels of importance.

Embodiment 1 provides a modified immune cell or precursor cell thereof, comprising a chimeric antigen receptor (CAR) having affinity for DPP6, wherein the CAR comprises a DPP6 binding domain, a transmembrane domain, and an intracellular domain.

Embodiment 2 provides the modified cell of embodiment 1, wherein the DPP6 binding domain is a nanobody.

Embodiment 3 provides the modified cell of embodiment 1, wherein the DPP6 binding domain comprises a heavy chain variable region comprising a first CDR region comprising an amino acid sequence selected from the group set forth in SEQ ID NOs: 3, 19, 28, and 35; a second CDR region comprising an amino acid sequence selected from the group set forth in SEQ ID NOs: 4, 20, 29, and 36; and a third CDR region comprising an amino acid sequence selected from the group consisting of AT and set forth in SEQ ID NOs: 21, 30, and 37.

Embodiment 4 provides the modified cell of embodiment 1, wherein the DPP6 binding domain comprises a heavy chain variable region comprising the amino acid sequence set forth in SEQ ID NOs: 1, 17, 26, and 33.

Embodiment 5 provides the modified cell of embodiment 1, wherein the CAR further comprises a hinge domain.

Embodiment 6 provides the modified cell of embodiment 5, wherein the hinge domain comprises a CD8 hinge.

Embodiment 7 provides the modified cell of embodiment 6, wherein the CD8 hinge comprises the amino acid sequence set forth in SEQ ID NO: 22.

Embodiment 8 provides the modified cell of embodiment 1, wherein the CAR further comprises a spacer domain.

Embodiment 9 provides the modified cell of embodiment 8, wherein the spacer domain is a human IgG4 spacer domain.

Embodiment 10 provides the modified cell of embodiment 9, wherein the human IgG4 spacer domain comprises the amino acid sequence set forth in SEQ ID NO: 7.

Embodiment 11 provides the modified cell of embodiment 1, wherein the transmembrane domain comprises a CD28 transmembrane domain.

Embodiment 12 provides the modified cell of embodiment 11, wherein the transmembrane domain comprises the amino acid sequence set forth in SEQ ID NO: 10.

Embodiment 13 provides the modified cell of embodiment 1, wherein the intracellular domain comprises a CD28 costimulatory domain.

Embodiment 14 provides the modified cell of embodiment 13, wherein the CD28 costimulatory domain comprises the amino acid sequence set forth in SEQ ID NO: 11.

Embodiment 15 provides the modified cell of embodiment 1, wherein the intracellular domain comprises a CD3 domain.

Embodiment 16 provides the modified cell of embodiment 15, wherein the CD3 □ domain comprises the amino acid sequence set forth in SEQ ID NO: 13.

Embodiment 17 provides the modified cell of embodiment 1, wherein the intracellular domain comprises a CD28 costimulatory domain and a CD3 domain.

Embodiment 18 provides the modified cell of embodiment 1, wherein the CAR further comprises a CD8 signal peptide.

Embodiment 19 provides the modified cell of embodiment 18, wherein the signal peptide comprises the amino acid sequence set forth in SEQ ID NO: 5.

Embodiment 20 provides a modified immune cell or precursor cell thereof, comprising a chimeric antigen receptor (CAR) having affinity for DPP6, wherein the CAR comprises an DPP6 binding domain, a CD8 hinge domain, a CD28 transmembrane domain, a CD28 costimulatory domain, and a CD3 intracellular domain.

Embodiment 21 provides a modified immune cell or precursor cell thereof, comprising a chimeric antigen receptor (CAR) having affinity for DPP6, wherein the CAR comprises an DPP6 binding domain, a human IgG4 hinge domain, a CD28 transmembrane domain, a CD28 costimulatory domain, and a CD3 intracellular domain.

Embodiment 22 provides the modified cell of any preceding embodiment, wherein the CAR comprises the amino acid sequence set forth in SEQ ID NOs: 15, 24, 31, and 38.

Embodiment 23 provides the modified cell of any preceding embodiment, wherein the modified cell is a regulatory T cell.

Embodiment 24 provides the modified cell of any preceding embodiment, wherein the modified cell is an autologous cell.

Embodiment 25. Provides the modified cell of any preceding embodiment, wherein the modified cell is derived from a human.

Embodiment 26 provides an isolated nucleic acid, comprising a nucleic acid sequence encoding a chimeric antigen receptor (CAR) having affinity for DPP6, wherein the CAR comprises a DPP6 binding domain, a transmembrane domain, and an intracellular domain.

Embodiment 27 provides the isolated nucleic acid of embodiment 26, wherein the DPP6 binding domain comprises a nanobody.

Embodiment 28 provides the isolated nucleic acid of embodiment 26, wherein the DPP6 binding domain comprises a heavy chain variable region comprising a first CDR region comprising an amino acid sequence selected from the group set forth in SEQ ID NOs: 3, 19, 28, and 35; a second CDR region comprising an amino acid sequence selected from the group set forth in SEQ ID NOs: 4, 20, 29, and 36; and a third CDR region comprising an amino acid sequence selected from the group consisting of AT and set forth in SEQ ID NOs: 21, 30, and 37.

Embodiment 29 provides the isolated nucleic acid of embodiment 26, wherein the DPP6 binding domain comprises a heavy chain variable region comprising a nucleic acid sequence set forth in SEQ ID NOs: 2, 18, 27, and 34.

Embodiment 30 provides the isolated nucleic acid of embodiment 26, wherein the CAR comprises a CD28 transmembrane domain.

Embodiment 31 provides the isolated nucleic acid of embodiment 30, wherein the CD28 transmembrane domain comprises a nucleic acid sequence set forth in SEQ ID NO: 9.

Embodiment 32 provides the isolated nucleic acid of embodiment 26, wherein the intracellular domain comprises a CD28 costimulatory domain.

Embodiment 33 provides the isolated nucleic acid of embodiment 32, wherein the CD28 costimulatory domain comprises a nucleic acid sequence set forth in SEQ ID NO: 12.

Embodiment 34 provides the isolated nucleic acid of embodiment 26, wherein the intracellular domain comprises a CD3 domain.

Embodiment 35 provides the isolated nucleic acid of embodiment 34, wherein the CD3 domain comprises a nucleic acid sequence set forth in SEQ ID NO: 14.

Embodiment 36 provides the isolated nucleic acid of embodiment 26, comprising a nucleic acid sequence selected from the group set forth in SEQ ID NOs: 16, 25, 32, and 39.

Embodiment 37 provides an expression construct comprising the isolated nucleic acid of embodiment 26.

Embodiment 38 provides a method for generating the modified immune cell or precursor cell thereof of embodiment 1, comprising introducing into the immune cell the nucleic acid of any one of embodiments 26-36, or the expression construct of embodiment 37.

Embodiment 39 provides a method of treating an autoimmune disease in a subject in need thereof, comprising administering to the subject an effective amount of the modified immune cell or precursor cell thereof of any one of embodiments 1-25.

Embodiment 40 provides the method of embodiment 39, wherein the autoimmune disease is type 1 diabetes.

Embodiment 41 provides a method of treating type 1 diabetes in a subject in need thereof, comprising administering to the subject a modified regulatory T cell comprising a chimeric antigen receptor (CAR) having affinity for DPP6, wherein the CAR comprises an DPP6 binding domain, a CD8 hinge domain, a CD28 transmembrane domain, a CD28 costimulatory domain, and a CD3 intracellular domain.

Embodiment 42 provides a method of treating type 1 diabetes in a subject in need thereof, comprising administering to the subject a modified regulatory T cell comprising a chimeric antigen receptor (CAR) having affinity for DPP6, wherein the CAR comprises an DPP6 binding domain, a human IgG4 hinge domain, a CD28 transmembrane domain, a CD28 costimulatory domain, and a CD3 intracellular domain.

Embodiment 43 provides the method of any one of embodiments 41 and 42, wherein the modified cell is a modified regulatory T cell.

Embodiment 44 provides the method of any one of embodiments 41 and 42, wherein the modified cell is an autologous cell.

Embodiment 45 provides the method of any one of embodiments 41 and 42, wherein the modified cell is derived from a human.

Embodiment 46 provides a non-human primate model of type 1 diabetes, comprising administering to a non-human primate subject an effective amount of a modified T cell comprising a chimeric antigen receptor (CAR) having an affinity for a islet cell antigen.

Embodiment 47 provides the non-human primate model of embodiment 46, wherein the CAR has an affinity for DPP6.

Embodiment 48 provides the non-human primate model of embodiment 46, wherein the CAR has an affinity for fibroblast activation protein (FAP).

Embodiment 49 provides a method of generating a non-human primate model of type 1 diabetes, the method comprising administering to a non-human primate subject an effective amount of a first modified T cell comprising a CAR having an affinity for an islet cell antigen and an effective amount of a second modified T cell comprising a CAR having an affinity for a different islet cell antigen.

Embodiment 50 provides the non-human primate model of embodiment 49, wherein the first islet cell antigen is DPP6 and the second islet cell antigen is FAP.

Embodiment 51 provides the non-human primate model of any one of embodiments 46-50, wherein the modified T cells are administered intravenously.

Embodiment 52 provides the non-human primate model of any one of embodiments 46-50, wherein the modified T cells are administered via the splenic artery.

Embodiment 53 provides the non-human primate model of any one of embodiments 46-52, further comprising the administration of streptozotocin, wherein the amount of streptozotocin is sufficient to induce islet cell injury but not depletion.

Embodiment 54 provides the non-human primate model of any one of embodiments 46-53, further comprising the administration of an effective amount of an immune-modulating agent.

Embodiment 55 provides the non-human primate model of embodiment 54, wherein the immune-modulating agent is a CRISPR-based system.

Embodiment 56 provides the non-human primate model of embodiment 55, wherein the CRISPR-based system disrupts the expression of an immune checkpoint protein.

Embodiment 57 provides the non-human primate model of embodiment 56, wherein the immune checkpoint protein is selected from the group consisting of PD-1, CTLA-4, TIM3, GITR, BTLA, LAG3, and any combination thereof.

Embodiment 58 provides the non-human primate model of embodiment 56, wherein the subject is selected from the group consisting of a rhesus macaque, a cynomolgus macaque, a chimpanzee, and a baboon.

Embodiment 59 provides a non-human primate animal model of diabetes made by the method of any of embodiments 46-58.

Embodiment 60 provides a method of treating type 1 diabetes in a subject in need thereof, comprising administering to the subject a modified regulatory T cell comprising a chimeric antigen receptor (CAR) having affinity for FAP, wherein the CAR comprises an FAP binding domain, a CD28 transmembrane domain, a CD28 costimulatory domain, and a CD3ζ intracellular domain.

Embodiment 61 provides the method of embodiment 60, wherein the modified cell is an autologous cell.

Embodiment 62 provides the method of embodiment 60, wherein the modified cell is derived from a human.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A modified immune cell or precursor cell thereof, comprising a chimeric antigen receptor (CAR) having affinity for DPP6, wherein the CAR comprises a DPP6 binding domain, a transmembrane domain, and an intracellular domains
wherein the DPP6 binding domain comprises a variable region comprising a first CDR region comprising an amino acid sequence set forth in SEQ ID NO: 35; a second CDR region comprising an amino acid sequence set forth in SEQ ID NO: 36; and a third CDR region comprising an amino acid sequence set forth in SEQ ID NO: 37.

2. The modified cell of claim 1, wherein the DPP6 binding domain is a nanobody.

3. (canceled)

4. The modified cell of claim 1, wherein the DPP6 binding domain comprises a variable region comprising the amino acid sequence set forth in SEQ ID NO: 33.

5. The modified cell of claim 1, wherein the CAR further comprises a hinge domain.

6. The modified cell of claim 5, wherein the hinge domain comprises a CD8 hinge.

7. The modified cell of claim 6, wherein the CD8 hinge comprises the amino acid sequence set forth in SEQ ID NO: 22.

8. The modified cell of claim 1, wherein the CAR further comprises a spacer domain.

9. The modified cell of claim 8, wherein the spacer domain is a human IgG4 spacer domain.

10. The modified cell of claim 9, wherein the human IgG4 spacer domain comprises the amino acid sequence set forth in SEQ ID NO: 7.

11. The modified cell of claim 1, wherein the transmembrane domain comprises a CD28 transmembrane domain.

12. The modified cell of claim 11, wherein the transmembrane domain comprises the amino acid sequence set forth in SEQ ID NO: 10.

13. The modified cell of claim 1, wherein the intracellular domain comprises a CD28 costimulatory domain.

14. The modified cell of claim 13, wherein the CD28 costimulatory domain comprises the amino acid sequence set forth in SEQ ID NO: 11.

15. The modified cell of claim 1, wherein the intracellular domain comprises a CD3ζ domain.

16. The modified cell of claim 15, wherein the CD3ζ domain comprises the amino acid sequence set forth in SEQ ID NO: 13.

17. The modified cell of claim 1, wherein the intracellular domain comprises a CD28 costimulatory domain and a CD3ζ domain.

18. The modified cell of claim 1, wherein the CAR further comprises a CD8 signal peptide.

19. The modified cell of claim 18, wherein the signal peptide comprises the amino acid sequence set forth in SEQ ID NO: 5.

20. A modified immune cell or precursor cell thereof, comprising a chimeric antigen receptor (CAR) having affinity for DPP6, wherein the CAR comprises an DPP6 binding domain, a hinge domain, a CD28 transmembrane domain, a CD28 costimulatory domain, and a CD3ζ intracellular domain;
wherein in the hinge domain is selected from the group consisting of a CD8 hinge domain and an IgG4 hinge domain; and
wherein the DPPP6 binding domain comprises a variable region comprising a first CDR region comprising an amino acid sequence set forth in SEQ ID NO: 35; a second CDR region comprising an amino acid sequence set forth in SEQ ID NO: 36; and a third CDR region comprising an amino acid sequence set forth in SEQ ID NO: 37.

21. (canceled)

22. The modified cell of claim 20, wherein the CAR comprises the amino acid sequence set forth in SEQ ID NOs: 38.

23. The modified cell of claim 20, wherein the modified cell is a regulatory T cell.

24. The modified cell of claim 20, wherein the modified cell is an autologous cell.

25. The modified cell of claim 20, wherein the modified cell is derived from a human.

26. An isolated nucleic acid, comprising a nucleic acid sequence encoding a chimeric antigen receptor (CAR) having affinity for DPP6, wherein the CAR comprises a DPP6 binding domain, a transmembrane domain, and an intracellular domain; and
wherein the DPP6 binding domain comprises a variable region comprising a first CDR region comprising an amino acid sequence set forth in SEQ ID NO: 35; a second CDR region comprising an amino acid sequence set forth in SEQ ID NO: 36; and a third CDR region comprising an amino acid sequence set forth in SEQ ID NO: 37.

27. The isolated nucleic acid of claim 26, wherein the DPP6 binding domain comprises a nanobody.

28. (canceled)

29. The isolated nucleic acid of claim 26, wherein the DPP6 binding domain comprises a variable region comprising a nucleic acid sequence set forth in SEQ ID NO: 34.

30. The isolated nucleic acid of claim 26, wherein the CAR comprises a CD28 transmembrane domain.

31. The isolated nucleic acid of claim 30, wherein the CD28 transmembrane domain comprises a nucleic acid sequence set forth in SEQ ID NO: 9.

32. The isolated nucleic acid of claim 26, wherein the intracellular domain comprises a CD28 costimulatory domain.

33. The isolated nucleic acid of claim 32, wherein the CD28 costimulatory domain comprises a nucleic acid sequence set forth in SEQ ID NO: 12.

34. The isolated nucleic acid of claim 26, wherein the intracellular domain comprises a CD3ζ domain.

35. The isolated nucleic acid of claim 34, wherein the CD3ζ domain comprises a nucleic acid sequence set forth in SEQ ID NO: 14.

36. The isolated nucleic acid of claim 26, comprising a nucleic acid sequence selected from the group set forth in SEQ ID NO: 39.

37. An expression construct comprising the isolated nucleic acid of claim 26.

38. A method for generating a modified immune cell or precursor cell thereof, comprising introducing into the immune cell the nucleic acid of claim 26.

39. A method of treating an autoimmune disease in a subject in need thereof, comprising administering to the subject an effective amount of the modified immune cell or precursor cell thereof of claim 1.

40. The method of claim 39, wherein the autoimmune disease is type 1 diabetes.

41. A method of treating type 1 diabetes in a subject in need thereof, comprising administering to the subject a modified T cell comprising a chimeric antigen receptor (CAR) having affinity for DPP6, wherein the CAR comprises an DPP6 binding domain, a hinge domain, a CD28 transmembrane domain, a CD28 costimulatory domain, and a CD3ζ intracellular domain;
wherein the hinge domain is selected from the group consisting of a CD8 hinge domain and an IgG4 hinge domain; and
wherein the DPP6 binding domain comprises a variable region comprising a first CDR region comprising an amino acid sequence set forth in SEQ ID NO: 35; a second CDR region comprising an amino acid sequence set forth in SEQ ID NO: 36; and a third CDR region comprising an amino acid sequence set forth in SEQ ID NO: 37.

42. (canceled)

43. The method of claim 41, wherein the modified T cell is a modified regulatory T cell.

44. The method of claim 41, wherein the modified T cell is an autologous cell.

45. The method of claim 41, wherein the modified T cell is derived from a human.

46. A method of generating a non-human primate model of type 1 diabetes, the method comprising administering to a non-human primate subject an effective amount of a modified T cell comprising a chimeric antigen receptor (CAR) having an affinity for a islet cell antigen.

47. The method of claim 46, wherein the CAR has an affinity for DPP6.

48. The method of claim 46, wherein the CAR has an affinity for fibroblast activation protein (FAP).

49. The method of claim 46, further comprising administering to the non-human primate subject an effective amount of a second modified T cell comprising a CAR having an affinity for a different islet cell antigen.

50. The method of claim 49, wherein the islet cell antigens are DPP6 and FAP.

51. The method of claim 46, wherein the modified T cells are administered intravenously.

52. The method of claim 46, wherein the modified T cells are administered via the splenic artery.

53. The method of claim 46, further comprising administering an effective amount of streptozotocin to the non-human primate subject, wherein the amount of streptozotocin is sufficient to induce islet cell injury but not depletion.

54. The method of claim 46, further comprising administering an effective amount of an immune-modulating agent to the non-human primate subject.

55. The method of claim 54, wherein the immune-modulating agent is a CRISPR-based system.

56. The method of claim 55, wherein the CRISPR-based system disrupts the expression of an immune checkpoint protein.

57. The method of claim 56, wherein the immune checkpoint protein is selected from the group consisting of PD-1, CTLA-4, TIM3, GITR, BTLA, LAG3, and any combination thereof.

58. The method of claim 46, wherein the subject is selected from the group consisting of a rhesus macaque, a cynomolgus macaque, a chimpanzee, and a baboon.

59. A non-human primate animal model of diabetes made by the method of claim 46.

60. A method of treating type 1 diabetes in a subject in need thereof, comprising administering to the subject a modified regulatory T cell comprising a chimeric antigen receptor (CAR) having affinity for FAP, wherein the CAR comprises an FAP binding domain, a CD28 transmembrane domain, a CD28 costimulatory domain, and a CD3ζ intracellular domain.

61. The method of claim 60, wherein the modified cell is an autologous cell.

62. The method of claim 60, wherein the modified cell is derived from a human.


SEQUENCE LISTING (64 sequences)

SEQ ID NO: 1 - protein, 119 aa, Artificial Sequence
  4hD29 nanobody aa
QVQLQESGGGLVQPGGSLRLSCAASGFTFSSNYMTWVRQAPGKGPEWVSGINPDGSSTYYADSVKGRFTISRDNAKNTLYLQMNSLKSEDTALYKCATGAAPRIPTTLRGQGTQVTVSS

SEQ ID NO: 2 - DNA, 357 bp, Artificial Sequence
  4hD29 nanobody
caagtgcaacttcaagagtccggcggtggacttgtccagccaggagggagcctgcgactttcctgcgccgccagtgggttcacattttccagtaattatatgacttgggtacgacaggcacctggcaaaggccctgagtgggtctcaggaattaacccggatggttccagcacatactacgcggattctgttaagggccgctttaccatctcaagggataacgctaagaataccctttatctccagatgaacagtctcaaaagcgaggacaccgcattgtataaatgtgcaacaggggctgctcctaggattccaaccacattgagaggtcaaggcactcaggtgactgtctcctca

SEQ ID NO: 3 - protein, 8 aa, Artificial Sequence
  4hD29 CDR1
GFTFSSNY

SEQ ID NO: 4 - protein, 8 aa, Artificial Sequence
  4hD29 CDR2
INPDGSST

SEQ ID NO: 5 - protein, 21 aa, Artificial Sequence
  CD8 signal peptide aa
MALPVTALLLPLALLLHAARP

SEQ ID NO: 6 - DNA, 63 bp, Artificial Sequence
  CD8 signal peptide
atggccttaccagtgaccgccttgctcctgccgctggccttgctgctccacgccgccaggccg

SEQ ID NO: 7 - protein, 229 aa, Artificial Sequence
  IgG4 hinge aa
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - DNA, 687 bp, Artificial Sequence
  IgG4 hinge
gagagcaagtacggccccccttgccccagctgtccggcaccagagttcgaagggggaccctccgtctttctcttcccccctaagcccaaggataccctgatgatttcacgcactcctgaggtaacttgcgtggtcgtagatgttagccaggaagatccagaagttcagtttaattggtacgttgatggggtggaggtgcacaatgccaaaactaaacccagagaggaacagttccagtctacttatcgagtggttagcgttctgaccgtcctgcaccaggactggctgaacggaaaggaatataaatgcaaggtcagtaacaaggggctccctagctccatcgagaaaacgatatcaaaagcaaagggacagccaagagagcctcaggtctacactcttcctccaagccaggaagaaatgaccaaaaaccaggtttcccttacctgtctggttaagggcttttacccatccgatattgcggtggaatgggaatccaacggtcaaccggaaaacaattacaagaccacccctccggtcctggacagcgatggaagtttttttttgtattccaggctcaccgtcgataaaagtaggtggcaggaaggaaacgtcttttcctgcagtgtgatgcatgaagcgctgcataatcattatacacagaagtccttgagtcttagcctcgggaaa

SEQ ID NO: 9 - DNA, 81 bp, Artificial Sequence
  CD28 transmembrane domain
ttttgggtgctggtggtggttggtggagtcctggcttgctatagcttgctagtaacagtggcctttattattttctgggtg

SEQ ID NO: 10 - protein, 27 aa, Artificial Sequence
  CD28 transmembrane domain aa
FWVLVVVGGVLACYSLLVTVAFIIFWV

SEQ ID NO: 11 - protein, 41 aa, Artificial Sequence
  CD28 intracellular domain aa
RSKRSRLLHSDYMNMTPRRPGPTRKHYQPYAPPRDFAAYRS

SEQ ID NO: 12 - DNA, 123 bp, Artificial Sequence
  CD28 intracellular domain
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 13 - protein, 112 aa, Artificial Sequence
  CD3 zeta domain aa
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - DNA, 336 bp, Artificial Sequence
  CD3 zeta domain
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 15 - protein, 553 aa, Artificial Sequence
  4hD29-28z CAR aa
MALPVTALLLPLALLLHAARPGSQVQLQESGGGLVQPGGSLRLSCAASGFTFSSNYMTWVRQAPGKGPEWVSGINPDGSSTYYADSVKGRFTISRDNAKNTLYLQMNSLKSEDTALYKCATGAAPRIPTTLRGQGTQVTVSSESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVRSKRSRLLHSDYMNMTPRRPGPTRKHYQPYAPPRDFAAYRSIDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - DNA, 1662 bp, Artificial Sequence
  4hD29-28z CAR
atggccttaccagtgaccgccttgctcctgccgctggccttgctgctccacgccgccaggccgggatcccaagtgcaacttcaagagtccggcggtggacttgtccagccaggagggagcctgcgactttcctgcgccgccagtgggttcacattttccagtaattatatgacttgggtacgacaggcacctggcaaaggccctgagtgggtctcaggaattaacccggatggttccagcacatactacgcggattctgttaagggccgctttaccatctcaagggataacgctaagaataccctttatctccagatgaacagtctcaaaagcgaggacaccgcattgtataaatgtgcaacaggggctgctcctaggattccaaccacattgagaggtcaaggcactcaggtgactgtctcctcagagagcaagtacggccccccttgccccagctgtccggcaccagagttcgaagggggaccctccgtctttctcttcccccctaagcccaaggataccctgatgatttcacgcactcctgaggtaacttgcgtggtcgtagatgttagccaggaagatccagaagttcagtttaattggtacgttgatggggtggaggtgcacaatgccaaaactaaacccagagaggaacagttccagtctacttatcgagtggttagcgttctgaccgtcctgcaccaggactggctgaacggaaaggaatataaatgcaaggtcagtaacaaggggctccctagctccatcgagaaaacgatatcaaaagcaaagggacagccaagagagcctcaggtctacactcttcctccaagccaggaagaaatgaccaaaaaccaggtttcccttacctgtctggttaagggcttttacccatccgatattgcggtggaatgggaatccaacggtcaaccggaaaacaattacaagaccacccctccggtcctggacagcgatggaagtttttttttgtattccaggctcaccgtcgataaaagtaggtggcaggaaggaaacgtcttttcctgcagtgtgatgcatgaagcgctgcataatcattatacacagaagtccttgagtcttagcctcgggaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 17 - protein, 126 aa, Artificial Sequence
  2hD1 nanobody aa
QVQLQESGGGLVQPGGSLRLSCAASGYPYGYTFSSYCMRWFRQAPGKDREGVARFERNGLTTYYDDSVKGRFTISQDNVKNTVYLQMNSLKPEDTATYYCAAAPKQLRTCGDYNYWGQGTQVTVSS

SEQ ID NO: 18 - DNA, 378 bp, Artificial Sequence
  2hD1 nanobody
caggttcaattgcaagagtccggcggcgggcttgtgcaaccaggcggcagcttgcgcctctcatgtgctgcatccggttacccatatggctacacgtttagttcttactgtatgcgatggttccggcaggcgcctgggaaggatcgggaaggggttgctaggtttgaaagaaacggacttaccacttactatgacgactccgttaaaggtagatttactatatcccaggacaatgtgaaaaataccgtgtacttgcaaatgaatagcttgaaaccagaggacactgcaacatactattgcgctgctgccccaaaacaactgaggacgtgtggggattacaactattgggggcaaggcactcaagtcactgtcagttct

SEQ ID NO: 19 - protein, 8 aa, Artificial Sequence
  2hD1 CDR1
GYTFSSYC

SEQ ID NO: 20 - protein, 8 aa, Artificial Sequence
  2hD1 CDR2
FERNGLTT

SEQ ID NO: 21 - protein, 15 aa, Artificial Sequence
  2hD1 CDR3
AAAPKQLRTCGDYNY

SEQ ID NO: 22 - protein, 45 aa, Artificial Sequence
  CD8 hinge aa
TTTPAPRPPTPAPTIASQPLSLRPEACRPAAGGAVHTRGLDFACD

SEQ ID NO: 23 - DNA, 135 bp, Artificial Sequence
  CD8 hinge
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 24 - protein, 378 aa, Artificial Sequence
  2hD1-28z CAR aa
MALPVTALLLPLALLLHAARPGSQVQLQESGGGLVQPGGSLRLSCAASGYPYGYTFSSYCMRWFRQAPGKDREGVARFERNGLTTYYDDSVKGRFTISQDNVKNTVYLQMNSLKPEDTATYYCAAAPKQLRTCGDYNYWGQGTQVTVSSSGTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSIDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - DNA, 1137 bp, Artificial Sequence
  2hD1-28z CAR
atggccttaccagtgaccgccttgctcctgccgctggccttgctgctccacgccgccaggccgggatcccaggttcaattgcaagagtccggcggcgggcttgtgcaaccaggcggcagcttgcgcctctcatgtgctgcatccggttacccatatggctacacgtttagttcttactgtatgcgatggttccggcaggcgcctgggaaggatcgggaaggggttgctaggtttgaaagaaacggacttaccacttactatgacgactccgttaaaggtagatttactatatcccaggacaatgtgaaaaataccgtgtacttgcaaatgaatagcttgaaaccagaggacactgcaacatactattgcgctgctgccccaaaacaactgaggacgtgtggggattacaactattgggggcaaggcactcaagtcactgtcagttcttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 26 - protein, 130 aa, Artificial Sequence
  2hD6 nanobody aa
QVQLQESGGGSVQAGGSLRLSCAASSYTYSYSCMAWFRQAPGKERERVASIHTGTGTANYADSAKGRFTISQDIAANTVYLQMNSLKLEDTAMYYCAARPGSAALRCTTDYSKPHDFTYWGQGTQVTVSS

SEQ ID NO: 27 - DNA, 390 bp, Artificial Sequence
  2hD6 nanobody
caggtccaactccaggaatccggtggtgggtctgtgcaggcagggggcagtctgaggttgtcatgtgccgctagttcatacacatactcttactcttgtatggcctggtttcgacaggcacccggcaaagagcgggagagagttgccagcattcacacaggaactggaaccgccaattatgcagattctgctaagggtcggtttacaataagccaggatattgcagcgaataccgtatatttgcaaatgaacagccttaagctcgaagatactgcaatgtattactgcgctgcacgaccagggtctgctgcccttaggtgtacgactgattatagcaagccgcatgattttacttactgggggcaggggacacaggttactgtatcctct

SEQ ID NO: 28 - protein, 8 aa, Artificial Sequence
  2hD6 CDR1
SYTYSYSC

SEQ ID NO: 29 - protein, 8 aa, Artificial Sequence
  2hD6 CDR2
IHTGTGTA

SEQ ID NO: 30 - protein, 23 aa, Artificial Sequence
  2hD6 CDR3
AARPGSAALRCTTDYSKPHDFTY

SEQ ID NO: 31 - protein, 382 aa, Artificial Sequence
  2hD6-28z CAR aa
MALPVTALLLPLALLLHAARPGSQVQLQESGGGSVQAGGSLRLSCAASSYTYSYSCMAWFRQAPGKERERVASIHTGTGTANYADSAKGRFTISQDIAANTVYLQMNSLKLEDTAMYYCAARPGSAALRCTTDYSKPHDFTYWGQGTQVTVSSSGTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSIDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - DNA, 1149 bp, Artificial Sequence
  2hD6-28z CAR
atggccttaccagtgaccgccttgctcctgccgctggccttgctgctccacgccgccaggccgggatcccaggtccaactccaggaatccggtggtgggtctgtgcaggcagggggcagtctgaggttgtcatgtgccgctagttcatacacatactcttactcttgtatggcctggtttcgacaggcacccggcaaagagcgggagagagttgccagcattcacacaggaactggaaccgccaattatgcagattctgctaagggtcggtttacaataagccaggatattgcagcgaataccgtatatttgcaaatgaacagccttaagctcgaagatactgcaatgtattactgcgctgcacgaccagggtctgctgcccttaggtgtacgactgattatagcaagccgcatgattttacttactgggggcaggggacacaggttactgtatcctcttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 33 - protein, 128 aa, Artificial Sequence
  2hD38 nanobody aa
QVQLQESGGGSVQAGGSLRLSCAASENTQGNYCLAWFRQAPGKEREGVASISSGGIKTYYADTVKGRFTISRDNAENTVYLQMNSLKPEDTAIYYCAARTSVTCFASSWARLNAYAYWGQGTQVTVSS

SEQ ID NO: 34 - DNA, 384 bp, Artificial Sequence
  2hD38 nanobody
caggtgcaacttcaagaatcaggagggggttccgttcaagccggtgggtcactgaggctttcttgcgctgcttcagaaaacactcagggcaactactgcctcgcgtggtttcgacaagcaccagggaaggaacgggaaggagtggcttccataagcagcggaggaatcaaaacatactacgctgacaccgttaagggccggttcaccatcagccgcgataatgccgaaaatactgtgtacttgcagatgaattccctcaaaccagaggatacggctatttactactgtgcagcacggaccagcgtgacctgcttcgcctcctcatgggctaggctcaacgcttacgcctattggggacagggaactcaggtcacagtaagcagc

SEQ ID NO: 35 - protein, 8 aa, Artificial Sequence
  2hD38 CDR1
ENTQGNYC

SEQ ID NO: 36 - protein, 8 aa, Artificial Sequence
  2hD38 CDR2
ISSGGIKT

SEQ ID NO: 37 - protein, 21 aa, Artificial Sequence
  2hD38 CDR3
AARTSVTCFASSWARLNAYAY

SEQ ID NO: 38 - protein, 380 aa, Artificial Sequence
  2hD38-28z CAR aa
MALPVTALLLPLALLLHAARPGSQVQLQESGGGSVQAGGSLRLSCAASENTQGNYCLAWFRQAPGKEREGVASISSGGIKTYYADTVKGRFTISRDNAENTVYLQMNSLKPEDTAIYYCAARTSVTCFASSWARLNAYAYWGQGTQVTVSSSGTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSIDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - DNA, 1143 bp, Artificial Sequence
  2hD38-28z CAR
atggccttaccagtgaccgccttgctcctgccgctggccttgctgctccacgccgccaggccgggatcccaggtgcaacttcaagaatcaggagggggttccgttcaagccggtgggtcactgaggctttcttgcgctgcttcagaaaacactcagggcaactactgcctcgcgtggtttcgacaagcaccagggaaggaacgggaaggagtggcttccataagcagcggaggaatcaaaacatactacgctgacaccgttaagggccggttcaccatcagccgcgataatgccgaaaatactgtgtacttgcagatgaattccctcaaaccagaggatacggctatttactactgtgcagcacggaccagcgtgacctgcttcgcctcctcatgggctaggctcaacgcttacgcctattggggacagggaactcaggtcacagtaagcagctccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 40 - protein, 5 aa, Artificial Sequence
  Linker
GSGGS

SEQ ID NO: 41 - protein, 4 aa, Artificial Sequence
  Linker
GGGS

SEQ ID NO: 42 - protein, 5 aa, Artificial Sequence
  Linker
GGGGS

SEQ ID NO: 43 - protein, 4 aa, Artificial Sequence
  Linker
GGSG

SEQ ID NO: 44 - protein, 5 aa, Artificial Sequence
  Linker
GGSGG

SEQ ID NO: 45 - protein, 5 aa, Artificial Sequence
  Linker
GSGSG

SEQ ID NO: 46 - protein, 5 aa, Artificial Sequence
  Linker
GSGGG

SEQ ID NO: 47 - protein, 5 aa, Artificial Sequence
  Linker
GGGSG

SEQ ID NO: 48 - protein, 5 aa, Artificial Sequence
  Linker
GSSSG

SEQ ID NO: 49 - protein, 5 aa, Artificial Sequence
  Linker
GGGGS

SEQ ID NO: 50 - protein, 15 aa, Artificial Sequence
  Linker
GGGGSGGGGSGGGGS

SEQ ID NO: 51 - protein, 45 aa, Artificial Sequence
  Linker
GGTGGCGGTGGCTCGGGCGGTGGTGGGTCGGGTGGCGGCGGATCT

SEQ ID NO: 52 - protein, 5 aa, Artificial Sequence
  Hinge
DKTHT

SEQ ID NO: 53 - protein, 4 aa, Artificial Sequence
  Hinge
CPPC

SEQ ID NO: 54 - protein, 15 aa, Artificial Sequence
  Hinge
CPEPKSCDTPPPCPR

SEQ ID NO: 55 - protein, 12 aa, Artificial Sequence
  Hinge
ELKTPLGDTTHT

SEQ ID NO: 56 - protein, 10 aa, Artificial Sequence
  Hinge
KSCDKTHTCP

SEQ ID NO: 57 - protein, 7 aa, Artificial Sequence
  Hinge
KCCVDCP

SEQ ID NO: 58 - protein, 7 aa, Artificial Sequence
  Hinge
KYGPPCP

SEQ ID NO: 59 - protein, 15 aa, Artificial Sequence
  Hinge
EPKSCDKTHTCPPCP

SEQ ID NO: 60 - protein, 12 aa, Artificial Sequence
  Hinge
ERKCCVECPPCP

SEQ ID NO: 61 - protein, 17 aa, Artificial Sequence
  Hinge
ELKTPLGDTTHTCPRCP

SEQ ID NO: 62 - protein, 12 aa, Artificial Sequence
  Hinge
SPNMVPHAHHAQ

SEQ ID NO: 63 - DNA, 1515 bp, Artificial Sequence
  huFAP CAR
atggccttaccagtgaccgccttgctcctgccgctggccttgctgctccacgccgccaggccgggcagcgacatcgtgatgagccagagccccagcagcctggccgtgtccgtgggcgagaaagtgaccatgagctgcaagagcagccagagcctgctgtacagccggaaccagaagaactacctggcctggttccagcagaagcccggccagtcccccaagctgctgatcttctgggccagcacccgcgagagcggcgtgccagatagattcaccggcagcggcttcggcaccgacttcaacctgaccatcagcagcgtgcaggccgaggatctggccgtgtacgactgccagcagtacttcagctaccccctgaccttcggagccggcaccaagctggaactgaagggcggaggcggctctggcggcggaggaagttctggcggaggctctgaggtgcagctgcagcagagcggccctgagctggtgaaacctggcgccagcgtgaagatgtcctgcaagaccagccggtacaccttcaccgagtacaccatccactgggtgcgccagtctcacggcaagagcctggaatggatcggcggcatcaaccccaacaacggcatccccaactacaaccagaagttcaagggcagagccaccctgaccgtgggcaagtccagcagcaccgcctacatggaactgcggagcctgaccagcgaggacagcgctgtgtacttctgcgccagacggcggatcgcctacggctatgacgagggccacgccatggactactggggacagggcaccagcgtgaccgtgtctagctccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccatcgatagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 64 - DNA, 756 bp, Artificial Sequence
  huFAP-F19-L2H scFv
gacatcgtgatgagccagagccccagcagcctggccgtgtccgtgggcgagaaagtgaccatgagctgcaagagcagccagagcctgctgtacagccggaaccagaagaactacctggcctggttccagcagaagcccggccagtcccccaagctgctgatcttctgggccagcacccgcgagagcggcgtgccagatagattcaccggcagcggcttcggcaccgacttcaacctgaccatcagcagcgtgcaggccgaggatctggccgtgtacgactgccagcagtacttcagctaccccctgaccttcggagccggcaccaagctggaactgaagggcggaggcggctctggcggcggaggaagttctggcggaggctctgaggtgcagctgcagcagagcggccctgagctggtgaaacctggcgccagcgtgaagatgtcctgcaagaccagccggtacaccttcaccgagtacaccatccactgggtgcgccagtctcacggcaagagcctggaatggatcggcggcatcaaccccaacaacggcatccccaactacaaccagaagttcaagggcagagccaccctgaccgtgggcaagtccagcagcaccgcctacatggaactgcggagcctgaccagcgaggacagcgctgtgtacttctgcgccagacggcggatcgcctacggctatgacgagggccacgccatggactactggggacagggcaccagcgtgaccgtgtctagc